(19) United States
(12) Patent Application Publication
(10) Pub. No.: US 2025/0066786 A1
(43) Pub. Date: Feb. 27, 2025

(54) ANGELMAN SYNDROME ANTISENSE TREATMENT

(71) Applicant: The Texas A&M University System, College Station, TX (US)

(72) Inventor: Scott Victor Dindot, College Station, TX (US)

(21) Appl. No.: 18/944,351

(22) Filed: Nov. 12, 2024

Related U.S. Application Data
(63) Continuation of application No. 18/345,452, filed on Jun. 30, 2023, now Pat. No. 12,180,480.
Application No. 18/345,452 is a continuation of application No. 18/055,212, filed on Nov. 14, 2022, now Pat. No. 11,739,328.
Application No. 18/055,212 is a continuation of application No. 17/523,456, filed on Nov. 10, 2021, now Pat. No. 11,685,920.
Application No. 17/523,456 is a continuation of application No. 16/767,916, filed on May 28, 2020, now Pat. No. 11,198,869.
(60) Provisional application No. 62/593,431, filed on Dec. 1, 2017.
(60) Provisional application No. 62/676,034, filed on May 24, 2018.

(51) Int. Cl. C12N 15/113; C07H 21/04

(52) U.S. Cl. CPC C12N 15/113; C07H 21/04; C12N 2310/113; C12N 2310/341; C12N 2310/346; C12N 2320/30

(57) ABSTRACT

Disclosed herein are antisense oligonucleotides that are capable of inducing expression of ubiquitin-protein ligase E3A (UBE3A) from the paternal allele in animal or human neurons. The oligonucleotides target the suppressor of the UBE3A paternal allele by hybridization to SNHG14 long non-coding RNA at the 5′-end of UBE3A-AS, which is downstream of SNORD115-45 snoRNA. Also disclosed are pharmaceutical compositions and methods for treatment of Angelman syndrome.

### CROSS-REFERENCE TO RELATED APPLICATIONS

This application is a continuation of application Ser. No. 18/345,452, filed Jun. 30, 2023, which is a continuation of application Ser. No. 18/055,212, filed Nov. 14, 2022, which is a continuation of application Ser. No. 17/523,456, filed Nov. 10, 2021, which is a continuation of application Ser. No. 16/767,916, filed May 28, 2020, is a National Stage of International Application No. PCT/US2018/063416, filed Nov. 30, 2018, which claims benefit of U.S. Provisional Application No. 62/593,431, filed Dec. 1, 2017, and Application Ser. No. 62/676,034, filed May 24, 2018, which are hereby incorporated herein by reference in their entirety.

### SEQUENCE LISTING

This application contains a sequence listing filed in ST.26 format entitled “922001-1023 Sequence Listing” created on May 16, 2023 and having 956,036 bytes. The content of the sequence listing is incorporated herein in its entirety.

### BACKGROUND

Angelman syndrome (AS) is a neurodevelopmental disorder that is associated with severe cognitive and motor deficits, epilepsy, sleep-disorder, and an atypical ‘happy’ disposition. Individuals with AS are often diagnosed at 2-3 years of age and have a normal life-span. They require assisted living and medical care throughout their lives. There are currently few treatment options for individuals with AS, most of which involve anti-epileptic medications to treat seizures.

Angelman syndrome is caused by mutations that affect the expression or function of the maternally inherited ubiquitin-protein ligase E3A (UBE3A) gene. Unlike most genes, UBE3A is subject to genomic imprinting, which is a rare, naturally occurring phenomenon that turns-off one allele of a gene while leaving the other allele on. In neurons of the central nervous system (CNS), the paternal UBE3A allele is off, whereas in all other cell types of the body, both alleles of UBE3A are on. Because of this, AS is always caused by mutations that affect the maternally inherited UBE3A allele.

The paternal UBE3A allele is turned-off by the UBE3A antisense transcript (UBE3A-AS), which is a component of a long RNA transcript that expresses several protein coding and noncoding transcripts. UBE3A-AS is expressed from the paternal allele and only in neurons of the CNS and is both sufficient and necessary to turn-off expression of the paternal UBE3A allele. It's unclear why UBE3A is imprinted in neurons, but it creates a unique opportunity to treat individuals with AS, because there is a functional, albeit inactive, copy of UBE3A on the paternal chromosome. Studies to date indicate that turning on the paternal UBE3A allele is a viable therapy to treat AS.

### SUMMARY

Disclosed herein is a region in the 5′-end of UBE3A-AS transcript that is important for its stability. Based on these findings, antisense oligonucleotides (ASOs) were designed to target this region in order to terminate transcription of UBE3A-AS and reactivate expression of the paternal UBE3A allele. These ASOs targeting the 5′-end of UBE3A-AS are capable of stopping transcription of UBE3A-AS and turning on the paternal UBE3A allele. SNHG14 is a polycistronic transcript that encodes several different RNAs, including UBE3A-AS.

Accordingly, disclosed herein are ASOs containing a contiguous nucleotide sequence of 10 to 30 nucleotides (i.e., 10, 11, 12, 13, 14, 15, 16, 17, 18, 19, 20, 21, 22, 23, 24, 25, 26, 27, 28, 29, or 30) in length with at least 98% (i.e. 98%, 99%, or 100%) complementarity to target exons between the 3′-end of the SNORD115 and the 5′-end of SNORD109B, which is thought to represent the 5′-end of the UBE3A antisense transcript (UBE3A-AS). In particular the target exons can be in the 5′-end of UBE3A-AS, corresponding to position 25,511,577 to 25,516,681 on human chromosome 15 human genome assembly hg19. In some embodiments, the target nucleic acid is one of five exons located in the 5′-end of UBE3A-AS, which can correspond to positions 25,511,577 to 25,511,761 (exon 1), 25,512,059 to 25,512,191 (exon 2), 25,513,476 to 25,513,600 (exon 3), 25,514,752 to 25,514,880 (exon 4), and 25,516,565 to 25,516,681 (exon 5). Therefore, the target nucleic acid can be a contiguous nucleic acid sequence of 10 to 30 nucleotides within SEQ ID NO:1, 2, 3, 4, or 5.

In some embodiments, the target sequence is an exonic boundary involving UBE3A-AS exons 1-5, UBE3A-AS exon 5 and SNORD109B exon 1, and/or SNORD109B exons 1-2.

Methods and strategies for designing ASOs are known in the art. In some embodiments, the ASO is designed to target sequences that are conserved among human subjects. In some embodiments, the ASO is designed to target sequences that are conserved among primate subjects.

The oligonucleotide can be an antisense oligonucleotide (i.e., as will be understood by those of ordinary skill in the art—antisense to its target nucleic acid), e.g., with a gapmer design. The disclosed oligonucleotide is capable of inducing paternal UBE3A expression in a neuron by degradation, reduction, or removal of the UBE3A-AS transcript. It does this by targeting the 5′-end of UBE3A-AS at a site upstream of SNORD109B snoRNA. Examples of ASO designed to target exons 1-5 are provided in Tables 1, 2, 3, 4, or 5. For example, in some embodiments, the ASO comprises the nucleic acid sequence SEQ ID NO: 6, 7, 8, 9, 10, or 11.

The disclosed ASOs can also have one or more modifications to improve stability, solubility, activity, cellular distribution, and/or cellular uptake. For example, the disclosed ASO can contain one or more sugar-modified nucleosides and/or modified internucleoside linkages. For example, in some embodiments, the oligonucleotide comprises one or more internucleoside linkages modified from the natural phosphodiester to a linkage that is for example more resistant to nuclease attack. In some embodiments, the ASO contains one or more modified nucleobases that differ from naturally occurring nucleobases, but are functional during nucleic acid hybridization.

In some embodiments, the ASO is a DNA oligonucleotide. In some embodiments, the ASO is an RNA oligonucleotide. In still other embodiments, the ASO contains both deoxynucleotides and ribonucleotides. For example, the ASO can be a gapmer, headmer, or tailmer oligonucleotide. In some embodiments, the central block of a gapmer is flanked by blocks of modified ribonucleotides that protect the internal block from nuclease degradation. For example, the ASO can contain a stretch of 7, 8, 9, 10, or more natural DNA monomers to activate RNase H cleavage of the target RNA, along with 3, 4, or 5 modified ribonucleotide monomers at the 3′-and 5′-ends for protection against exonucleases. In some cases, the modified ribonucleotides are 2′-O-Methyl (OMe) RNA nucleotides, 2′-O-methoxyethyl (MOE)-modified nucleotides, or 2′-Locked Nucleic Acids (LNAs). Examples of gapmer ASOs are provided Tables 7, 11, and 17. Therefore, in some embodiments, the disclosed ASO has a nucleic acid sequence selected from SEQ ID NOs:362 to 392.

Also disclosed are pharmaceutical compositions comprising one or more of the ASOs disclosed herein and pharmaceutically acceptable diluents, carriers, salts and/or adjuvants.

Also disclosed are methods for in vivo or in vitro induction of UBE3A expression in a target cell where expression of paternal UBE3A is suppressed, by administering one or more of the disclosed ASOs or composition disclosed herein in an effective amount to said cell.

Also disclosed are methods for treating or preventing a disease, disorder or dysfunction associated with in vivo activity of UBE3A comprising administering a therapeutically or prophylactically effective amount of one or more of the disclosed ASOs to a subject suffering from or susceptible to the disease, disorder or dysfunction, such as Angelman syndrome.

The details of one or more embodiments of the invention are set forth in the accompanying drawings and the description below. Other features, objects, and advantages of the invention will be apparent from the description and drawings, and from the claims. For example, those skilled in the art, reading the specification will appreciate that the present disclosure demonstrates usefulness of certain sequences as described herein to impact expression of UBE3A, and furthermore teaches usefulness of oligonucleotide formats that are, or target (e.g., are complementary to), such sequences. Those skilled in the art will appreciate that the present disclosure is not limited to any particular mechanism of action—provided oligonucleotides may be useful regardless of whether they act via an antisense mechanism, for example, involving RNase H activity, and other therapeutic formats (e.g., siRNA, shRNA, nuclease gRNA, etc.) of oligonucleotides that are or target such sequences are also provided. Analogously, those skilled in the art will appreciate that the present disclosure, by defining useful sequences as described herein, also describes a variety of formats for such sequences (e.g., as part of a nucleic acid vector such as a vector from which they may be expressed (e.g., in vivo, in vitro, or both, etc.). Thus, those skilled in the art, reading the present disclosure, will appreciate that reference to “ASOs” herein is exemplary, and appropriate nucleic acids (e.g., oligonucleotides) may be utilized regardless of mechanism of action; those skilled in the art are aware of extensive literature regarding appropriate format and structure of nucleic acids (e.g., oligonucleotides) that operate via any of a variety of mechanisms (e.g., siRNA, shRNA, nuclease gRNA, etc.). In some embodiments, provided nucleic acids incorporate format and/or structural features known in the art to be useful in one or more mechanistic contexts (e.g., involving RNase H, RISC, a nucleic-acid-directed nuclease such as a Cas, etc.).

### DESCRIPTION OF DRAWINGS

FIGS. 1A to 1D illustrate the Prader-Willi/Angelman syndrome (PWS/AS) imprinted region in human and mouse. FIG. 1A shows RefSeq annotation of human PWS/AS imprinted region. FIG. 1B shows RefSeq annotation of PWS/AS imprinted orthologous region in mouse. FIG. 1C shows UBE3A-AS and 3′-end of UBE3A. FIG. 1D shows chain alignment showing orthologous regions between human, macaque (Cynomolgus macaque), pig, elephant, mouse, and rat. The target region is conserved among non-human primates but not rodents. FIG. 1D also shows genomic evolutionary rate profiling (GERP) plot of region. Positive values represent evolutionary constraint at specific DNA bases.

FIGS. 2A to 2E show an analysis of ASOs targeting mouse Ubeb3a-AS. FIG. 2A is a schematic of mouse Ube3a-AS transcript and approximate location of mouse-specific ASOs. Boxes and lines represent exons and introns, respectively. Arrow represents direction of transcription. FIG. 2B is a schematic of Ube3aYFP reporter allele used to measure paternal Ube3a protein levels. The Ube3aYFP mouse model was generated by targeting the yellow fluorescent protein (YFP) to the 3′-end of the endogenous Ube3a locus. Expression of Ube3a-AS inhibits transcription of the paternal Ube3aYFP allele, and loss of Ube3a-AS reactivates paternal Ube3aYFP expression, which can be detected by immunofluorescence imaging using an anti-YFP antibody. FIG. 2C is a schematic of experimental timeline to examine ASOs in mouse primary hippocampal neurons. Mouse primary hippocampal neurons were generated from newborn mice with a paternally inherited Ube3aYFP allele (0 DIV) and treated after 7 days in vitro (7 DIV). Three days post-treatment (10 DIV), Ube3aYFP protein levels were measured in individual cells. FIG. 2D contains immunofluorescent images showing paternal Ube3aYFP protein in primary neurons treated with vehicle (veh), a negative control ASO (ASO-C), Topotecan (Topo), ASO-B, and ASO 1.1. FIG. 2E shows mean paternal Ube3aYFP intensity levels in individual neuronal cells treated with vehicle (veh, 1% DMSO; n=3), control ASO (ASO-C, 15 μM; n=3), Topotecan (Topo, 0.3 μM; n=3), ASO-B (1, 5, 15 μM; n=3), ASO-1.1 (1, 5, 15 μM), ASO-1.2 (1, 5, 15 μM), and ASO 3.1 (1, 5, 15 μM). Abbreviations: YFP, yellow fluorescent protein; Tx, treatment; DIV, days in vitro; n.s., not significant. Error bars represent standard error of mean.

FIGS. 3A to 3D show analysis of ASOs targeting human UBE3A-AS. FIG. 3A is a schematic showing of human UBE3A-AS and approximate location of human-specific ASOs (ASOs 1-6). ASO-7 is located in an intron of UBE3A-AS. Boxes and lines represent exons and introns, respectively. FIG. 3B is a schematic of experimental timeline to examine ASOs in human GABAergic induced pluripotent stem cell (iPSC) derived neurons from a karyotypically normal individual. Human iPSC-derived neurons were treated after 14 DIV and then processed for RNA isolation at 20 DIV. FIGS. 3C and 3D show relative steady state RNA levels (normalized to ASO-C) of UBE3A-AS (FIG. 3C) and UBE3A (FIG. 3D) in iPSC-derived neurons treated with control ASO (ASO-C, 10 μM), and ASOs 1-7 (10 μM), and Topotecan (Topo, 1 μM). Abbreviations: Tx, treatment; DIV, days in vitro. Error bars represent standard error of mean.

FIGS. 4A to 4I show analysis of human ASO-4 and Topotecan in GABAergic iPSC-derived neurons. FIGS. 4A to 4F show relative expression (normalized to 1 nM) of UBE3A-AS (FIG. 4A), SNORD116 (FIG. 4B), IPW (FIG. 4C), SNORD115 (FIG. 4D), SNORD109A/B (FIG. 4E), and UBE3A (FIG. 4F) steady state RNA levels in iPSC-derived neurons treated with a 10-point ½ log dose curve of ASO-4 and Topotecan (1 nM, 3 nM, 10 nM, 30 nM, 100 nM, 300 nM, 1 μM, 3 μM, 10 μM, and 30 μM). FIG. 4G is a schematic of experimental timeline to examine ASO-4 in GABAergic iPSC-derived neurons treated at 59 DIV. FIG. 4H to 4I shows relative expression (normalized to ASO-C) of UBE3A-AS (FIG. 4H) and UBE3A (FIG. 4I) steady state RNA levels in iPSC-derived neurons treated with ASO-C (10 μM) and ASO-4 (1, 5, and 10 μM). Abbreviations: Tx, treatment. Error bars represent standard error of mean.

FIGS. 5A to 5F shows analysis of optimized ASOs in human GABAergic and glutamatergic iPSC-derived neurons. FIG. 5A is a schematic of experimental timeline to examine optimized ASOs in GABAergic iPSC-derived neurons. FIG. 5B shows relative expression of (normalized to water control) of UBE3A-AS steady state RNA levels in iPSC-derived neurons treated with a 5-point ½ log dose curve (30 nM, 100 nM, 300 nM, 1 μM, 3 μM; n=6) of ASO-3.1, ASO-3.2, ASO-4.1, ASO-4.2, ASO-4.3, ASO-4.4, ASO-6.1, ASO-4.I, and ASO-4.S. ASO-4.I and ASO-4.S represent ASO-4 manufactured by two companies (ASO-4.I, Integrated DNA Technologies; ASO-4.S, Sigma-Aldrich). FIG. 5C is a schematic of experimental timeline to examine ASO-4 and ASO-6.1 in GABAergic iPSC-derived neurons. FIG. 5D shows relative expression of (normalized to 1 nM) of UBE3A-AS and UBE3A steady state RNA levels in iPSC-derived neurons treated with a 10-point ½ log dose curve (1 nM, 3 nM, 10 nM, 30 nM, 100 nM, 300 nM, 1 μM, 3 μM, 10 μM, and 30 μM; n=3) of ASO-4 (ASO-4.I and ASO-4.S) and ASO-6.1. FIG. 5E is a schematic of experimental timeline to examine ASO-4 and ASO-6.1 in glutamatergic iPSC-derived neurons. FIG. 5F shows relative expression of (normalized to water control) of UBE3A-AS and UBE3A steady state RNA levels in iPSC-derived neurons treated with a 10-point ½ log dose curve (1 nM, 3 nM, 10 nM, 30 nM, 100 nM, 300 nM, 1 μM, 3 μM, 10 μM, and 30 uM; n=3) of ASO-4 (ASO-4.I and ASO-4.S) and ASO-6.1. Error bars represent standard error of mean.

FIG. 6A to 6D show identification of ASO target region in mouse PWS/AS imprinted region. FIG. 6A shows RefSeq annotation of the orthologous PWS/AS imprinted region on mouse chromosome 7C. FIG. 6B illustrates a transcript assembly generated from RNA-sequencing (RNA-seq) data from mouse brain. FIG. 6C shows ASO target region showing Snord115 snoRNAs retained in exons of the Snord115 host-gene transcript/5′-end of Ube3a-AS. Aligned RNA-seq reads are depicted below assembled transcripts. Exons and introns are depicted by boxes and lines, respectively. FIG. 6D is a sequence alignment of snoRNAs in retained exons
Snord115_ENSMUST00000101836 (SEQ ID NO:490),
Snord115_ENSMUST00000101936 (SEQ ID NO:491),
Snord115_ENSMUST00000104493 (SEQ ID NO:492),
Snord115_ENSMUST00000082443 (SEQ ID NO:493),
and Snord115_ENSMUST00000104427 (SEQ ID NO:494), showing retained snoRNAs have a degenerate C Box, which is required for functional snoRNA formation.

FIGS. 7A to 7G show identification of ASO target region in human PWS/AS imprinted region. FIG. 7A shows RefSeq annotation of Prader-Willi/Angelman syndrome (PWS/AS) imprinted region. FIG. 7B shows RNA-seq assembly of the human PWS polycistronic transcript. FIG. 7C shows SNORD115-45 is retained in an exon at the 3′-end of the SNORD115 host-gene transcript/5′-end of UBE3A-AS. Aligned RNA-seq reads generated from adult human brain showing L1 LINE is transcribed. FIG. 7D shows RefSeq annotation of 3′-end of SNORD115 cluster (SNORD115-39-48 and SNORD109B). FIG. 7E shows location of L1 LINE element between SNORD115-44 and SNORD115-45. FIG. 7F shows chain alignment of placental mammals representing major clades showing conservation at SNORD115-45-48 region, albeit reduced in rodents. FIG. 7G shows sequence alignment of snoRNAs in target region to SNORD115-44 (functional snoRNA) (SEQ ID NO:495), SNORD115-48 (SEQ ID NO:496), SNORD115-45 (SEQ ID NO:497), SNORD115-46 (SEQ ID NO:498), and SNORD115-47 (SEQ ID NO:499), showing SNORD115-45 (retained), SNORD115-46 (partially retained), and SNORD116-47 have degenerate C Box, which is required for functional snoRNA formation.

FIGS. 8A to 8C show pharmacodynamic analysis of candidate ASOs. FIG. 8A shows fitted dose response curves of normalized UBE3A-AS steady state RNA levels in GABAergic iPSC-derived neurons treated with a 10-point ½ log dose curve (1 nM, 3 nM, 10 nM, 30 nM, 100 nM, 300 nM, 1 μM, 3 μM, 10 μM, and 30 μM; n=2) of ASO-4 and ASO-6.1 with different backbone and RNA modification designs. Dose response curves fitted using a 4-parameter logistic regression model (Hill). Graphs represent fitted models and standard error. The Y axis represents relative UBE3A-AS RNA levels and X axis represents log molar (M) concentrations of ASO. FIGS. 8B and 8C are hierarchical clustering dendrogram and constellation plots of fitted dose response curves showing relationship between candidate ASOs and grouping into 3 clusters.

FIG. 9 shows pharmacodynamic analysis of ASO-6.1.PO-1.O and ASO-4.4.PS.L in Angelman syndrome iPSC-derived neurons. 4-Parameter logistic regression model (Hill) of normalized UBE3A-AS steady state RNA levels in Angelman syndrome iPSC-derived neurons treated with a 10-point ½ log dose curve (1 nM, 3 nM, 10 nM, 30 nM, 100 nM, 300 nM, 1 μM, 3 μM, 10 μM, and 30 μM; n=3) of ASO-6.1.PO-1.O and ASO-4.4.PS.L.

FIG. 10 shows expression analysis of RNAs encoded by the PWS polycistronic transcript in Angelman syndrome iPSC neurons treated with ASO-6.1-PO-1.O and ASO-4.4.PS.L. Shown are normalized steady state RNA levels of SNURF, SNRPN, SNHG116, SNORD116 snoRNAs, IPW, SNHG115, SNORD115 snoRNAs, UBE3A-AS, and UBE3A in AS iPSC-derived neurons treated with vehicle (1% H₂O; n=3), ASO-6.1.PO-1.O (30 μM; n=3), and ASO-4.4.PS.L (30 μM; n=3). Data represents mean percentage of RNA relative to vehicle. Error bars represent standard error of mean. Asterisk (*) denotes statistically significant differences (p<0.05) using one-way ANOVA with Dunnett's multiple comparison test relative to vehicle.

FIG. 11 shows pharmacodynamic analysis of ASO-6.1.PO-1.O and ASO-4.4.PS.L in Cynomolgus macaque. Shown are steady state RNA levels of UBE3A-AS in macaque CNS regions treated with vehicle (0.9% saline; n=5), ASO-6.1.PO-1.O (10 mg; n=3), and ASO-4.4.PS.L (10 mg; n=3). Data represents means percentage of UBE3A-AS RNA relative to vehicle. Error bars represent standard error of mean. Asterisk (*) denotes statistically significant differences (p<0.05) using one-way ANOVA with Dunnett's multiple comparison test relative to vehicle.

### DETAILED DESCRIPTION

The UBE3A-AS/Ube3a-AS transcript, otherwise known as ubiquitin-protein ligase E3A antisense transcript and UBE3A-AS/Ube3a-AS, is the name for the transcript generated by transcription of the UBE3A-AS transcript, which is on the antisense DNA strand relative to the UBE3A gene. Note that gene names with all caps indicate human genes (e.g. UBE3A) and gene names with only the first letter capped indicate mouse genes (e.g. Ube3a). The UBE3A-AS transcript is transcribed as part of a large polycistronic transcription unit that encodes SNURF-SNRPN, a cluster of orphan C/D box small nucleolar RNAs (SNORDs), and several uncharacterized long noncoding RNAs. In both mouse and human, the UBE3A/Ube3a gene is imprinted in neurons of the central nervous system, where it is expressed only from the maternal allele. The UBE3A-AS/Ube3a-AS transcript is both necessary and sufficient to silence transcription of the paternal UBE3A/Ube3a allele, and inhibition of UBE3A-AS/Ube3a-AS reactivates transcription of the paternal UBE3A/Ube3a allele. Mutations affecting the function or expression of the maternally inherited UBE3A allele cause Angelman syndrome (AS). In AS, the paternal allele is functional but epigenetically silenced. If unsilenced in AS patients, the paternal UBE3A allele could be a source of functional UBE3A in neurons.

The polycistronic transcription unit (hereafter referred to as the PTU) encoding UBE3A-AS is about 450,000 base-pairs long. Transcription of the PTU starts at upstream exons (U-exons) in the SNURF-SNRPN locus and stops towards the 5′-end of UBE3A. The PTU is organized (5′-3′) as follows: SNURF-SNRPN, SNORD107, SNORD64, SNORD109A, SNORD116 (29 copies), IPW, SNORD115 (48 copies), SNORD109B, and UBE3A, which is orientated in the opposite direction of the upstream transcripts. The polycistronic transcript is alternatively spliced and subject to alternative 3′-processing. SNURF-SNRPN encodes two polypeptides. The SNORDs are in the introns of a host-gene transcript (SNHG14) and are generated by exonucleolytic debranching of the spliced introns. UBE3A-AS represents the 3′-end of the transcript that overlaps the UBE3A gene. Most C/D box snoRNAs play a role in ribosome biogenesis where they direct 2′-O-methylation of ribosomal RNAs (rRNA); however, the snoRNAs located in the PWS/AS region lack any sequence complementarity to known rRNAs; however, the SNORD115 snoRNA has been found to change the alternative splicing of the serotonin receptor 2C pre-mRNA.

Disclosed herein is evidence that the 5′-end of UBE3A-AS transcript is important for its stability. As disclosed herein, ASOs targeting the 5′-end of UBE3A-AS are capable of reducing UBE3A-AS levels, presumably by stopping transcription of UBE3A-AS, and turning-on the paternal UBE3A allele.

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide disclosed herein is man-made, e.g., chemically synthesized. The oligonucleotide disclosed herein may also comprise one or more modified nucleosides or nucleotides.

The term “antisense oligonucleotide” as used herein is defined as oligonucleotides capable of modulating expression of a target gene by hybridizing to a target nucleic acid, in particular to a contiguous sequence on a target nucleic acid. In some embodiments, the antisense oligonucleotides disclosed herein are single stranded.

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide are present in the contiguous nucleotide sequence. In some embodiments the oligonucleotide comprises the contiguous nucleotide sequence and may, optionally comprise further nucleotide(s), for example a nucleotide linker region which may be used to attach a functional group to the contiguous nucleotide sequence. The nucleotide linker region may or may not be complementary to the target nucleic acid.

Nucleotides are the building blocks of oligonucleotides and polynucleotides, and can include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In some embodiments, the modified nucleoside comprises a modified sugar moiety. The term modified nucleoside may also be used herein interchangeably with the term “nucleoside analogue” or modified “units” or modified “monomers”.

The term “modified internucleoside linkage” is defined as generally understood by the skilled person as linkages other than phosphodiester (PO) linkages or natural phosphate linkages that covalently couples two nucleosides together. Nucleotides with modified internucleoside linkage are also termed “modified nucleotides”. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide disclosed herein, for example, within the gap region of a gapmer oligonucleotide, as well as in regions of modified nucleosides.

In some embodiments, the oligonucleotide comprises one or more internucleoside linkages modified from the natural phosphodiester to a linkage that is, for example, more resistant to nuclease attack. Nuclease resistance may be determined by incubating the oligonucleotide in blood serum or by using a nuclease resistance assay [e.g., snake venom phosphodiesterase (SVPD)], both are well known in the art. Internucleoside linkages which are capable of enhancing the nuclease resistance of an oligonucleotide are referred to as nuclease resistant internucleoside linkages.

In some embodiments at least 50% of the internucleoside linkages in the oligonucleotide, or contiguous nucleotide sequence thereof, are modified, such as at least 60%, such as at least 70%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified.

It will be recognized that, in some embodiments, the internucleoside linkages which link the oligonucleotide to a non-nucleotide functional group, such as a conjugate, may be phosphodiester. In some embodiments, the internucleoside linkages which link the oligonucleotide to a non-nucleotide functional group are modified.

In some embodiments all of the internucleoside linkages of the oligonucleotide, or contiguous nucleotide sequence thereof, are nuclease resistant internucleoside linkages.

Modified internucleoside linkages may, for example, be selected from the group comprising phosphorothioate, diphosphorothioate, and boranophosphate. In some embodiments, the modified internucleoside linkages are compatible with the RNase H recruitment of the oligonucleotide disclosed herein, for example, phosphorothioate, diphosphorothioate, or boranophosphate.

In some embodiments the internucleoside linkage comprises sulphur(S), such as a phosphorothioate internucleoside linkage.

A phosphorothioate internucleoside linkage is particularly useful due to nuclease resistance, beneficial pharmacokinetics and ease of manufacture. In preferred embodiments at least 50% of the internucleoside linkages in the oligonucleotide, or contiguous nucleotide sequence thereof, are phosphorothioate, such as at least 60%, such as at least 70%, such as at least 80%, or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

In some embodiments, the oligonucleotide comprises one or more neutral internucleoside linkage, particularly a internucleoside linkage selected from phosphotriester, methylphosphonate, MMI, amide-3, formacetal or thioformacetal. Further internucleoside linkages are disclosed in WO2009/124238 (incorporated herein by reference). In an embodiment the internucleoside linkage is selected from linkers disclosed in WO2007/031091 (incorporated herein by reference).

Nuclease resistant linkages, such as phosphorothioate linkages, are particularly useful in oligonucleotide regions capable of recruiting nuclease when forming a duplex with the target nucleic acid, such as region G for gapmers, or the non-modified nucleoside region of headmers and tailmers. Phosphorothioate linkages may, however, also be useful in non-nuclease recruiting regions and/or affinity enhancing regions such as regions F and F′ for gapmers, or the modified nucleoside region of headmers and tailmers.

Each of the design regions may however comprise internucleoside linkages other than phosphorothioate, such as phosphodiester linkages, in particularly in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide. WO2008/113832 is incorporated herein by reference for the teaching of oligonucleotides having phosphodiester linkages.

In some embodiments, all the internucleoside linkages in the oligonucleotide are phosphorothioate and/or boranophosphate linkages. In some embodiments, all the internucleoside linkages in the oligonucleotide are phosphorothioate linkages.

The term nucleobase includes the purine (e.g., adenine and guanine) and pyrimidine (e.g., uracil, thymine, and cytosine) moiety present in nucleosides and nucleotides which form hydrogen bonds in nucleic acid hybridization. The term nucleobase also encompasses modified nucleobases which may differ from naturally occurring nucleobases but are functional during nucleic acid hybridization. In this context “nucleobase” refers to both naturally occurring nucleobases, such as adenine, guanine, cytosine, thymidine, uracil, xanthine, and hypoxanthine, as well as non-naturally occurring variants.

In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl-cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include modified nucleobases of equivalent function. For example, in the exemplified oligonucleotides, the nucleobase moieties are selected from A, T, G, C, and 5-methyl cytosine (5 mC). Combinations of these modifications may also be used. For example, 5 mC LNA nucleosides may be used. Likewise, 2′-hydroxymethyl (2′-OMe) 5 mC may be used.

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example, 5-methyl cytosine is often used in place of cytosine, and as such the term complementarity encompasses Watson Crick base-paring between non-modified and modified nucleobases.

The term “% complementary” as used herein, refers to the number of nucleotides in percent of a contiguous nucleotide sequence in a nucleic acid molecule (e.g., oligonucleotide) which, at a given position, are complementary to (i.e., form Watson Crick base pairs with) a contiguous nucleotide sequence, at a given position of a separate nucleic acid molecule (e.g., the target nucleic acid). The percentage is calculated by counting the number of aligned bases that form pairs between the two sequences, dividing by the total number of nucleotides in the oligonucleotide and multiplying by 100. In such a comparison a nucleobase/nucleotide which does not align (form a base pair) is termed a mismatch.

The term “hybridizing” or “hybridizes” as used herein is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions, Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RTIn(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides disclosed herein hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

In some embodiments, the disclosed oligonucleotide comprises a contiguous nucleotide sequence of at least 8 nucleotides which is complementary to or hybridizes to a target sequence present in the target nucleic acid molecule. The contiguous nucleotide sequence (and therefore the target sequence) comprises of at least 8 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

In some embodiments, the disclosed oligonucleotide is a functional nucleic acid, such as a siRNA, shRNA, or nuclease gRNA, that inhibits, mutates, or deletes the target nucleic acid sequence.

The term “modulation of expression” as used herein is to be understood as an overall term for an oligonucleotide's ability to alter the amount of UBE3A RNA/protein when compared to the amount of UBE3A before administration of the oligonucleotide. Alternatively modulation of expression may be determined by reference to a control experiment where the disclosed oligonucleotide is not administered. The modulation effected by the oligonucleotide is related to its ability to reduce, remove, prevent, lessen, lower or terminate the suppression of the paternal UBE3A-AS transcript, i.e., by targeting the 5′-end of UBE3A-AS, which is downstream of SNORD115-45 snoRNA. The modulation can also be viewed as the oligonucleotide's ability to restore, increase or enhance expression of paternal UBE3A, e.g., by removal or blockage of inhibitory mechanisms affected by UBE3A-AS.

The disclosed oligonucleotide may comprise one or more nucleosides which have a modified sugar moiety, i.e., a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g., by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g., UNA). Other sugar modified nucleosides include, for example, bicyclohexose nucleosides (WO2011/017521) or tricyclic nucleosides (WO2013/154798). Modified nucleosides also include nucleosides where the sugar moiety is replaced with a non-sugar moiety, for example, in the case of peptide nucleic acids (PNA) or morpholino nucleic acids.

Sugar modifications also include modifications made via altering the substituent groups on the ribose ring to groups other than hydrogen, or the 2′-OH group naturally found in DNA and RNA nucleosides. Substituents may, for example, be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA (O-Me), 2′-alkoxy-RNA, 2′-O-methoxyethyl-RNA (MOE), 2′-amino-DNA, 2′-Fluoro-RNA, and 2′-fluoro-ANA (F-ANA). For further examples, please see Freier & Altmann; Nucl. Acid Res., 1997, 25, 4429-4443 and Uhlmann; Curr. Opinion in Drug Development, 2000, 3(2), 293-213; and Deleavey and Damha, Chemistry and Biology 2012, 19, 937.

Locked Nucleic Acid (LNA) nucleosides are modified nucleosides which comprise a linker group (referred to as a biradical or a bridge) between C2′ and C4′ of the ribose sugar ring of a nucleotide. These nucleosides are also termed bridged nucleic acid or bicyclic nucleic acid (BNA) in the literature.

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the disclosed oligonucleotides are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example, gapmers, headmers, and tailmers.

The term “gapmer” as used herein refers to an antisense oligonucleotide which comprises a region of RNase H recruiting oligonucleotides (gap) which is flanked 5′ and 3′ by one or more affinity enhancing modified nucleosides (flanks). Various gapmer designs are described herein. Headmers and tailmers are oligonucleotides capable of recruiting RNase H where one of the flanks is missing, i.e., only one of the ends of the oligonucleotide comprises affinity enhancing modified nucleosides. For headmers the 3′ flank is missing (i.e. the 5′ flank comprise affinity enhancing modified nucleosides) and for tailmers the 5′ flank is missing (i.e. the 3′ flank comprises affinity enhancing modified nucleosides).

Conjugation of the disclosed oligonucleotide to one or more non-nucleotide moieties may improve the pharmacology of the oligonucleotide, e.g., by affecting the activity, cellular distribution, cellular uptake, or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue, or cell type and thereby enhance the effectiveness of the oligonucleotide in that organ, tissue, or cell type. At the same time the conjugate may serve to reduce activity of the oligonucleotide in non-target cell types, tissues or organs, e.g., off target activity or activity in non-target cell types, tissues or organs. WO 93/07883 and WO 2013/033230 provides suitable conjugate moieties, which are hereby incorporated by reference. WO 2012/143379 provides a method of delivering a drug across the blood-brain-barrier by conjugation to an antibody fragment with affinity to the transferrin receptor, which are hereby incorporated by reference.

In some embodiments, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof. In some embodiments the non-nucleotide moiety an antibody or antibody fragment, such as an antibody or antibody fragment that facilitates delivery across the blood-brain-barrier, in particular an antibody or antibody fragment targeting the transferrin receptor.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms, which are within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter, which those skilled in the art will appreciate may be assessed at a particular point in time, such that in some embodiments, inhibition may be or comprise a delay in onset or reduction in frequency. In some embodiments, inhibition can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Antisense oligonucleotides (ASOs) were designed to target exons at the 5′-end of the SNORD115 host-gene transcript (AF400500), which encompasses SNORD115-46, SNORD115-47, SNORD115-48, and SNORD109B snoRNAs and is thought to represent the 5′-end of the UBE3A antisense transcript (UBE3A-AS). In particular the target nucleic acid can be the 5′-end of UBE3A-AS, corresponding to position 25,511,577 to 25,516,681 on human chromosome 15 human genome assembly hg19. In some embodiments, the target nucleic acid is one of five exons located in the 5′-end of UBE3A-AS, which can correspond to positions 25,511,577 to 25,511,761 (exon 1), 25,512,059 to 25,512,191 (exon 2), 25,513,476 to 25,513,600 (exon 3), 25,514,752 to 25,514,880 (exon 4), and 25,516,565 to 25,516,681 (exon 5).

Therefore, in some embodiments, the target nucleic acid is

(Exon 1, SEQ ID NO: 1)
ATGATGATATGGAAGAAAAGCACTCTTTGGCCTGTTGTGACTGGG
ACAGTTGACAGCACCCAGGTGTCCTTTAATGAAAATGCTCTTGAC
ACCAATGCATCCTAGCATCACAGCTTCAGGAAGCCTTCTCAAGTG
TGCATGGGGAGTACTATGTCTTTCATCAATAATGAAATCTTCTGA
TTTG.

In some embodiments, the target nucleic acid is

(Exon 2, SEQ ID NO: 2)
TAAGACATGCTGCCAAGAGATGTGCCATTCTATTATAAAAGATCA
GTAGCTTCCTTTACCGACGTGTATATTCTATCTAGAACATTGAGC
TATGGAAGACTCCCACCTAAGGGAATTAGTTTTACACCTTCAG.

In some embodiments, the target nucleic acid is

(Exon 3, SEQ ID NO: 3)
ATAAAGACTGCTGAGAAGAGCACCCTCTGGTGTTGTCACAGAGGC
AAGTGCTACCGCACAGGCATGCTGCAGTGAATTTAACTGATCCTC
TGTCCCTGCAACCGTTGTTTAAGGATGCTATTCTG.

In some embodiments, the target nucleic acid is

(Exon 4, SEQ ID NO: 4)
AAAAGACTGTGGAGGAAGAAAACCCTTTACCCTGTTGTTCAGGGA
GAAACTGACACCACTCAACTGCCTGGCACTGAAAATGTGGCATCC
AGTCCACTTTACCATCAGTGTTTAAGGAAACCATCTCTG.

In some embodiments, the target nucleic acid is

(Exon 5, SEQ ID NO: 5)
ATAAGGATGACTGAGGAAGAGTACTCTTTGGCTTGTTGACACCAG
CACAGCTGACACACCCAGATATCTGTTTGGTCTCCTGTGAACTTT
CAACCAGGATTTAAGGATGCCACTCTG.

In some embodiments, the disclosed ASO has the nucleic acid sequence

(ASO-1, SEQ ID NO: 6)
TAGAGGTGAAGGCCAGGCAC.

In some embodiments, the ASO has the nucleic acid sequence

(ASO-2, SEQ ID NO: 7)
GTACTCTTCCTCAGTCATCC.

In some embodiments, the disclosed ASO has the nucleic acid sequence

(ASO-3, SEQ ID NO: 8)
TGTCAGTTTCTCCCTGAACA.

In some embodiments, the disclosed ASO has the nucleic acid sequence

(ASO-4, SEQ ID NO: 9)
TAGAATGGCACATCTCTTGG.

In some embodiments, the disclosed ASO has the nucleic acid sequence

(ASO-6, SEQ ID NO: 10)
GTTTTCTTCCTCCACAGTCT.

In some embodiments, the disclosed ASO has the nucleic acid sequence

(ASO-7, SEQ ID NO: 11)
CTGGTGTCAACAAGCCAAAG.

Additional ASOs that can target exon 1 of the 3′-end of the SNORD115 region are provided below in Table 1. Example ASOs that can target exon 2 of the 3′-end of the SNORD115 are provided below in Table 2. Example ASOs that can target exon 3 of the 3′-end of the SNORD115 are provided below in Table 3. Example ASOs that can target exon 4 of the 3′-end of the SNORD115 are provided below in Table 4. Example ASOs that can target exon 5 of the 3′-end of the SNORD115 are provided below in Table 5.

TABLE 1
Exon 1 ASOs

| Target Sequence (5′→3′) | ASO (5′→3′) |
|---|---|
| GAAAAUGCUCUUGACACC (SEQ ID NO: 12) | GGTGTCAAGAGCATTTTC (SEQ ID NO: 15) |
| GAAAAUGCUCUUGACACCA (SEQ ID NO: 13) | TGGTGTCAAGAGCATTTTC (SEQ ID NO: 16) |
| GAAAAUGCUCUUGACACCAA (SEQ ID NO: 14) | TTGGTGTCAAGAGCATTTTC (SEQ ID NO: 17) |

TABLE 2
Exon 2 ASOs

| Target Sequence (5′→3′) | ASO (5′→3′) |
|---|---|
| CAUGCUGCCAAGAGAUGU (SEQ ID NO: 18) | ACATCTCTTGGCAGCATG (SEQ ID NO: 67) |
| CAUGCUGCCAAGAGAUGUG (SEQ ID NO: 19) | CACATCTCTTGGCAGCATG (SEQ ID NO: 68) |
| CAUGCUGCCAAGAGAUGUGC (SEQ ID NO: 20) | GCACATCTCTTGGCAGCATG (SEQ ID NO: 69) |
| AUGCUGCCAAGAGAUGUG (SEQ ID NO: 21) | CACATCTCTTGGCAGCAT (SEQ ID NO: 70) |
| AUGCUGCCAAGAGAUGUGC (SEQ ID NO: 22) | GCACATCTCTTGGCAGCAT (SEQ ID NO: 71) |
| AUGCUGCCAAGAGAUGUGCC (SEQ ID NO: 23) | GGCACATCTCTTGGCAGCAT (SEQ ID NO: 72) |
| UGCUGCCAAGAGAUGUGCC (SEQ ID NO: 24) | GGCACATCTCTTGGCAGCA (SEQ ID NO: 73) |
| UGCUGCCAAGAGAUGUGCCA (SEQ ID NO: 25) | TGGCACATCTCTTGGCAGCA (SEQ ID NO: 74) |
| GCUGCCAAGAGAUGUGCCA (SEQ ID NO: 26) | TGGCACATCTCTTGGCAGC (SEQ ID NO: 75) |
| GCUGCCAAGAGAUGUGCCAU (SEQ ID NO: 27) | ATGGCACATCTCTTGGCAGC (SEQ ID NO: 76) |
| CUGCCAAGAGAUGUGCCA (SEQ ID NO: 28) | TGGCACATCTCTTGGCAG (SEQ ID NO: 77) |
| CUGCCAAGAGAUGUGCCAU (SEQ ID NO: 29) | ATGGCACATCTCTTGGCAG (SEQ ID NO: 78) |
| CUGCCAAGAGAUGUGCCAUU (SEQ ID NO: 30) | AATGGCACATCTCTTGGCAG (SEQ ID NO: 79) |
| UGCCAAGAGAUGUGCCAU (SEQ ID NO: 31) | ATGGCACATCTCTTGGCA (SEQ ID NO: 80) |
| UGCCAAGAGAUGUGCCAUU (SEQ ID NO: 32) | AATGGCACATCTCTTGGCA (SEQ ID NO: 81) |
| UGCCAAGAGAUGUGCCAUUC (SEQ ID NO: 33) | GAATGGCACATCTCTTGGCA (SEQ ID NO: 82) |
| GCCAAGAGAUGUGCCAUU (SEQ ID NO: 34) | AATGGCACATCTCTTGGC (SEQ ID NO: 83) |
| GCCAAGAGAUGUGCCAUUC (SEQ ID NO: 35) | GAATGGCACATCTCTTGGC (SEQ ID NO: 84) |
| GCCAAGAGAUGUGCCAUUCU (SEQ ID NO: 36) | AGAATGGCACATCTCTTGGC (SEQ ID NO: 85) |
| CCAAGAGAUGUGCCAUUC (SEQ ID NO: 37) | GAATGGCACATCTCTTGG (SEQ ID NO: 86) |
| CCAAGAGAUGUGCCAUUCU (SEQ ID NO: 38) | AGAATGGCACATCTCTTGG (SEQ ID NO: 87) |
| CCAAGAGAUGUGCCAUUCUA (SEQ ID NO: 39) | TAGAATGGCACATCTCTTGG (SEQ ID NO: 88) |
| CAAGAGAUGUGCCAUUCU (SEQ ID NO: 40) | AGAATGGCACATCTCTTG (SEQ ID NO: 89) |
| CAAGAGAUGUGCCAUUCUA (SEQ ID NO: 41) | TAGAATGGCACATCTCTTG (SEQ ID NO: 90) |
| CAAGAGAUGUGCCAUUCUAU (SEQ ID NO: 42) | ATAGAATGGCACATCTCTTG (SEQ ID NO: 91) |
| UCCUUUACCGACGUGUAU (SEQ ID NO: 43) | ATACACGTCGGTAAAGGA (SEQ ID NO: 92) |
| UCCUUUACCGACGUGUAUA (SEQ ID NO: 44) | TATACACGTCGGTAAAGGA (SEQ ID NO: 93) |
| UCCUUUACCGACGUGUAUAU (SEQ ID NO: 45) | ATATACACGTCGGTAAAGGA (SEQ ID NO: 94) |
| CCUUUACCGACGUGUAUA (SEQ ID NO: 46) | TATACACGTCGGTAAAGG (SEQ ID NO: 95) |
| CCUUUACCGACGUGUAUAU (SEQ ID NO: 47) | ATATACACGTCGGTAAAGG (SEQ ID NO: 96) |
| CCUUUACCGACGUGUAUAUU (SEQ ID NO: 48) | AATATACACGTCGGTAAAGG (SEQ ID NO: 97) |
| ACCGACGUGUAUAUUCUAUC (SEQ ID NO: 49) | GATAGAATATACACGTCGGT (SEQ ID NO: 98) |
| CCGACGUGUAUAUUCUAUC (SEQ ID NO: 50) | GATAGAATATACACGTCGG (SEQ ID NO: 99) |
| CCGACGUGUAUAUUCUAUCU (SEQ ID NO: 51) | AGATAGAATATACACGTCGG (SEQ ID NO: 100) |
| UCUAGAACAUUGAGCUAUGG (SEQ ID NO: 52) | CCATAGCTCAATGTTCTAGA (SEQ ID NO: 101) |
| CAUUGAGCUAUGGAAGAC (SEQ ID NO: 53) | GTCTTCCATAGCTCAATG (SEQ ID NO: 102) |
| CUAUGGAAGACUCCCACCUA (SEQ ID NO: 54) | TAGGTGGGAGTCTTCCATAG (SEQ ID NO: 103) |
| UAUGGAAGACUCCCACCUA (SEQ ID NO: 55) | TAGGTGGGAGTCTTCCATA (SEQ ID NO: 104) |
| UAUGGAAGACUCCCACCUAA (SEQ ID NO: 56) | TTAGGTGGGAGTCTTCCATA (SEQ ID NO: 105) |
| AUGGAAGACUCCCACCUA (SEQ ID NO: 57) | TAGGTGGGAGTCTTCCAT (SEQ ID NO: 106) |
| AUGGAAGACUCCCACCUAA (SEQ ID NO: 58) | TTAGGTGGGAGTCTTCCAT (SEQ ID NO: 107) |
| UGGAAGACUCCCACCUAA (SEQ ID NO: 59) | TTAGGTGGGAGTCTTCCA (SEQ ID NO: 108) |
| GACUCCCACCUAAGGGAAUU (SEQ ID NO: 60) | AATTCCCTTAGGTGGGAGTC (SEQ ID NO: 109) |
| ACUCCCACCUAAGGGAAU (SEQ ID NO: 61) | ATTCCCTTAGGTGGGAGT (SEQ ID NO: 110) |
| ACUCCCACCUAAGGGAAUU (SEQ ID NO: 62) | AATTCCCTTAGGTGGGAGT (SEQ ID NO: 111) |
| ACUCCCACCUAAGGGAAUUA (SEQ ID NO: 63) | TAATTCCCTTAGGTGGGAGT (SEQ ID NO: 112) |
| CUCCCACCUAAGGGAAUU (SEQ ID NO: 64) | AATTCCCTTAGGTGGGAG (SEQ ID NO: 113) |
| CUCCCACCUAAGGGAAUUA (SEQ ID NO: 65) | TAATTCCCTTAGGTGGGAG (SEQ ID NO: 114) |
| UCCCACCUAAGGGAAUUA (SEQ ID NO: 66) | TAATTCCCTTAGGTGGGA (SEQ ID NO: 115) |

TABLE 3
Exon 3 ASOs

| Target Sequence (5′→3′) | ASO (5′→3′) |
|---|---|
| GAUAAAGACUGCUGAGAAGA (SEQ ID NO: 116) | TCTTCTCAGCAGTCTTTATC (SEQ ID NO: 139) |
| AUAAAGACUGCUGAGAAGAG (SEQ ID NO: 117) | CTCTTCTCAGCAGTCTTTAT (SEQ ID NO: 140) |
| UAAAGACUGCUGAGAAGAGC (SEQ ID NO: 118) | GCTCTTCTCAGCAGTCTTTA (SEQ ID NO: 141) |
| AAAGACUGCUGAGAAGAGCA (SEQ ID NO: 119) | TGCTCTTCTCAGCAGTCTTT (SEQ ID NO: 142) |
| AAGACUGCUGAGAAGAGCAC (SEQ ID NO: 120) | GTGCTCTTCTCAGCAGTCTT (SEQ ID NO: 143) |
| AGACUGCUGAGAAGAGCACC (SEQ ID NO: 121) | GGTGCTCTTCTCAGCAGTCT (SEQ ID NO: 144) |
| GACUGCUGAGAAGAGCACCC (SEQ ID NO: 122) | GGGTGCTCTTCTCAGCAGTC (SEQ ID NO: 145) |
| CAAGUGCUACCGCACAGGCA (SEQ ID NO: 123) | TGCCTGTGCGGTAGCACTTG (SEQ ID NO: 146) |
| AAGUGCUACCGCACAGGCAU (SEQ ID NO: 124) | ATGCCTGTGCGGTAGCACTT (SEQ ID NO: 147) |
| AGUGCUACCGCACAGGCAUG (SEQ ID NO: 125) | CATGCCTGTGCGGTAGCACT (SEQ ID NO: 148) |
| UGCUACCGCACAGGCAUGCU (SEQ ID NO: 126) | AGCATGCCTGTGCGGTAGCA (SEQ ID NO: 149) |
| UACCGCACAGGCAUGCUGCA (SEQ ID NO: 127) | TGCAGCATGCCTGTGCGGTA (SEQ ID NO: 150) |
| GCACAGGCAUGCUGCAGUGA (SEQ ID NO: 128) | TCACTGCAGCATGCCTGTGC (SEQ ID NO: 151) |
| CACAGGCAUGCUGCAGUGAA (SEQ ID NO: 129) | TTCACTGCAGCATGCCTGTG (SEQ ID NO: 152) |
| ACAGGCAUGCUGCAGUGAAU (SEQ ID NO: 130) | ATTCACTGCAGCATGCCTGT (SEQ ID NO: 153) |
| CAGGCAUGCUGCAGUGAAUU (SEQ ID NO: 131) | AATTCACTGCAGCATGCCTG (SEQ ID NO: 154) |
| AGGCAUGCUGCAGUGAAUUU (SEQ ID NO: 132) | AAATTCACTGCAGCATGCCT (SEQ ID NO: 155) |
| GGCAUGCUGCAGUGAAUUUA (SEQ ID NO: 133) | TAAATTCACTGCAGCATGCC (SEQ ID NO: 156) |
| GCAUGCUGCAGUGAAUUUAA (SEQ ID NO: 134) | TTAAATTCACTGCAGCATGC (SEQ ID NO: 157) |
| CAUGCUGCAGUGAAUUUAAC (SEQ ID NO: 135) | GTTAAATTCACTGCAGCATG (SEQ ID NO: 158) |
| GCAGUGAAUUUAACUGAUCC (SEQ ID NO: 136) | GGATCAGTTAAATTCACTGC (SEQ ID NO: 159) |
| UCCCUGCAACCGUUGUUUAA (SEQ ID NO: 137) | TTAAACAACGGTTGCAGGGA (SEQ ID NO: 160) |
| CCCUGCAACCGUUGUUUAAG (SEQ ID NO: 138) | CTTAAACAACGGTTGCAGGG (SEQ ID NO: 161) |

TABLE 4
Exon 4 ASOs

| Target Sequence (5′→3′) | ASO (5′→3′) |
|---|---|
| AAAAGACUGUGGAGGAAGA (SEQ ID NO: 162) | TCTTCCTCCACAGTCTTTT (SEQ ID NO: 237) |
| AAAAGACUGUGGAGGAAGAA (SEQ ID NO: 163) | TTCTTCCTCCACAGTCTTTT (SEQ ID NO: 238) |
| AAAGACUGUGGAGGAAGAA (SEQ ID NO: 164) | TTCTTCCTCCACAGTCTTT (SEQ ID NO: 239) |
| AAAGACUGUGGAGGAAGAAA (SEQ ID NO: 165) | TTTCTTCCTCCACAGTCTTT (SEQ ID NO: 240) |
| AAGACUGUGGAGGAAGAAAA (SEQ ID NO: 166) | TTTTCTTCCTCCACAGTCTT (SEQ ID NO: 241) |
| AGACUGUGGAGGAAGAAAAC (SEQ ID NO: 167) | GTTTTCTTCCTCCACAGTCT (SEQ ID NO: 242) |
| ACUGUGGAGGAAGAAAAC (SEQ ID NO: 168) | GTTTTCTTCCTCCACAGT (SEQ ID NO: 243) |
| ACUGUGGAGGAAGAAAACC (SEQ ID NO: 169) | GGTTTTCTTCCTCCACAGT (SEQ ID NO: 244) |
| ACUGUGGAGGAAGAAAACCC (SEQ ID NO: 170) | GGGTTTTCTTCCTCCACAGT (SEQ ID NO: 245) |
| CUGUGGAGGAAGAAAACC (SEQ ID NO: 171) | GGTTTTCTTCCTCCACAG (SEQ ID NO: 246) |
| CUGUGGAGGAAGAAAACCC (SEQ ID NO: 172) | GGGTTTTCTTCCTCCACAG (SEQ ID NO: 247) |
| AAAACCCUUUACCCUGUUG (SEQ ID NO: 173) | CAACAGGGTAAAGGGTTTT (SEQ ID NO: 248) |
| AAAACCCUUUACCCUGUUGU (SEQ ID NO: 174) | ACAACAGGGTAAAGGGTTTT (SEQ ID NO: 249) |
| AAACCCUUUACCCUGUUGUU (SEQ ID NO: 175) | AACAACAGGGTAAAGGGTTT (SEQ ID NO: 250) |
| UUGUUCAGGGAGAAACUG (SEQ ID NO: 176) | CAGTTTCTCCCTGAACAA (SEQ ID NO: 251) |
| UUGUUCAGGGAGAAACUGAC (SEQ ID NO: 177) | GTCAGTTTCTCCCTGAACAA (SEQ ID NO: 252) |
| UGUUCAGGGAGAAACUGA (SEQ ID NO: 178) | TCAGTTTCTCCCTGAACA (SEQ ID NO: 253) |
| UGUUCAGGGAGAAACUGAC (SEQ ID NO: 179) | GTCAGTTTCTCCCTGAACA (SEQ ID NO: 254) |
| UGUUCAGGGAGAAACUGACA (SEQ ID NO: 180) | TGTCAGTTTCTCCCTGAACA (SEQ ID NO: 255) |
| GUUCAGGGAGAAACUGACA (SEQ ID NO: 181) | TGTCAGTTTCTCCCTGAAC (SEQ ID NO: 256) |
| UCAGGGAGAAACUGACACCA (SEQ ID NO: 182) | TGGTGTCAGTTTCTCCCTGA (SEQ ID NO: 257) |
| CAGGGAGAAACUGACACCA (SEQ ID NO: 183) | TGGTGTCAGTTTCTCCCTG (SEQ ID NO: 258) |
| AGGGAGAAACUGACACCA (SEQ ID NO: 184) | TGGTGTCAGTTTCTCCCT (SEQ ID NO: 259) |
| AGGGAGAAACUGACACCAC (SEQ ID NO: 185) | GTGGTGTCAGTTTCTCCCT (SEQ ID NO: 260) |
| AGGGAGAAACUGACACCACU (SEQ ID NO: 186) | AGTGGTGTCAGTTTCTCCCT (SEQ ID NO: 261) |
| GGGAGAAACUGACACCAC (SEQ ID NO: 187) | GTGGTGTCAGTTTCTCCC (SEQ ID NO: 262) |
| GGGAGAAACUGACACCACU (SEQ ID NO: 188) | AGTGGTGTCAGTTTCTCCC (SEQ ID NO: 263) |
| GGGAGAAACUGACACCACUC (SEQ ID NO: 189) | GAGTGGTGTCAGTTTCTCCC (SEQ ID NO: 264) |
| GGAGAAACUGACACCACU (SEQ ID NO: 190) | AGTGGTGTCAGTTTCTCC (SEQ ID NO: 265) |
| GGAGAAACUGACACCACUC (SEQ ID NO: 191) | GAGTGGTGTCAGTTTCTCC (SEQ ID NO: 266) |
| GGAGAAACUGACACCACUCA (SEQ ID NO: 192) | TGAGTGGTGTCAGTTTCTCC (SEQ ID NO: 267) |
| GAGAAACUGACACCACUC (SEQ ID NO: 193) | GAGTGGTGTCAGTTTCTC (SEQ ID NO: 268) |
| GAGAAACUGACACCACUCA (SEQ ID NO: 194) | TGAGTGGTGTCAGTTTCTC (SEQ ID NO: 269) |
| GAGAAACUGACACCACUCAA (SEQ ID NO: 195) | TTGAGTGGTGTCAGTTTCTC (SEQ ID NO: 270) |
| AGAAACUGACACCACUCA (SEQ ID NO: 196) | TGAGTGGTGTCAGTTTCT (SEQ ID NO: 271) |
| AGAAACUGACACCACUCAA (SEQ ID NO: 197) | TTGAGTGGTGTCAGTTTCT (SEQ ID NO: 272) |
| AGAAACUGACACCACUCAAC (SEQ ID NO: 198) | GTTGAGTGGTGTCAGTTTCT (SEQ ID NO: 273) |
| GAAACUGACACCACUCAA (SEQ ID NO: 199) | TTGAGTGGTGTCAGTTTC (SEQ ID NO: 274) |
| GAAACUGACACCACUCAAC (SEQ ID NO: 200) | GTTGAGTGGTGTCAGTTTC (SEQ ID NO: 275) |
| GAAACUGACACCACUCAACU (SEQ ID NO: 201) | AGTTGAGTGGTGTCAGTTTC (SEQ ID NO: 276) |
| AAACUGACACCACUCAAC (SEQ ID NO: 202) | GTTGAGTGGTGTCAGTTT (SEQ ID NO: 277) |
| AAACUGACACCACUCAACU (SEQ ID NO: 203) | AGTTGAGTGGTGTCAGTTT (SEQ ID NO: 278) |
| AAACUGACACCACUCAACUG (SEQ ID NO: 204) | CAGTTGAGTGGTGTCAGTTT (SEQ ID NO: 279) |
| AACUGACACCACUCAACU (SEQ ID NO: 205) | AGTTGAGTGGTGTCAGTT (SEQ ID NO: 280) |
| AACUGACACCACUCAACUG (SEQ ID NO: 206) | CAGTTGAGTGGTGTCAGTT (SEQ ID NO: 281) |
| AACUGACACCACUCAACUGC (SEQ ID NO: 207) | GCAGTTGAGTGGTGTCAGTT (SEQ ID NO: 282) |
| ACUGACACCACUCAACUG (SEQ ID NO: 208) | CAGTTGAGTGGTGTCAGT (SEQ ID NO: 283) |
| ACUGACACCACUCAACUGC (SEQ ID NO: 209) | GCAGTTGAGTGGTGTCAGT (SEQ ID NO: 284) |
| ACUGACACCACUCAACUGCC (SEQ ID NO: 210) | GGCAGTTGAGTGGTGTCAGT (SEQ ID NO: 285) |
| CUGACACCACUCAACUGC (SEQ ID NO: 211) | GCAGTTGAGTGGTGTCAG (SEQ ID NO: 286) |
| CUGACACCACUCAACUGCC (SEQ ID NO: 212) | GGCAGTTGAGTGGTGTCAG (SEQ ID NO: 287) |
| CUGACACCACUCAACUGCCU (SEQ ID NO: 213) | AGGCAGTTGAGTGGTGTCAG (SEQ ID NO: 288) |
| UGACACCACUCAACUGCC (SEQ ID NO: 214) | GGCAGTTGAGTGGTGTCA (SEQ ID NO: 289) |
| UGACACCACUCAACUGCCU (SEQ ID NO: 215) | AGGCAGTTGAGTGGTGTCA (SEQ ID NO: 290) |
| UGACACCACUCAACUGCCUG (SEQ ID NO: 216) | CAGGCAGTTGAGTGGTGTCA (SEQ ID NO: 291) |
| GACACCACUCAACUGCCU (SEQ ID NO: 217) | AGGCAGTTGAGTGGTGTC (SEQ ID NO: 292) |
| GACACCACUCAACUGCCUG (SEQ ID NO: 218) | CAGGCAGTTGAGTGGTGTC (SEQ ID NO: 293) |
| GACACCACUCAACUGCCUGG (SEQ ID NO: 219) | CCAGGCAGTTGAGTGGTGTC (SEQ ID NO: 294) |
| ACACCACUCAACUGCCUG (SEQ ID NO: 220) | CAGGCAGTTGAGTGGTGT (SEQ ID NO: 295) |
| ACACCACUCAACUGCCUGG (SEQ ID NO: 221) | CCAGGCAGTTGAGTGGTGT (SEQ ID NO: 296) |
| ACACCACUCAACUGCCUGGC (SEQ ID NO: 222) | GCCAGGCAGTTGAGTGGTGT (SEQ ID NO: 297) |
| CACCACUCAACUGCCUGGCA (SEQ ID NO: 223) | TGCCAGGCAGTTGAGTGGTG (SEQ ID NO: 298) |
| GAAAAUGUGGCAUCCAGU (SEQ ID NO: 224) | ACTGGATGCCACATTTTC (SEQ ID NO: 299) |
| AAAAUGUGGCAUCCAGUC (SEQ ID NO: 225) | GACTGGATGCCACATTTT (SEQ ID NO: 300) |
| GCAUCCAGUCCACUUUACCA (SEQ ID NO: 226) | TGGTAAAGTGGACTGGATGC (SEQ ID NO: 301) |
| CAUCCAGUCCACUUUACC (SEQ ID NO: 227) | GGTAAAGTGGACTGGATG (SEQ ID NO: 302) |
| CAUCCAGUCCACUUUACCA (SEQ ID NO: 228) | TGGTAAAGTGGACTGGATG (SEQ ID NO: 303) |
| CAUCCAGUCCACUUUACCAU (SEQ ID NO: 229) | ATGGTAAAGTGGACTGGATG (SEQ ID NO: 304) |
| AUCCAGUCCACUUUACCA (SEQ ID NO: 230) | TGGTAAAGTGGACTGGAT (SEQ ID NO: 305) |
| AUCCAGUCCACUUUACCAU (SEQ ID NO: 231) | ATGGTAAAGTGGACTGGAT (SEQ ID NO: 306) |
| AUCCAGUCCACUUUACCAUC (SEQ ID NO: 232) | GATGGTAAAGTGGACTGGAT (SEQ ID NO: 307) |
| GUUUAAGGAAACCAUCUCUG (SEQ ID NO: 233) | CAGAGATGGTTTCCTTAAAC (SEQ ID NO: 308) |
| UUUAAGGAAACCAUCUCUGG (SEQ ID NO: 234) | CCAGAGATGGTTTCCTTAAA (SEQ ID NO: 309) |
| UUAAGGAAACCAUCUCUGG (SEQ ID NO: 235) | CCAGAGATGGTTTCCTTAA (SEQ ID NO: 310) |
| UAAGGAAACCAUCUCUGG (SEQ ID NO: 236) | CCAGAGATGGTTTCCTTA (SEQ ID NO: 311) |

TABLE 5
Exon 5 ASOs

| Target Sequence (5′→3′) | ASO (5′→3′) |
|---|---|
| AUAAGGAUGACUGAGGAAG (SEQ ID NO: 312) | CTTCCTCAGTCATCCTTAT (SEQ ID NO: 335) |
| AUAAGGAUGACUGAGGAAGA (SEQ ID NO: 313) | TCTTCCTCAGTCATCCTTAT (SEQ ID NO: 336) |
| UAAGGAUGACUGAGGAAG (SEQ ID NO: 314) | CTTCCTCAGTCATCCTTA (SEQ ID NO: 337) |
| UAAGGAUGACUGAGGAAGA (SEQ ID NO: 315) | TCTTCCTCAGTCATCCTTA (SEQ ID NO: 338) |
| UAAGGAUGACUGAGGAAGAG (SEQ ID NO: 316) | CTCTTCCTCAGTCATCCTTA (SEQ ID NO: 339) |
| AAGGAUGACUGAGGAAGA (SEQ ID NO: 317) | TCTTCCTCAGTCATCCTT (SEQ ID NO: 340) |
| AAGGAUGACUGAGGAAGAG (SEQ ID NO: 318) | CTCTTCCTCAGTCATCCTT (SEQ ID NO: 341) |
| AAGGAUGACUGAGGAAGAGU (SEQ ID NO: 319) | ACTCTTCCTCAGTCATCCTT (SEQ ID NO: 342) |
| AGGAUGACUGAGGAAGAG (SEQ ID NO: 320) | CTCTTCCTCAGTCATCCT (SEQ ID NO: 343) |
| AGGAUGACUGAGGAAGAGU (SEQ ID NO: 321) | ACTCTTCCTCAGTCATCCT (SEQ ID NO: 344) |
| AGGAUGACUGAGGAAGAGUA (SEQ ID NO: 322) | TACTCTTCCTCAGTCATCCT (SEQ ID NO: 345) |
| GGAUGACUGAGGAAGAGU (SEQ ID NO: 323) | ACTCTTCCTCAGTCATCC (SEQ ID NO: 346) |
| GGAUGACUGAGGAAGAGUA (SEQ ID NO: 324) | TACTCTTCCTCAGTCATCC (SEQ ID NO: 347) |
| GGAUGACUGAGGAAGAGUAC (SEQ ID NO: 325) | GTACTCTTCCTCAGTCATCC (SEQ ID NO: 348) |
| GAUGACUGAGGAAGAGUA (SEQ ID NO: 326) | TACTCTTCCTCAGTCATC (SEQ ID NO: 349) |
| GAUGACUGAGGAAGAGUAC (SEQ ID NO: 327) | GTACTCTTCCTCAGTCATC (SEQ ID NO: 350) |
| GAUGACUGAGGAAGAGUACU (SEQ ID NO: 328) | AGTACTCTTCCTCAGTCATC (SEQ ID NO: 351) |
| AUGACUGAGGAAGAGUAC (SEQ ID NO: 329) | GTACTCTTCCTCAGTCAT (SEQ ID NO: 352) |
| AUGACUGAGGAAGAGUACU (SEQ ID NO: 330) | AGTACTCTTCCTCAGTCAT (SEQ ID NO: 353) |
| AUGACUGAGGAAGAGUACUC (SEQ ID NO: 331) | GAGTACTCTTCCTCAGTCAT (SEQ ID NO: 354) |
| UGACUGAGGAAGAGUACU (SEQ ID NO: 332) | AGTACTCTTCCTCAGTCA (SEQ ID NO: 355) |
| UGACUGAGGAAGAGUACUC (SEQ ID NO: 333) | GAGTACTCTTCCTCAGTCA (SEQ ID NO: 356) |
| UGACUGAGGAAGAGUACUCU (SEQ ID NO: 334) | AGAGTACTCTTCCTCAGTCA (SEQ ID NO: 357) |

The disclosed oligonucleotide is capable of modulating expression of paternal UBE3A, in particular induction or up-regulation of paternally expressed UBE3A in neuronal cells. The modulation is achieved by hybridizing to the 5′-end of UBE3A-AS. In certain embodiments the oligonucleotide disclosed herein hybridizes to a sub-sequence of the target nucleic acid of SEQ ID NO:1 with a ΔG° below −10 kcal, such as with a ΔG° between −10 to −60 kcal, such as −12 to −40, such as from −15 to −30 kcal or −16 to −27 kcal such as −18 to −25 kcal.

In some embodiments the disclosed oligonucleotides are capable of increasing the expression of UBE3A by least 20% compared to the expression level of UBE3A in a neuronal cell treated with saline or a non-targeting oligonucleotide, more preferably by at least 30%, 35%, 40%, 45%, 50%, 55%, 60%, 80%, 100%, 120%, 150%, 160%, 170%, 180%, 190%, 200%, 210%, 220%, 230%, 240% or 250% compared to the expression level of UBE3A in a neuronal cell treated with saline or a non-targeting oligonucleotide. In some embodiments, the disclosed oligonucleotides are capable of decreasing the level of the SNHG14 transcript downstream of SNORD115-45 by at least 20% compared to the level of the SNHG14 transcript downstream of SNORD1115-45 in a neuronal cell treated with saline or a non-targeting oligonucleotide, more preferably by at least 30%, 40%, 50%, 60%, 70%, 80%, 90% or 95% compared to the level of the SNHG14 transcript downstream of SNORD115-45 in a neuronal cell treated with saline or a non-targeting oligonucleotide.

Target modulation by the disclosed oligonucleotide is triggered by hybridization between a contiguous nucleotide sequence of the oligonucleotide and the target nucleic acid. In some embodiments the disclosed oligonucleotide comprises mismatches between the oligonucleotide and the target nucleic acid. Despite mismatches hybridization to the target nucleic acid may still be sufficient to show a desired modulation of UBE3A expression. Reduced binding affinity resulting from mismatches may advantageously be compensated by increased number of nucleotides in the oligonucleotide and/or an increased number of modified nucleosides capable of increasing the binding affinity to the target, such as 2′ modified nucleosides, including LNA, present within the oligonucleotide sequence.

The disclosed antisense oligonucleotide can have a contiguous nucleotide sequence of 10 to 30 nucleotides in length with at least 90% complementary, such as at least 91%, such as at least 92%, such as at least 93%, such as at least 94%, such as at least 95%, such as at least 96%, such as at least 97%, such as at least 98%, or 100% complementarity to one of five exons located in the 5′-end of UBE3A-AS disclosed herein.

Oligonucleotide design refers to the pattern of nucleoside sugar modifications in the oligonucleotide sequence. The disclosed antisense oligonucleotide comprises sugar-modified nucleosides and may also comprise DNA, RNA, or arabino nucleic acid (ANA) nucleosides. In some embodiments, the oligonucleotide comprises sugar-modified nucleosides and DNA nucleosides. In some embodiments, the oligonucleotide comprises sugar-modified nucleosides and RNA nucleosides. In some embodiments, the oligonucleotide comprises sugar-modified nucleosides and ANA nucleosides.

In some embodiments, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides.

In some embodiments, the oligonucleotide comprises at least one modified internucleoside linkage. In some embodiments, the internucleoside linkages within the contiguous nucleotide sequence are phosphorothioate or boranophosphate internucleoside linkages.

In some embodiments, the disclosed antisense oligonucleotide comprises one or more sugar modified nucleosides, such as 2′ sugar modified nucleosides. Preferably the disclosed antisense oligonucleotides comprise one or more LNA nucleosides or 2′ sugar modified nucleoside wherein the 2′ position is replaced by a substituent independently selected from the group consisting of, —F; —CF₃, —CN, —N₃, —NO, —NO₂, —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), —N(C₂-C₁₀ alkynyl)₂, —O—(C₁-C₁₀ alkylene)-O—(C₁-C₁₀ alkyl), —O—(C₁-C₁₀ alkylene)-NH—(C₁-C₁₀ alkyl), —O—(C₁-C₁₀ alkylene)-NH(C₁-C₁₀ alkyl)₂, —NH—(C₁-C₁₀ alkylene)-O—(C₁-C₁₀ alkyl), and —N(C₁-C₁₀ alkyl)-(C₁-C₁₀ alkylene)-O—(C₁-C₁₀ alkyl).

In some embodiments, the disclosed oligonucleotides comprises at least one LNA unit, such as 1, 2, 3, 4, 5, 6, 7, or 8 LNA units, such as from 2 to 6 LNA units, such as from 3 to 7 LNA units, 4 to 8 LNA units or 3, 4, 5, 6 or 7 LNA units. In some embodiments, all the modified nucleosides are LNA nucleosides. In some embodiments, LNA comprises a 2′-4′ biradical bridge of -L-, wherein -L- is —O—CH₂—, wherein—CH₂—is optionally substituted. In some embodiments, LNA comprises a 2′-4′ biradical bridge of -L-, wherein -L- is —O—CH₂—. In some embodiments, LNA comprises a 2′-4′ biradical bridge of -L- , wherein -L- is —O—CH(Et)-. In a further embodiment, the oligonucleotide may comprise both beta-D-oxy-LNA, and one or more of the following LNA units: thio-LNA, amino-LNA, oxy-LNA, and/or ENA in either the beta-D or alpha-L configurations or combinations thereof. In a further embodiment, all LNA cytosine units are 5-methyl-cytosine. In some embodiments, the oligonucleotide or contiguous nucleotide sequence has at least 1 LNA unit at the 5′ end and at least 2 LNA units at the 3′ end of the nucleotide sequence.

In some embodiments, the disclosed oligonucleotide is capable of recruiting RNase H. In some embodiments, the oligonucleotide has a gapmer design or structure also referred herein merely as “Gapmer”. In a gapmer structure the oligonucleotide comprises at least three distinct structural regions a 5′-flank, a gap and a 3′-flank, F-G-F′ in ′5->3′ orientation. In this design, flanking regions F and F′ (also termed wing regions) comprise a contiguous stretch of modified nucleosides, which are complementary to the UBE3A-AS target nucleic acid, while the gap region, G, comprises a contiguous stretch of nucleotides which are capable of recruiting a nuclease, preferably an endonuclease such as RNase, for example, RNase H, when the oligonucleotide is in duplex with the target nucleic acid. Nucleosides which are capable of recruiting a nuclease, in particular RNase H, can be selected from the group consisting of DNA, alpha-L-oxy-LNA, 2′-Flouro-ANA and UNA. Regions F and F′, flanking the 5′ and 3′ ends of region G, preferably comprise non-nuclease recruiting nucleosides (nucleosides with a 3′ endo structure), more preferably one or more affinity enhancing modified nucleosides. In some embodiments, the 3′ flank comprises at least one LNA nucleoside, preferably at least 2 LNA nucleosides. In some embodiments, the 5′ flank comprises at least one LNA nucleoside. In some embodiments both the 5′ and 3′ flanking regions comprise a LNA nucleoside. In some embodiments all the nucleosides in the flanking regions are LNA nucleosides. In other embodiments, the flanking regions may comprise both LNA nucleosides and other nucleosides (mixed flanks), such as DNA nucleosides and/or non-LNA modified nucleosides, such as 2′ substituted nucleosides. In this case the gap is defined as a contiguous sequence of at least 5 RNase H recruiting nucleosides (nucleosides with a 2′ endo structure, preferably DNA) flanked at the 5′ and 3′ end by an affinity enhancing modified nucleoside, preferably LNA, such as beta-D-oxy-LNA. Consequently, the nucleosides of the 5′ flanking region and the 3′ flanking region which are adjacent to the gap region are modified nucleosides, preferably non-nuclease recruiting nucleosides. In oligonucleotides with mixed flanks where the flanks comprise DNA the 5′ and 3′ nucleosides are modified nucleosides.

Methods for manufacturing the disclosed oligonucleotides are known. In some cases, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand).

In some embodiments, oligonucleotide synthesis methodologies are utilized that provide control of stereochemistry at one or more modified internucleoside linkages that include(s) a chiral atom. See, for example, WO2010/064146, WO2014/012081, WO2015/107425, WO2016/079183, WO2016/079181, WO2016/096938, WO2017/194498, and WO2018/177825, which are incorporated by reference for these methodologies.

Those skilled in the art will appreciate that useful nucleic acids provided by the present disclosure include those that store and/or express sequences of oligonucleotides described herein. In some embodiments, such nucleic acids may be or comprise vectors appropriate for delivery into and/or replication and/or expression in a cell (e.g., a microbial cell, for example for production and/or a mammalian cell, for example for treatment). Those skilled in the art are aware of a variety of technologies (e.g., recombinant nucleic acid technologies such as, for instance, that utilize one or more of amplification such as by polymerase chain reaction, cleavage such as by restriction digestion, linkage such as by ligation—whether in vitro or in vivo e.g., by gap repair, etc.).

Also disclosed are pharmaceutical compositions comprising any of the aforementioned oligonucleotides and/or oligonucleotide conjugates and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments, the diluent is artificial cerebrospinal fluid (aCSF).

The disclosed oligonucleotides may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Those skilled in the art are aware of a variety of formulation strategies useful for storage and/or administration of nucleic acid therapeutics such as oligonucleotide therapeutics. See, for example, Pushpendra et al “Nucleic Acids as Therapeutics” in From Nucleic Acid Sequences to Molecular Medicines, ed. Erdmann and Barciszewski, Springer-Verlag, 2012; Juliano “The Delivery of Therapeutic Oligonucleotides” Nuc. Acids. Res. 44:6518, 2016; etc.

In some embodiments, the oligonucleotide is formulated as a prodrug. In particular with respect to oligonucleotide conjugates, the conjugate moiety can be cleaved off the oligonucleotide once the prodrug is delivered to the site of action, e.g., the target cell.

Also disclosed are methods for treating or preventing a disease, comprising administering a therapeutically or prophylactically effective amount of an oligonucleotide, an oligonucleotide conjugate or a pharmaceutical composition disclosed herein to a subject suffering from or susceptible to the disease.

Also disclosed is use of the disclosed oligonucleotides for the manufacture of a medicament for the treatment of a disorder as referred to herein, or for a method of the treatment of as a disorder as referred to herein.

The disclosed pharmaceutical compositions may be administered by topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal) administration. In some embodiments, the disclosed pharmaceutical compositions are administered by a parenteral route including intravenous, intraarterial, subcutaneous, intraperitoneal or intramuscular injection or infusion, intrathecal or intracranial, e.g., intracerebral or intraventricular, administration. In some embodiments, the oligonucleotide is administered by intracerebral or intracerebroventricular injection. In another embodiment the active oligonucleotide or oligonucleotide conjugate is administered intrathecally. In some embodiments, the pharmaceutical composition is administered by intracisternae magna injection.

In some embodiments, AS therapy with pharmaceutical compositions described herein is administered to subject(s) suffering from or susceptible to AS. In some embodiments, a subject has been determined to have genetic characteristic associated with a defect in a maternal UBE3A gene. In some embodiments, an AS-associated genetic characteristic is or comprises a maternal deletion. In some embodiments, an AS-associated genetic characteristic is or comprises uniparental disomy. In some embodiments, an AS-associated genetic characteristic is or comprises a UBE3A mutation. In some embodiments, an AS-associated genetic characteristic is or comprises an imprinting defect.

In some embodiments, a subject has been determined to have one or more developmental history and/or laboratory finding characteristics that have been associated with AS such as, for example, one or more of:
(i) normal prenatal and birth history with normal head circumference and absence of major birth defects;
(ii) feeding difficulties as a neonate and/or as an infant;
(iii) developmental delay evident by 6-12 months of age, sometimes associated with truncal hypotonus;
(iv) unsteady limb movements and/or increased smiling;
(v) delayed but forward progression of development (no loss of skills);
(vi) normal metabolic, hematologic and chemical laboratory profiles;
(vii) structurally normal brain when assessed using MRI or CT (may have mild cortical atrophy or dysmyelination).

Alternatively or additionally, in some embodiments, a subject has been determined to display one or more clinical features that are consistently associated with AS such as, for example, one or more of:
(i) developmental delay, functionally severe
(ii) movement or balance disorder, usually ataxia of gait and/or tremulous movement of limbs. In some embodiments, such movement disorder can be mild. In some embodiments, such movement disorder may not appear as frank ataxia but can be or involve, for example, forward lurching, unsteadiness, clumsiness, or quick, jerky motion;
(iii) behavioral uniqueness: any combination of frequent laughter/smiling; apparent happy demeanor; easily excitable personality, often with uplifted hand-flapping or waving movements; hypermotoric behavior
(iv) speech impairment, such as for example absent or minimal use of words; alternatively or additionally, receptive and non-verbal communication skills higher than verbal ones.

Alternatively or additionally, in some embodiments, a subject has been determined to display one or more clinical features that are frequently (e.g., about 80% of the time) associated with AS such as, for example, one or more of:
(i) delayed, disproportionate growth in head circumference, usually resulting in microcephaly (≤2 S.D. of normal OFC) by age 2 years. In some embodiments, microcephaly is more pronounced in those with 15q11.2-q13 deletions;
(ii) seizures, onset usually <3 yrs. of age. In some embodiments, seizure severity may decrease with age but regardless, in some embodiments, the seizure disorder lasts throughout adulthood.
(iv) abnormal EEG, with a characteristic pattern, as is known in the art. In some embodiments, EEG abnormalities can occur in the first 2 years of life and can precede clinical features, and may not be correlated to clinical seizure events.

Alternatively or additionally, in some embodiments, a subject has been determined to display one or more clinical features that are sometimes (e.g., about 20-80% of the time) associated with AS such as, for example, one or more of:
(i) flat occiput
(ii) occipital groove
(iii) protruding tongue
(iv) tongue thrusting; suck/swallowing disorders
(v) feeding problems and/or truncal hypotonia during infancy
(vi) prognathia
(vii) wide mouth, wide-spaced teeth
(viii) frequent drooling
(ix) excessive chewing/mouthing behaviors
(x) strabismus
(xi) hypopigmented skin, light hair and eye color, in some embodiments determined as compared to family, and typically seen only in deletion cases
(xii) hyperactive lower extremity deep tendon reflexes
(xiii) uplifted, flexed arm position especially during ambulation
(xiv) wide-based gait with pronated or valgus-positioned ankles
(xv) increased sensitivity to heat
(xvi) abnormal sleep wake cycles and diminished need for sleep
(xvii) attraction to/fascination with water; fascination with crinkly items such as certain papers and plastics
(xviii) abnormal food related behaviors
(xix) obesity (in the older child)
(xx) scoliosis
(xxi) constipation

In some embodiments, a therapeutic regimen for the treatment of AS with a nucleic acid therapeutic (e.g., an oligonucleotide therapeutic such as an ASO) as described herein is or comprises administration of one or more doses of a pharmaceutical composition that comprises and/or delivers an oligonucleotide as described herein.

In some embodiments, a subject to whom a provided therapeutic regimen is administered is receiving or has received one or more other AS therapeutics including, for example, one or more other nucleic acid therapeutics (e.g., one or more other oligonucleotides that target UBE3A-AS). See, for example, WO2014004572A3, U.S. Pat. No. 9,617,539B2, US20170362592A1, and EP2864479B1.

In some embodiments, a subject to whom a provided therapeutic regimen is administered has suffered or is suffering from one or more seizures and/or is receiving or has received anti-seizure therapy. For example. In some embodiments, a subject may have received or be receiving one or more of valproic acid, clonazepam, phenobarbital, topiramate, carbamazepine, lamotrigine, leveltiracetam, phenytoin, zonisamide, ethosuxaminde, gabapentin, felbatame, oxcarbazepine, tranxene, ACTS, nitrazapam, pregabalin, mysoline, vigabatrin, etc. In some particular embodiments, a subject may have received or be receiving one or more of valproic acid, clonazepam, phenobarbital, topiramate, carbamazepine, lamotrigine, and/or levetiracetam.

Alternatively or additionally, in some embodiments, a subject may have received or be receiving dietary therapy such as, for example, a ketogenic diet, low glycemic index therapy, etc.

Still further alternatively or additionally, in some embodiments, a subject may have received or be receiving treatment with a vagal nerve stimulator.

As will be apparent to those skilled in the art reading the present disclosure, provided methods of treatment involve administering one or both of an oligonucleotide as described herein and an additional therapy (e.g., an alternative oligonucleotide and/or anti-epileptic therapy and/or one or more other therapeutic interventions), so that the subject receives combination therapy (e.g., is simultaneously exposed thereto, for example via overlapping dosing etc.). Also disclosed is the use of an oligonucleotide disclosed herein for the manufacture of a medicament wherein the medicament is in a dosage form for intrathecal administration.

Also disclosed is the use of an oligonucleotide disclosed herein for the manufacture of a medicament wherein the medicament is in a dosage form for intracerebral or intraventricular administration.

Also disclosed is the use of an oligonucleotide disclosed herein for the manufacture of a medicament wherein the medicament is in a dosage form for intracerebroventricular administration.

In some embodiments the oligonucleotide disclosed herein is for use in a combination treatment with another therapeutic agent. The therapeutic agent can for example be anticonvulsant medication.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Results

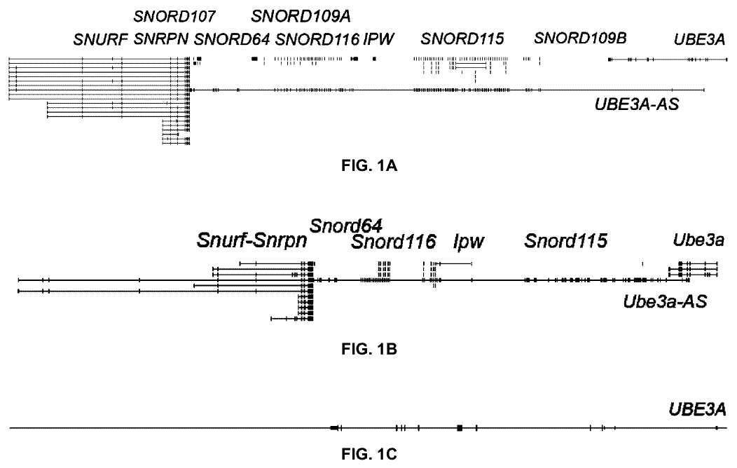

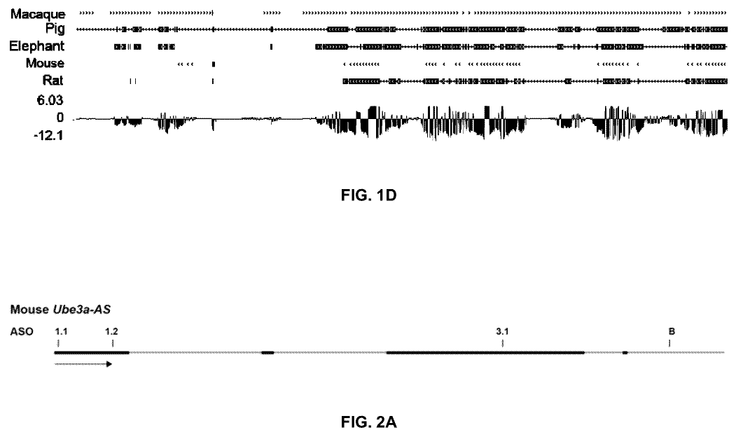

RNA-sequencing analysis of mouse and human CNS identified a region believed to be important for the stability and/or transcription of UBE3A-AS. Further analysis of the region showed low levels of sequence conservation between mouse and human (FIGS. 1A-1D).

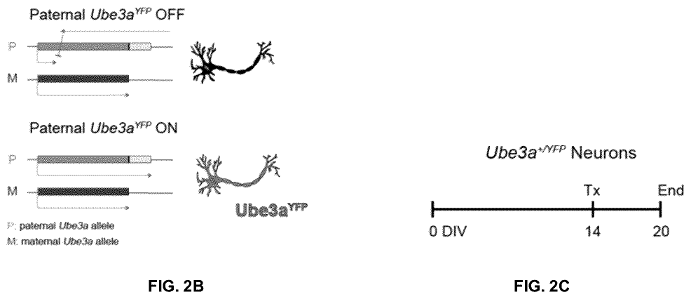

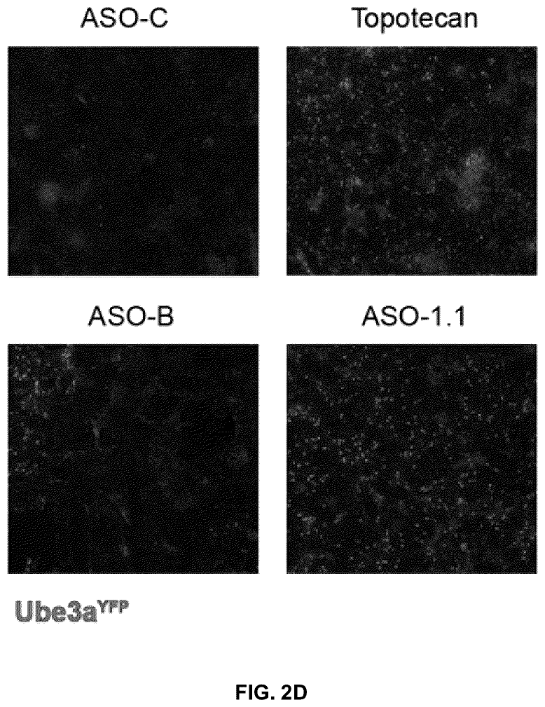

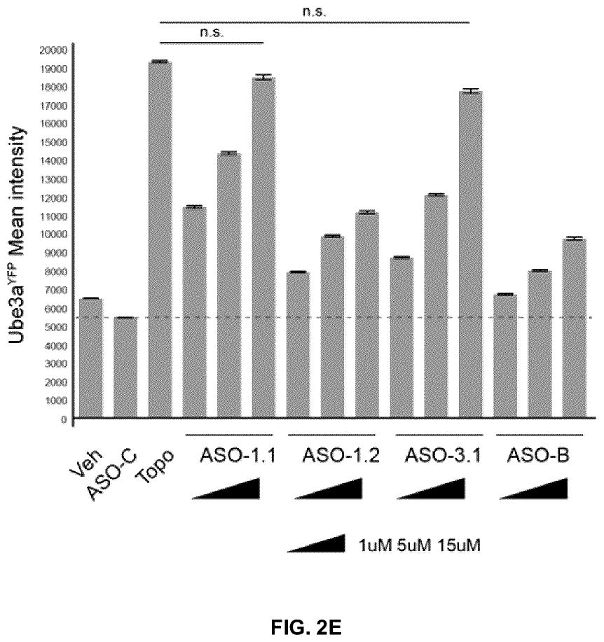

Based on these findings, mouse-specific ASOs were designed to target a specific region in the Ube3a-AS transcript (Table 6 and FIG. 2A). To test whether ASOs targeting this region reactivate expression of the paternal Ube3a allele, primary hippocampal neuronal cultures were generated from the Ube3aYFP reporter mouse model (Ube3a+/YFP; FIG. 2B) and treated at 7 days in vitro (DIV) with a control ASO [ASO-C (10 uM, n=3)], three ASOs targeting Ube3a-AS [ASO-1.1, ASO-1.2, ASO-3.1 (1 μM, 5 μM, and 15 μM, n=3)], and ASO-B (1 μM, 5 μM, and 15 μM, n=3)]. As a positive control, neurons were also treated with Topotecan [Topo (300 nM, n=3)] and a negative vehicle control [Veh (1%, n=3); FIG. 2 C]. Three days post-treatment (10 DIV), immunofluorescent imaging was used to quantify paternal Ube3aYFP protein levels in individual cells. Compared to controls (ASO-C and Veh), each treatment substantially increased paternal Ube3aYFP protein levels, with similar levels achieved in ASO-1.1 (15 μM), ASO-3.1 (15 μM), and Topotecan treatments (FIGS. 2D and 2E).

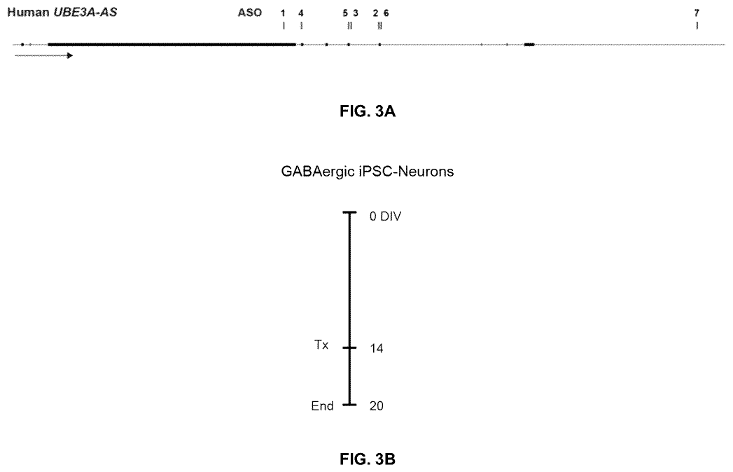

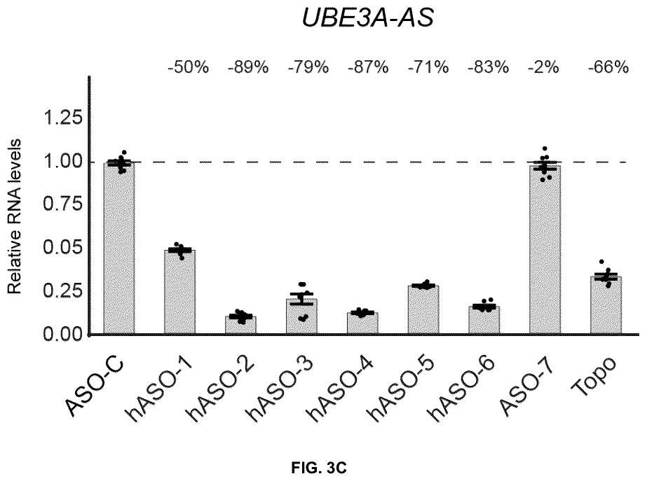

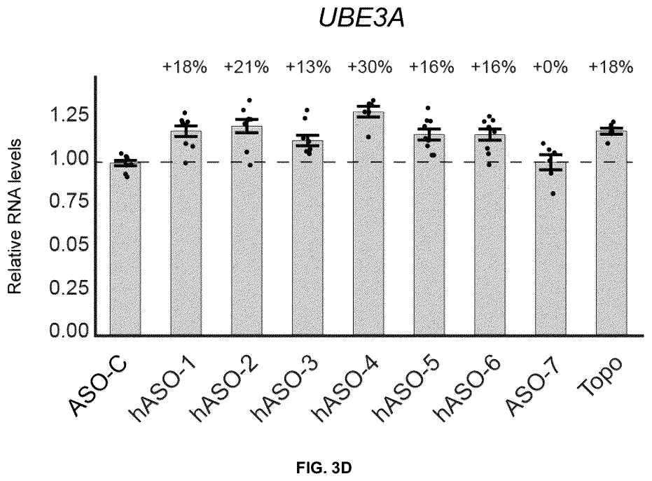

Human-specific ASOs were then designed to target this region, which included four ASOs targeting non-polymorphic regions in human and regions conserved (100%) with macaque (Rhesus and Cynomolgus) (Table 7 and FIG. 3A). Human induced pluripotent stem cell (iPSC) neural precursor cells were differentiated into GABAergic neurons for 14 DIV and then treated with a control ASO [ASO-C (10 μM, n=3)], Topotecan [Topo (1 μM, n=2)], and six ASOs targeting UBE3A-AS [ASO-1, ASO-2, ASO-3, ASO-4, ASO-5, and ASO-6 (10 μM, n=3)]. Additionally, an ASO targeting an intronic region downstream of SNORD109B was included (ASO-7). Six days post-treatment (20 DIV), RNA was isolated from the neurons and the steady state RNA levels of UBE3A-AS and UBE3A were estimated relative to the control treatment (FIG. 3B). With the exception of ASO-7, each ASO significantly decreased UBE3A-AS RNA levels, with ASO-2 and ASO-4 having the largest effect (Table 8 and FIG. 3C). UBE3A RNA levels also increased after treatment with each ASO (FIG. 3D).

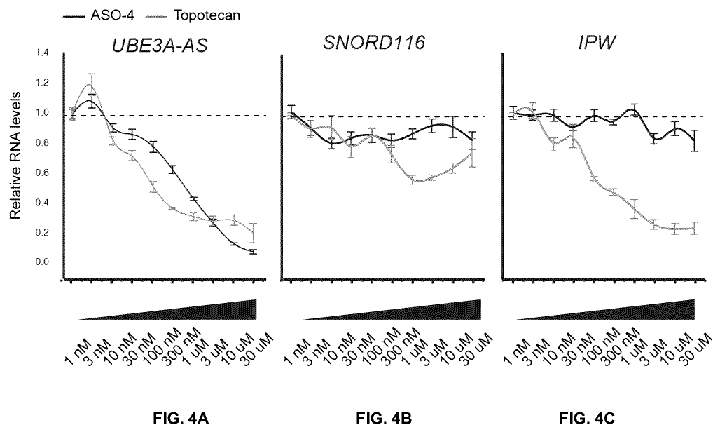

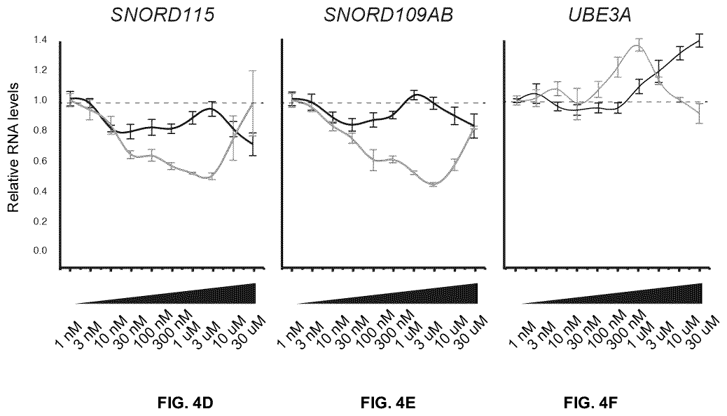

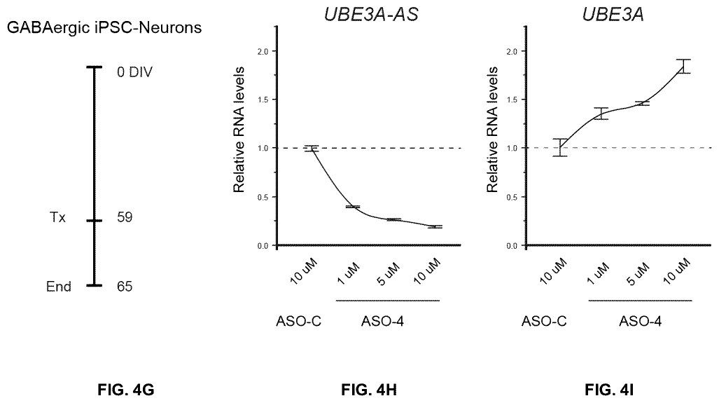

The potency of ASO-4 was further examined given its effect on UBE3A-AS RNA levels. GABAergic iPSC-derived neurons were treated at 14 DIV with a 10-point ½ log dose response curve of ASO-4 and Topotecan, as a positive control and for comparisons between treatment [1 nM, 3 nM, 10 nM, 30 nM, 100 nM, 300 nM, 1 μM, 3 μM, 10 μM, and 30 μM (ASO-4, n=6; Topotecan, n=2)]. At 20 DIV, the steady state RNA levels of UBE3A-AS were measured and dose response curves fitted to estimate the IC₅₀ and Eₘₐₓ (i.e., maximum UBE3A-AS inhibition) (Table 9 and FIG. 4A). The dose response curves of ASO-4 and Topotecan were significantly different (Parallelism test: F_{(3,145)}=11.2, p<0.0001), thus the relative potencies were not estimated. An equivalence test indicated that the IC₅₀ and Eₘₐₓ of ASO-4 and Topotecan were not equivalent [ASO-4/Topotecan IC₅₀ ratio:=1.2 (Lower confidence limit=1.1; Upper confidence limit=1.3); Emax ratio=−4.1 (Lower confidence limit=−12.9; Upper confidence limit=4.8)].

The effects of ASO-4 and Topotecan were then examined on the SNORD116, IPW, SNORD115, and SNORD109A RNAs, which are located upstream of the ASO-4 target region (see FIG. 1A). With the exception of SNORD116, ASO-4 had a significant effect on the RNA levels of IPW, SNORD115, and SNORD109A/B but not in a dose dependent manner. In contrast, Topotecan had a significant effect on SNORD116, IPW, SNORD115, and SNORD109A/B RNA levels that was dose dependent (Table 10 and FIGS. 4B-4E). Both ASO-4 and Topotecan increased total UBE3A RNA levels in a dose-dependent manner, except for Topotecan at higher concentrations (3 μM, 10 μM, and 30 μM; FIG. 4F).

The potency of ASO-4 was further examined in iPSC-derived neurons at a later time point in differentiation. GABAergic iPSC-derived neurons were treated at 59 DIV with a control ASO [ASO-C, 10 μM (n=3)] and ASO-4 [1 uM, 5 μM, and 10 μM (n=3)], and the steady state RNA levels of UBE3A-AS and UBE3A were measured as described above (FIG. 4G). Unlike neurons treated with ASO-4 at an earlier time point, the RNA levels of UBE3A and UBE3A-AS were highly inversely correlated (FIGS. 4H and 4I). For example, the effect of ASO-4 (10 μM) on UBE3A-AS RNA levels was similar between neurons treated at 14 and 59 DIV [20 DIV: UBE3A-AS: ↓87% (95% confidence intervals (CI): 80 to 95%); 65 DIV: ↓81% (95% CI: 74 to 88%)], whereas the effect of ASO-4 on UBE3A RNA levels was substantially larger in neurons treated at 59 DIV [20 DIV: ↑30% (95% CI: 16 to 44%); 65 DIV: 52 86% (95% CI: 59% to 113%)].

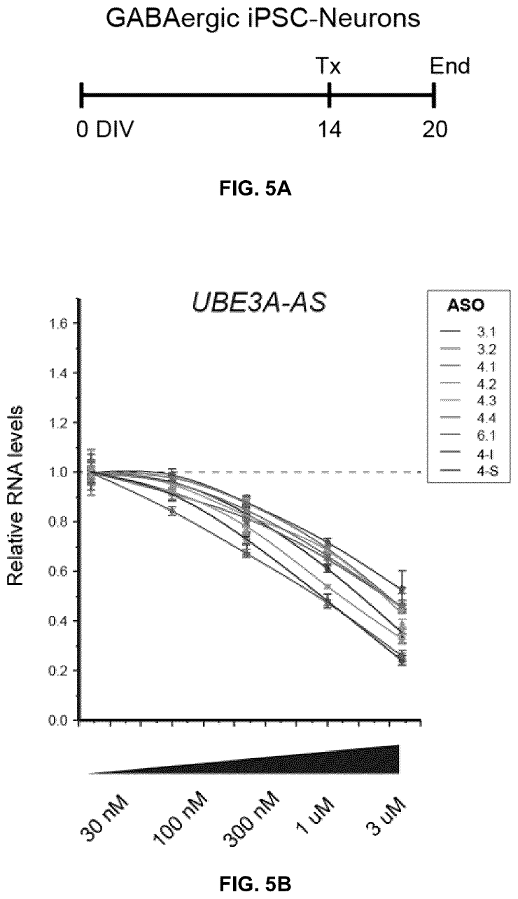

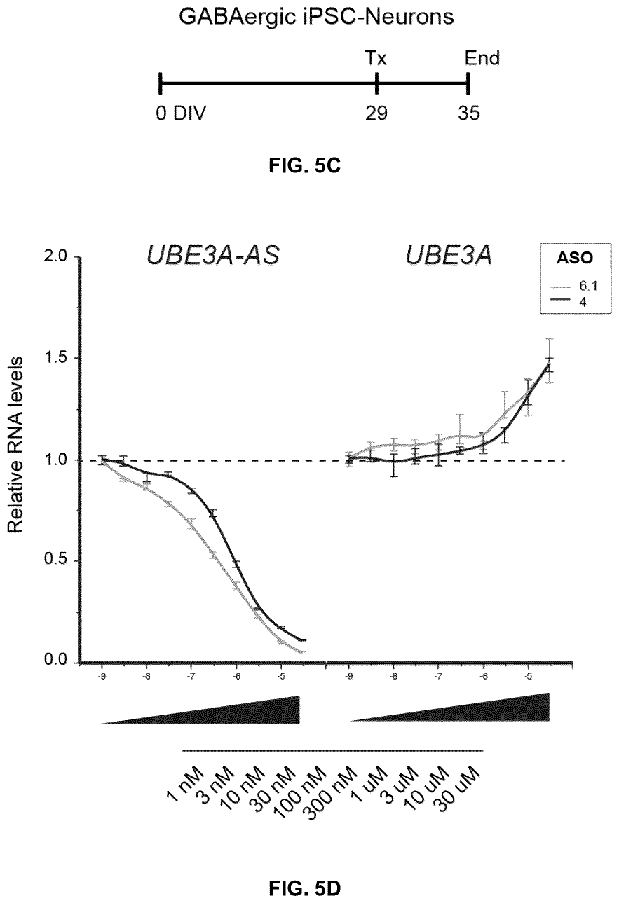

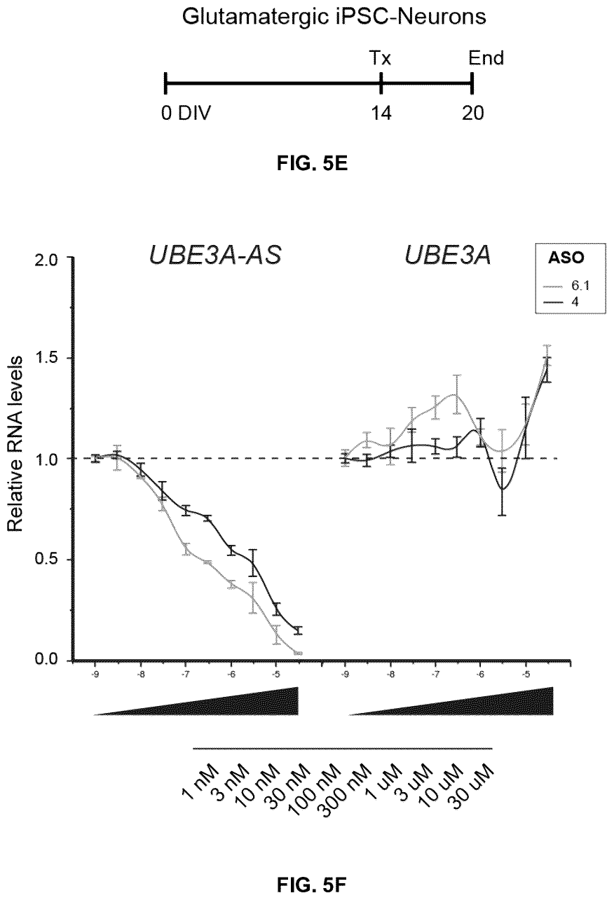

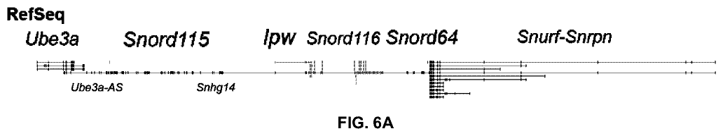

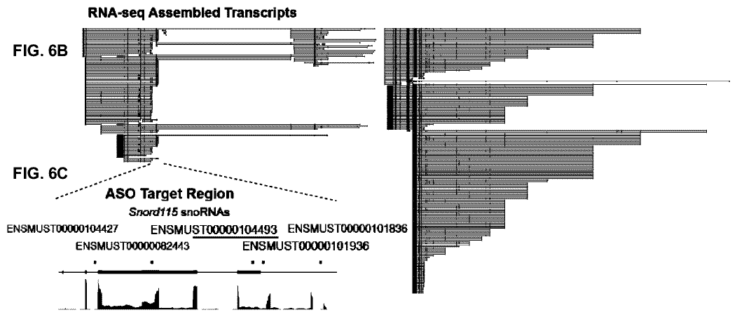

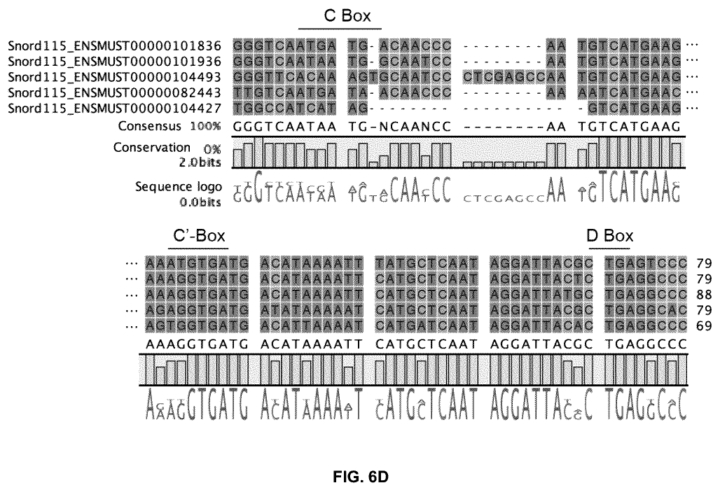

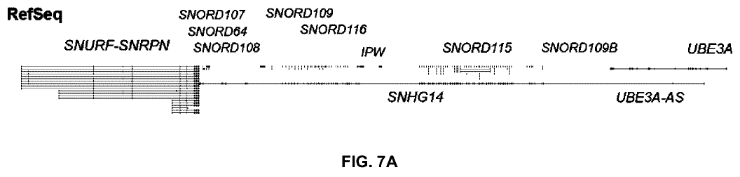

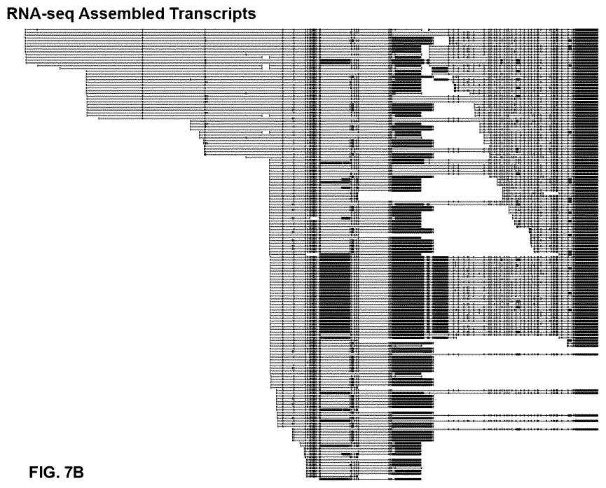

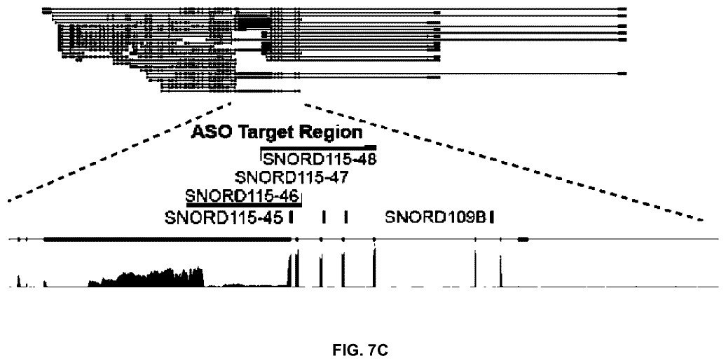

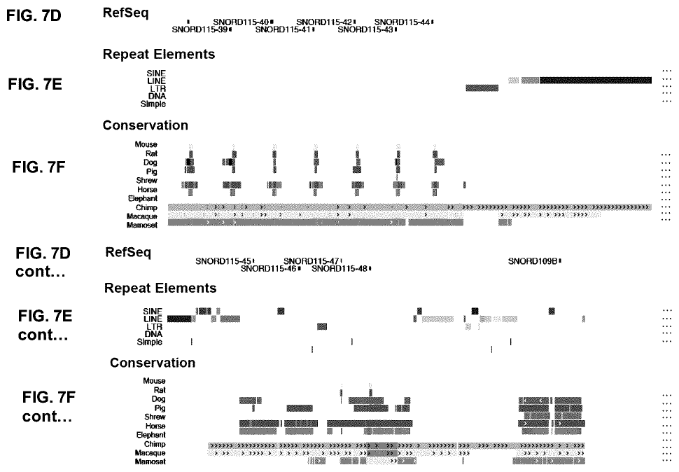

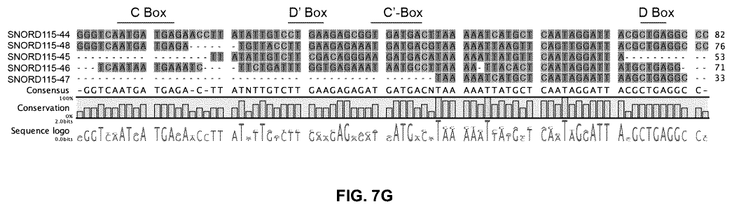

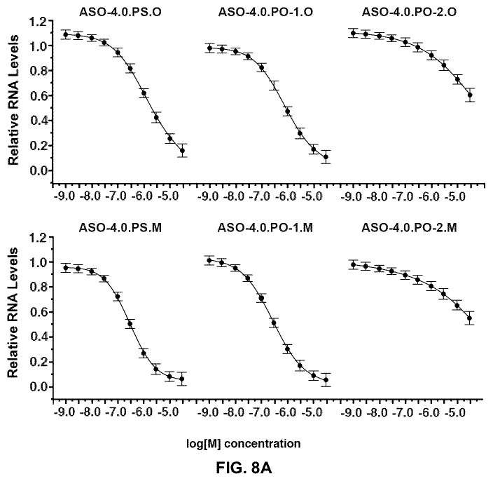

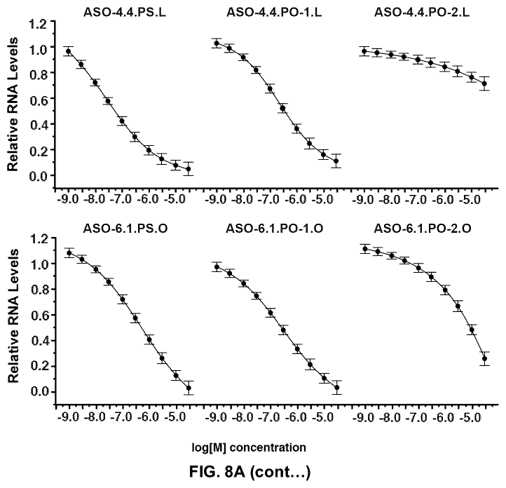

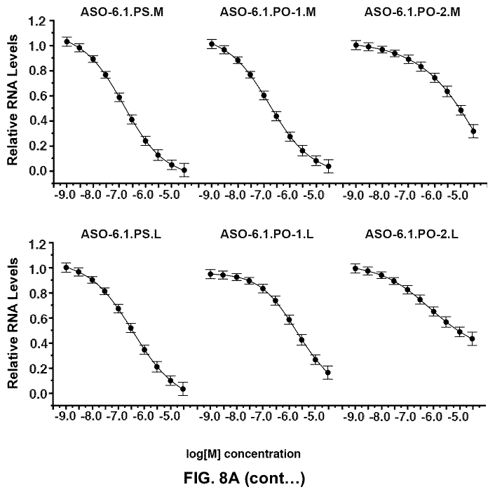

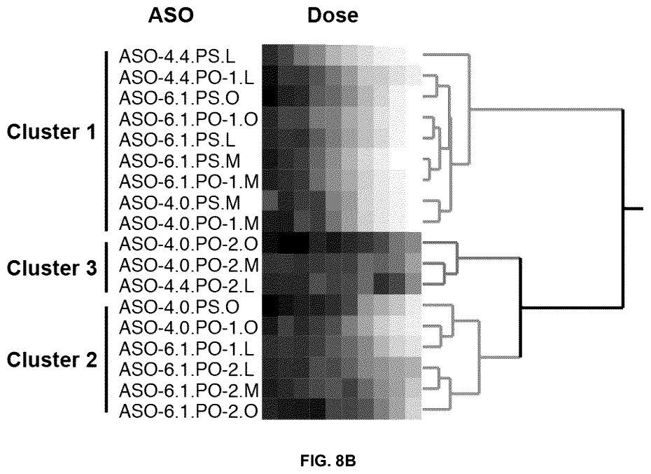

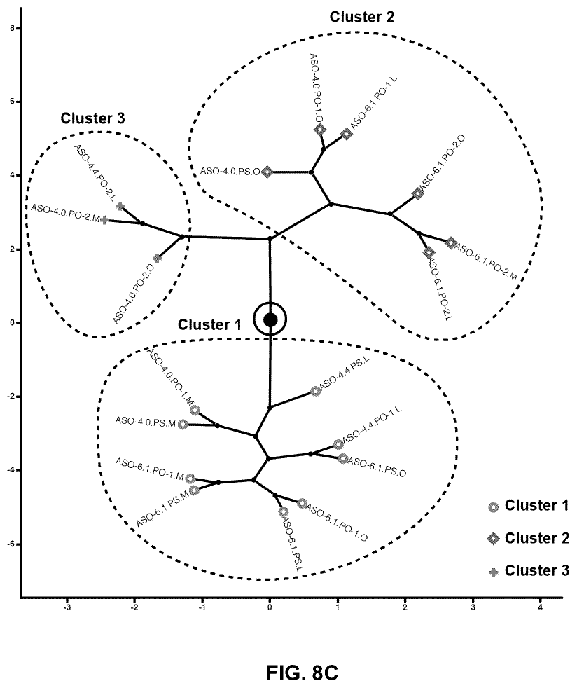

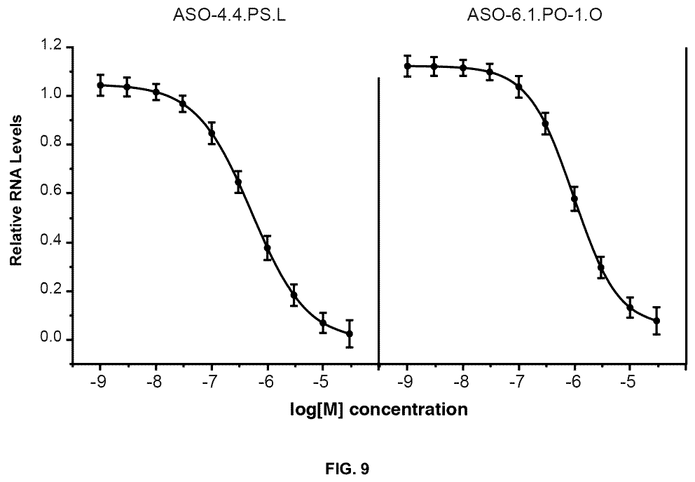

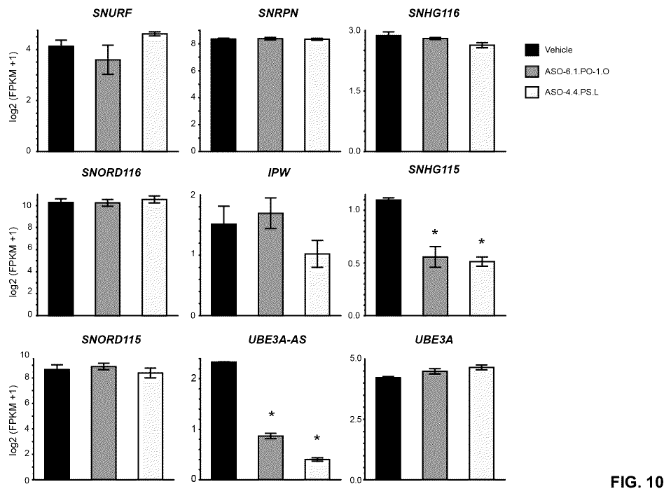

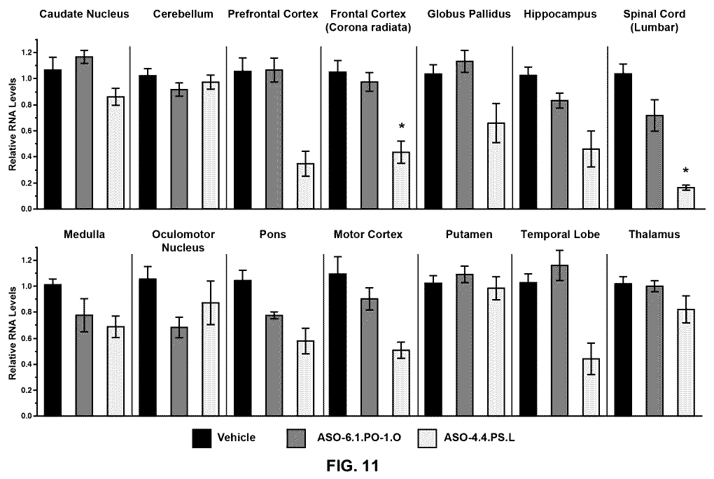

Additional ASOs targeting the 5′-end of UBE3A-AS were then designed to optimize the target sequences of ASO-4 (ASO-4.1, ASO-4.2, ASO-4.3, and ASO-4.4) as well as two other target regions, ASO-3 (ASO-3.1 and ASO-3.2) and ASO-6 (ASO-6.1) (Table 11). Additionally, ASO-4 was manufactured at two different vendors for comparative purposes (ASO-4.S, Sigma; ASO-4.I, Integrated DNA Technologies). Human iPSC-derived neurons (GABAergic) were treated at 14 DIV with a 5-point ½ log dose curve of ASO-3.1, ASO-3.2, ASO-4.S, ASO-4.I, ASO-4.1, ASO-4.2, ASO-4.3, ASO-4.4, and ASO-6.1 [30 nM, 100 nM, 300 nM, 1 μM (n=6)]. At 20 DIV, the IC₅₀ and Eₘₐₓ of each ASO was estimated as described above (FIG. 5A-B and Table 12). The dose response curves were similar among ASOs (Parallelism test: F_{(16.513)}=1.6, p=0.06), with ASO-4 and ASO-6.1 having the highest relative potency (Table 13). No significant difference was observed between ASO-4.S and ASO-4.I.

The potency of ASO-4 and ASO-6.1 was further examined in iPSC-derived neurons at a later time point in differentiation. GABAergic iPSC-derived neurons were treated at 29 DIV with a 10-point ½ log dose response curve of ASO-4 and ASO-6.1 [1 nM, 3 nM, 10 nM, 30 nM, 100 nM, 300 nM, 1 μM, 3 μM, 10 μM, and 30 μM (n=3)]. At 35 DIV, the IC₅₀ and Eₘₐₓ of each ASO was estimated as described above (FIG. 5C-D and Table 14). The dose response curves of ASO-4 and ASO-6.1 were not similar (Parallelism test: F_{(3.172)}=22.7, p<0.0001). An equivalence test indicated that ASO-4 and ASO-6.1 had equivalent potencies but different Eₘₐₓ values [ASO-6.1/ASO-4 ratio: IC₅₀=1.03 (Lower confidence limit=1.0; Upper confidence limit=1.1); Eₘₐₓ=−1.3 (Lower confidence limit=−2.6; Upper confidence limit=−0.08)], with ASO-6.1 having the largest inhibition of UBE3A-AS levels. The effect of ASO-4 and ASO-6.1 on UBE3A RNA levels was similar, with each treatment increasing RNA levels in dose dependent manner (FIG. 5D).

ASO-4 and ASO-6.1 were also examined in glutamatergic iPSC-derived neurons. Glutamatergic iPSC-derived neurons were treated at 14 DIV with a 10-point ½ log dose response curve of ASO-4 and ASO-6.1 [1 nM, 3 nM, 10 nM, 30 nM, 100 nM, 300 nM, 1 μM, 3 μM, 10 μM, and 30 μM (n=3)]. At 20 DIV, the IC₅₀ and Eₘₐₓ of each ASO was estimated as described above (FIG. 5E-F and Table 15). The dose response curves of ASO-4 and ASO-6.1 were similar and not significantly different (Parallelism test: F_{(3.165)}=1.9, p=0.1), with ASO-6.1 having the highest relative potency (Table 16). As expected, ASO-4 and ASO-6.1 increased UBE3A RNA levels in a dose dependent manner (FIG. 5F); however, there was a high degree of variation for each concentration that was not attributable to treatment (R²=0.17).

### Conclusions

Towards developing a therapy for AS, experiments were conducted to determine whether ASOs targeting a specific region inhibit Ube3a-AS/UBE3A-AS and reactivate expression of the paternal Ube3a/UBE3A allele in mouse and human neurons. Altogether, findings show that ASOs targeting this region in mouse and human neurons have potent antisense activity and reverse imprinting of Ube3a/UBE3A.

Two of the three ASOs (ASO-1.1 and ASO-3.1) targeting Ube3a-AS reactivated expression of the paternal Ube3a allele in mouse neurons to a level similar to that achieved by the optimal concentration of Topotecan (300 nM).

Likewise, each of the human-specific ASOs significantly reduced the steady state RNA levels of UBE3A-AS in human iPSC-derived neurons, with higher concentrations of ASO-4 and ASO-6.1 almost completely abolishing expression of UBE3A-AS. Given that ASO-4 and ASO-6.1 target regions that are 100% conserved between human and macaque, the efficacy of these ASOs can be examined in vivo in either Cynomolgus or Rhesus macaque. Unlike Topotecan, ASO-4 has a small, if any, effect on the upstream SNORD116, IPW, SNORD115, or SNORD109A/B RNAS, consistent with the notion that the ASO terminates transcription at or downstream of the target region.

Low concentrations (3 nM) of ASO-4 and ASO-6.1 significantly reduced UBE3A-AS RNA levels; however, higher concentrations (≥100 nM) of ASO were necessary to increase UBE3A RNA levels. This may reflect a certain threshold required for UBE3A-AS to inhibit transcription of UBE3A, or a lag between the time that inactivation of UBE3A-AS leads to reactivation of paternal UBE3A, or the sensitivity of the assay used to quantify UBE3A RNA levels.

Collectively, findings suggest that ASOs targeting a candidate region in UBE3A-AS almost completely abolishes imprinting of UBE3A in neurons and reveals at least two ASOs for future clinical development.

Derivatives of ASO-4 and ASO-6.1 that are comprised of different RNA modifications [2′-hydroxymethyl (2′-OMe), 2′-methoxy-ethyl 2′-MOE, and locked nucleic acid (LNA)] and backbones [phosphorothioate (PS) and phosphodiester (PO)] have also been designed (Table 17).

TABLE 6
Mouse Ube3a-AS Oligonucleotides

| | RNA | RNA | DNA | | | |
|---|---|---|---|---|---|---|
| | Modifi- | Back- | Back- | Design | | |
| ASO | cation | bone | bone | (5′-3′) | Sequence | SEQ ID |
| ASO-B | 2′-OMe | PS | PS | 5-10-5 | C^{O}*C^{O}*A^{O}*G^{O}*C^{O}*c*t*t*g*t*t*g*g*a*t*A^{O}*U^{O}*C^{O}* | SEQ ID NO: 358 |
| | | | | | A^{O}*U^{O} | |
| ASO-1.1 | 2′-OMe | PS | PS | 5-10-5 | C^{O}*C^{O}*A^{O}*C^{O}*A^{O}*t*t*t*c*c*t*c*t*c*a*U^{O}*G^{O}*G^{O}* | SEQ ID NO: 359 |
| | | | | | A^{O}*A^{O} | |
| ASO-1.2 | 2′-OMe | PS | PS | 5-10-5 | G^{O}*A^{O}*G^{O}*U^{O}*G^{O}*t*t*t*g*c*a*a*a*c*c*A^{O}*A^{O}*U^{O}* | SEQ ID NO: 360 |
| | | | | | G^{O}*U^{O} | |
| ASO-3.1 | 2′-OMe | PS | PS | 5-10-5 | U^{O}*G^{O}*U^{O}*U^{O}*U^{O}*c*t*t*t*g*g*t*g*a*t*U^{O}*C^{O}*U^{O}* | SEQ ID NO: 361 |
| | | | | | G^{O}*C^{O} | |

Capital letter, RNA; lower-case letter, DNA; O, 2′-OMe; PS & *, phosphorothioate

TABLE 7
Human UBE3A-AS Oligonucleotides

| | RNA | RNA | DNA | | | |
|---|---|---|---|---|---|---|
| | Modifi- | Back- | Back- | Design | | |
| ASO | cation | bone | bone | (5′-3′) | Sequence | SEQ ID |
| ASO-1 | 2′-OMe | PS | PS | 5-10-5 | U^{O}*A^{O}*G^{O}*A^{O}*G^{O}*g*t*g*a*a*g*g*c*c*a*G^{O}*G^{O}*C^{O}* | SEQ ID NO: 362 |
| | | | | | A^{O}*C^{O} | |
| ASO-2 | 2′-OMe | PS | PS | 5-10-5 | G^{O}*U^{O}*A^{O}*C^{O}*U^{O}*c*t*t*c*c*t*c*a*g*t*C^{O}*A^{O}*U^{O}* | SEQ ID NO: 363 |
| | | | | | C^{O}*C^{O} | |
| ASO-3^{C} | 2′-OMe | PS | PS | 5-10-5 | U^{O}*G^{O}*U^{O}*C^{O}*A^{O}*g*t*t*t*c*t*c*c*c*t*G^{O}*A^{O}*A^{O}* | SEQ ID NO: 364 |
| | | | | | C^{O}*A^{O} | |
| ASO-4^{C} | 2′-OMe | PS | PS | 5-10-5 | U^{O}*A^{O}*G^{O}*A^{O}*A^{O}*t*g*g*c*a*c*a*t*c*t*C^{O}*U^{O}*U^{O}* | SEQ ID NO: 365 |
| | | | | | G^{O}*G^{O} | |
| ASO-5^{C} | 2′-OMe | PS | PS | 5-10-5 | G^{O}*U^{O}*U^{O}*U^{O}*U^{O}*c*t*t*c*c*t*c*c*a*c*A^{O}*G^{O}*U^{O}* | SEQ ID NO: 366 |
| | | | | | C^{O}*U^{O} | |
| ASO-6^{C} | 2′-OMe | PS | PS | 5-10-5 | C^{O}*U^{O}*G^{O}*G^{O}*U^{O}*g*t*c*a*a*c*a*a*g*c*C^{O}*A^{O}*A^{O}* | SEQ ID NO: 367 |
| | | | | | A^{O}*G^{O} | |

Abbreviations: ^{C}, conserved with macaque & non-polymorphic; capital letter, RNA nucleotide; lower-case letter, DNA nucleotide; 0, 2′-OMe; PS & *, phosphorothioate

TABLE 8
Analysis of Human ASOs on UBE3A-AS and UBE3A RNA levels

| | | | | | |
|---|---|---|---|---|---|
| ASO 1 | ASO 2 | Difference | Lower CI | Upper CI | Adj. P |

UBE3A-AS

| | | | | | |
|---|---|---|---|---|---|
| ASO-C | ASO-2 | 0.89 | 0.82 | 0.97 | <.0001 |
| ASO-R | ASO-2 | 0.87 | 0.80 | 0.95 | <.0001 |
| ASO-C | ASO-4 | 0.87 | 0.80 | 0.95 | <.0001 |
| ASO-R | ASO-4 | 0.85 | 0.78 | 0.93 | <.0001 |
| ASO-C | ASO-6 | 0.83 | 0.75 | 0.90 | <.0001 |
| ASO-R | ASO-6 | 0.81 | 0.74 | 0.89 | <.0001 |
| ASO-C | ASO-3 | 0.79 | 0.71 | 0.86 | <.0001 |
| ASO-R | ASO-3 | 0.77 | 0.70 | 0.85 | <.0001 |
| ASO-C | ASO-5 | 0.71 | 0.63 | 0.78 | <.0001 |
| ASO-R | ASO-5 | 0.69 | 0.62 | 0.77 | <.0001 |
| ASO-C | Topo | 0.66 | 0.59 | 0.73 | <.0001 |
| ASO-R | Topo | 0.64 | 0.57 | 0.72 | <.0001 |
| ASO-C | ASO-1 | 0.51 | 0.43 | 0.58 | <.0001 |
| ASO-R | ASO-1 | 0.49 | 0.41 | 0.56 | <.0001 |
| ASO-1 | ASO-2 | 0.38 | 0.31 | 0.46 | <.0001 |
| ASO-1 | ASO-4 | 0.36 | 0.29 | 0.44 | <.0001 |
| ASO-1 | ASO-6 | 0.32 | 0.25 | 0.40 | <.0001 |
| ASO-1 | ASO-3 | 0.28 | 0.21 | 0.36 | <.0001 |
| Topo | ASO-2 | 0.23 | 0.16 | 0.31 | <.0001 |
| Topo | ASO-4 | 0.21 | 0.14 | 0.29 | <.0001 |
| ASO-1 | ASO-5 | 0.20 | 0.13 | 0.28 | <.0001 |
| ASO-5 | ASO-2 | 0.18 | 0.11 | 0.26 | <.0001 |
| Topo | ASO-6 | 0.17 | 0.10 | 0.24 | 0.0002 |
| ASO-5 | ASO-4 | 0.16 | 0.09 | 0.24 | 0.0003 |
| ASO-1 | Topo | 0.15 | 0.08 | 0.23 | 0.0004 |
| Topo | ASO-3 | 0.13 | 0.06 | 0.20 | 0.0018 |
| ASO-5 | ASO-6 | 0.12 | 0.04 | 0.20 | 0.0035 |
| ASO-3 | ASO-2 | 0.10 | 0.03 | 0.18 | 0.0111 |
| ASO-3 | ASO-4 | 0.08 | 0.01 | 0.16 | 0.0360 |
| ASO-5 | ASO-3 | 0.08 | 0.00 | 0.15 | 0.0381 |
| ASO-6 | ASO-2 | 0.06 | −0.01 | 0.14 | 0.11 |
| Topo | ASO-5 | 0.05 | −0.02 | 0.13 | 0.18 |
| ASO-6 | ASO-4 | 0.04 | −0.03 | 0.12 | 0.27 |
| ASO-3 | ASO-6 | 0.04 | −0.03 | 0.12 | 0.28 |
| ASO-4 | ASO-2 | 0.02 | −0.06 | 0.09 | 0.58 |
| ASO-C | ASO-R | 0.02 | −0.06 | 0.09 | 0.64 |

UBE3A

| | | | | | |
|---|---|---|---|---|---|
| ASO-4 | ASO-C | 0.30 | 0.16 | 0.44 | 0.0004 |
| ASO-4 | ASO-R | 0.29 | 0.14 | 0.45 | 0.001 |
| ASO-2 | ASO-C | 0.21 | 0.09 | 0.34 | 0.002 |
| ASO-2 | ASO-R | 0.21 | 0.07 | 0.35 | 0.006 |
| ASO-1 | ASO-C | 0.18 | 0.06 | 0.31 | 0.007 |
| Topo | ASO-C | 0.18 | 0.04 | 0.32 | 0.01 |
| ASO-1 | ASO-R | 0.18 | 0.04 | 0.32 | 0.02 |
| Topo | ASO-R | 0.18 | 0.03 | 0.33 | 0.02 |
| ASO-4 | ASO-3 | 0.17 | 0.03 | 0.31 | 0.02 |
| ASO-5 | ASO-C | 0.16 | 0.04 | 0.29 | 0.01 |
| ASO-6 | ASO-C | 0.16 | 0.04 | 0.29 | 0.01 |
| ASO-5 | ASO-R | 0.16 | 0.02 | 0.30 | 0.03 |
| ASO-6 | ASO-R | 0.16 | 0.02 | 0.30 | 0.03 |
| ASO-4 | ASO-6 | 0.13 | −0.007 | 0.27 | 0.06 |
| ASO-4 | ASO-5 | 0.13 | −0.007 | 0.27 | 0.06 |
| ASO-3 | ASO-C | 0.13 | 0.00 | 0.26 | 0.04 |
| ASO-3 | ASO-R | 0.13 | −0.015 | 0.27 | 0.08 |
| ASO-4 | Topo | 0.11 | −0.04 | 0.27 | 0.1 |
| ASO-4 | ASO-1 | 0.11 | −0.03 | 0.25 | 0.1 |
| ASO-2 | ASO-3 | 0.08 | −0.04 | 0.21 | 0.2 |
| ASO-4 | ASO-2 | 0.08 | −0.06 | 0.22 | 0.2 |
| ASO-1 | ASO-3 | 0.05 | −0.07 | 0.18 | 0.4 |
| Topo | ASO-3 | 0.05 | −0.09 | 0.19 | 0.4 |
| ASO-2 | ASO-6 | 0.05 | −0.08 | 0.18 | 0.4 |
| ASO-2 | ASO-5 | 0.05 | −0.08 | 0.17 | 0.4 |
| ASO-5 | ASO-3 | 0.03 | −0.09 | 0.16 | 0.6 |
| ASO-6 | ASO-3 | 0.03 | −0.09 | 0.16 | 0.6 |
| ASO-2 | Topo | 0.03 | −0.1 | 0.17 | 0.7 |
| ASO-2 | ASO-1 | 0.03 | −0.01 | 0.16 | 0.6 |
| ASO-1 | ASO-6 | 0.02 | −0.1 | 0.15 | 0.7 |
| Topo | ASO-6 | 0.02 | −0.1 | 0.16 | 0.8 |
| ASO-1 | ASO-5 | 0.02 | −0.1 | 0.15 | 0.7 |
| Topo | ASO-5 | 0.02 | −0.1 | 0.16 | 0.8 |
| ASO-R | ASO-C | 0.00 | −0.1 | 0.14 | 0.9 |
| ASO-5 | ASO-6 | 0.00 | −0.1 | 0.13 | 0.9 |
| ASO-1 | Topo | 0.00 | −0.1 | 0.14 | 1.00 |

Abbreviations:
ASO-C, ASO-control;
Topo, Topotecan;
Adj., Adjusted;
CI, 95% confidence interval

TABLE 9
IC₅₀ and Eₘₐₓ of ASO-4 and Topotecan

| Treatment | IC₅₀ Estimate (M) | IC₅₀ 95% CI (M) | | Eₘₐₓ Estimate | Eₘₐₓ 95% CI | | 30 μM (Mean) |
|---|---|---|---|---|---|---|---|
| ASO-4 | 6.13E−07 | 3.47E−07 | 1.08E−06 | −0.06 | −0.23 | 0.10 | 0.09 |
| Topo | 3.37E−08 | 1.85E−08 | 6.14E−08 | 0.26 | 0.20 | 0.32 | 0.21 |

Full model parameter estimates from 4-parameter logistic regression model (Hill).
IC₅₀ and confidence intervals represent molar concentration.
Eₘₐₓ and 30 uM values represent normalized UBE3A-AS RNA levels relative to vehicle.

TABLE 10
Analysis of ASO-4 and Topotecan on UBE3A, SNORD116,
SNORD115, SNORD109A/B, and IPW RNA Levels

| Treatment | RNA | DF | DFDen | F Ratio | FDR |
|---|---|---|---|---|---|
| ASO-4 | UBE3A | 9 | 108 | 16.5 | <0.0001 |
| | SNORD109A/B | 9 | 104.9 | 2.6 | 0.01 |
| | SNORD115 | 9 | 108 | 4.0 | 0.0002 |
| | SNORD116 | 9 | 108 | 1.74 | 0.09 |
| | IPW | 9 | 108 | 4.1 | 0.0002 |
| Topotecan | UBE3A | 9 | 29 | 5.6 | 0.0002 |
| | SNORD109A/B | 9 | 29 | 28.2 | <0.0001 |
| | SNORD115 | 9 | 29 | 4.60 | 0.001 |
| | SNORD116 | 9 | 29 | 7.12 | <0.0001 |
| | IPW | 9 | 29 | 49.8 | <0.0001 |

Least squares linear regression.
Abbreviations:
DF, degrees of freedom;
DFDen, degrees of freedom density

TABLE 11
Optimized Human UBE3A-AS Antisense Oligonucleotides

| | RNA | RNA | DNA | | | |
|---|---|---|---|---|---|---|
| | Modifi- | Back- | Back- | Design | | |
| ASO | cation | bone | bone | (5′-3′) | Sequence | SEQ ID |
| ASO-3.1^{C} | 2'-OMe | PS | PS | 4-10-5 | G^{O}*U^{O}*U^{O}*G^{O}*a*g*t*g*g*t*g*t*c*a*G^{O*}U^{O}* | SEQ ID NO: 368 |
| | | | | | U^{O*}U^{O*}C^{O} | |
| ASO-3.2^{C} | 2'-OMe | PS | PS | 4-10-4 | U^{O}*U^{O}*G^{O}*A^{O}*g*t*g*g*t*g*t*c*a*g*U^{O*}U^{O}* | SEQ ID NO: 369 |
| | | | | | U^{O*}C^{O} | |
| ASO-6.1^{C} | 2'-OMe | PS | PS | 4-10-4 | C^{O}*U^{O}*G^{O}*G^{O}*t*g*t*c*a*a*c*a*a*g*C^{O*}C^{O}* | SEQ ID NO: 370 |
| | | | | | A^{O*}A^{O} | |
| ASO-4.1^{C} | 2'-OMe | PS | PS | 5-10-5 | A^{O}*U^{O}*A^{O}*G^{O}*A^{O}*a*t*g*g*c*a*c*a*t*c*U^{O*} | SEQ ID NO: 371 |
| | | | | | C^{O}*U^{O*}U^{O*}G^{O} | |
| ASO-4.2^{C} | 2'-OMe | PS | PS | 4-10-5 | A^{O}*G^{O}*A^{O}*A^{O}*t*g*g*c*a*c*a*t*c*t*C^{O*}U^{O}* | SEQ ID NO: 372 |
| | | | | | U^{O*}G^{O*}G^{O} | |
| ASO-4.3^{C} | 2'-OMe | PS | PS | 4-10-5 | U^{O}*A^{O}*G^{O}*A^{O}*a*t*t*g*c*a*c*a*t*c*U^{O*}C^{O}* | SEQ ID NO: 373 |
| | | | | | U^{O*}U^{O*}G^{O} | |
| ASO-4.4^{C} | 2'-OMe | PS | PS | 4-10-4 | A^{O}*G^{O}*A^{O}*A^{O}*t*g*g*c*a*c*a*t*c*t*C^{O*}U^{O}* | SEQ ID NO: 374 |
| | | | | | U^{O*}G^{O} | |

^{C}, conserved with macaque & non-polymorphic; capital letter, RNA nucleotide; lower-case letter, DNA nucleotide; O, 2'-OMe; PS & * phosphorothioate

TABLE 12
IC₅₀ and Eₘₐₓ of Optimized ASO Target Sequences

| Group | IC₅₀ Estimate | IC₅₀ 95% CI (M) | | 3 μM (Mean) |
|---|---|---|---|---|
| 6.1 | 5.20E−07 | 3.33E−07 | 8.11E−07 | 0.23 |
| 4.0 | 1.06E−06 | 9.31E−07 | 1.21E−06 | 0.29 |
| 4.2 | 1.08E−06 | 8.62E−07 | 1.35E−06 | 0.31 |
| 3.2 | 1.88E−06 | 1.39E−06 | 2.56E−06 | 0.44 |
| 4.3 | 2.03E−06 | 1.67E−06 | 2.47E−06 | 0.40 |
| 4.4 | 2.11E−06 | 1.73E−06 | 2.59E−06 | 0.44 |
| 4.1 | 2.27E−06 | 1.92E−06 | 2.68E−06 | 0.42 |
| 3.1 | 2.98E−06 | 2.45E−06 | 3.62E−06 | 0.51 |

Full model parameter estimates from 3-parameter logistic regression model. IC₅₀ and confidence intervals represent molar concentration. Eₘₐₓ (3 uM) values represent normalized UBE3A-AS RNA levels relative to vehicle.

TABLE 13
Relative Potency of Optimized ASOs

| ASO | IC₅₀ (M) | Relative Potency | Std. Error |
|---|---|---|---|
| ASO 3.1 | 2.81E−06 | 0.53 | 0.059 |
| ASO 3.2 | 1.85E−06 | 0.81 | 0.086 |
| ASO 4.1 | 2.25E−06 | 0.66 | 0.072 |
| ASO 4.2 | 1.24E−06 | 1.21 | 0.13 |
| ASO 4.3 | 1.96E−06 | 0.76 | 0.081 |
| ASO 4.4 | 2.04E−06 | 0.73 | 0.079 |
| ASO 6.1 | 7.20E−07 | 2.07 | 0.21 |
| ASO 4.I | 8.28E−07 | 1.80 | 0.19 |
| ASO 4.S | 1.49E−06 | 1 | 0 |

Parallel model parameter estimates from 3-parameter logistic regression model. Potency represents molar concentration.
Abbreviations: M, molar; Std. Error, standard error of mean.

TABLE 14
IC₅₀ and Eₘₐₓ of ASO-4 and ASO-6.1 in GABAergic iPSC Neurons

| ASO | IC₅₀ Estimate | IC₅₀ 95% CI (M) | | Eₘₐₓ Estimate | Eₘₐₓ 95% CI | | 30 μM (Mean) |
|---|---|---|---|---|---|---|---|
| ASO-4 | 7.77E−07 | 6.86E−07 | 8.79E−07 | 0.08 | 0.05 | 0.11 | 0.11 |
| ASO-6.1 | 5.17E−07 | 3.41E−07 | 7.82E−07 | −0.11 | −0.22 | 0.01 | 0.06 |

Full model parameter estimates from 4-parameter logistic regression model (Hill).
IC₅₀ and confidence intervals represent molar concentration.
Eₘₐₓ and 30 uM values represent normalized UBE3A-AS RNA levels relative to vehicle.

TABLE 15
IC₅₀ and Eₘₐₓ of ASO-4 and ASO-6.1 in Glutamatergic iPSC Neurons

| ASO | IC₅₀ Estimate | IC₅₀ 95% CI (M) | | Eₘₐₓ Estimate | Eₘₐₓ 95% CI | | 30 μM (Mean) |
|---|---|---|---|---|---|---|---|
| ASO-4 | 1.21E−04 | 1.12E−13 | 1.32E+05 | −1.45 | −9.01 | 6.12 | 0.17 |
| ASO-6.1 | 2.44E−07 | 2.39E−08 | 2.50E−06 | −0.27 | −1.24 | 0.70 | 0.04 |

Full model parameter estimates from 4-parameter logistic regression model (Hill).
IC₅₀ and confidence intervals represent molar concentration.
Eₘₐₓ and 30 uM values represent normalized UBE3A-AS RNA levels relative to vehicle.

TABLE 16
Relative Potency of ASO-4 and ASO-6.1 in Glutamatergic Neurons

| ASO | IC₅₀ (M) | Relative Potency | Std. Error |
|---|---|---|---|
| ASO-4 | 3.06E−06 | 1 | 0 |
| ASO-6.1 | 7.8E−07 | 3.89 | 0.72 |

Parallel model parameter estimates from 4 Parameter logistic regression model.
Abbreviations:
M, molar

TABLE 17
Derivatives of ASO-4 and ASO-6.1

| | RNA | | PO | Design | | |
|---|---|---|---|---|---|---|
| ASO | Mod. | Backbone | linkages | (5′-3′) | Sequence (5′-3′) | SEQ ID |
| ASO-4.0.PS.O | OMe | PS | 0 | 5-10-5 | U^{O}*A^{O}*G^{O}*A^{O}*A^{O}*t*g*g*c*a*c*a*t*c*t*C^{O}*U^{O}*U^{O}*Gº*Gº | SEQ ID NO: 375 |
| ASO-4.0.PO-1.O | OMe | PS/PO | 2 | 5-10-5 | U^{O}*A^{O}*G^{O}*A^{O}*A^{O}-t*g*g*c*a*c*a*t*c*t-C^{O}*U^{O}*U^{O}*G^{O}*G^{O} | SEQ ID NO: 376 |
| ASO-4.0.PO-2.O | OMe | PS/PO | 0 | 5-10-5 | U^{O}*A^{O}-G^{O}*Aº-A^{O}*t-g*g-c*a-c*a-t*c-t*C^{O}-U^{O}*U^{O}-G^{O}*G^{O} | SEQ ID NO: 377 |
| ASO-4.0.PS.M | MOE | PS | 0 | 5-10-5 | T^{M}*A^{M}*G^{M}*A^{M}*A^{M}*t*g*g*c*a*c*a*t*c*t*5mC^{M}*T^{M}*T^{M}*G^{M}*G^{M} | SEQ ID NO: 378 |
| ASO-4.0.PO-1.M | MOE | PS/PO | 2 | 5-10-5 | T^{M}*A^{M}*G^{M}*A^{M}*A^{M}-t*g*g*c*a*c*a*t*c*t-5mC^{M}*T^{M}*T^{M}*G^{M}*G^{M} | SEQ ID NO: 379 |
| ASO-4.0.PO-2.M | MOE | PS/PO | 9 | 5-10-5 | T^{M}*A^{M}-G^{M}*A^{M}-A^{M}*t-g*g-c*a-c*a-t*c-t*5mC^{M}-T^{M}*T^{M}-G^{M}*G^{M} | SEQ ID NO: 380 |
| ASO-4.4.PS.L | LNA | PS | 0 | 3-11-4 | A*G^{L}*A^{L}*a*t*g*g*c*a*c*a*t*c*t*5mC^{L}*T^{L}*T^{L}*G^{L} | SEQ ID NO: 381 |
| ASO-4.4.PO-1.L | LNA | PS/PO | 2 | 3-11-4 | A^{L}*G^{L}*A^{L}-a*t*g*g*c*a*c*a*t*c*t-5mC^{L}*T^{L}*T^{L}*G^{L} | SEQ ID NO: 382 |
| ASO-4.4.PO-2.L | LNA | PS/PO | 8 | 3-11-4 | A^{L}*G^{L}-A^{L}*a-t*g-g*c-a*c-a*t-c*t-5mC^{L}*T^{L}-T^{L}*G^{L} | SEQ ID NO: 383 |
| ASO-6.1.PS.O | OMe | PS | 0 | 4-10-4 | C^{O}*U^{O}*G^{O}*G^{O}*t*g*t*c*a*a*c*a*a*g*C^{O}*C^{O}*A^{O}*Aº | SEQ ID NO: 384 |
| ASO-6.1.PO-1.O | OMe | PS/PO | 2 | 4-10-4 | C^{O}*U^{O}*G^{O}*G^{O}-t*g*t*c*a*a*c*a*a*g-C^{O}*C^{O}*A^{O}*A^{O} | SEQ ID NO: 385 |
| ASO-6.1.PO-2.O | OMe | PS/PO | 8 | 4-10-4 | C^{O}*U^{O}-G^{O}*G^{O}-t*g-t*c-a*a-c*a-a*g-C^{O}*C^{O}-A^{O}*A^{O} | SEQ ID NO: 386 |
| ASO-6.1.PS.M | MOE | PS | 0 | 4-10-4 | 5mC^{M}*T^{M}*G^{M}*G^{M}*t*g*t*c*a*a*c*a*a*g*5mCM*5mC^{M}*A^{M}*A^{M} | SEQ ID NO: 387 |
| ASO-6.1.PO-1.M | MOE | PS/PO | 2 | 4-10-4 | 5mC^{M}*T^{M}*G^{M}*G^{M}-t*g*t*c*a*a*c*a*a*g-5mC^{M}*5mC^{M}*A^{M}*A^{M} | SEQ ID NO: 388 |
| ASO-6.1.PO-2.M | MOE | PS/PO | 8 | 4-10-4 | 5mC^{M}*T^{M}-G^{M}*G^{M}-t*g-t*c-a*a-c*a-a*g-5mC^{M}*5mC^{M}-A^{M}*A^{M} | SEQ ID NO: 389 |
| ASO-6.1.PS.L | LNA | PS | 0 | 3-10-4 | T^{L}*G^{L}*G^{L}*t*g*t*c*a*a*c*a*a*g*5mC^{L}*5mC^{L}*A^{L}*A^{L} | SEQ ID NO: 390 |
| ASO-6.1.PO-1.L | LNA | PS/PO | 2 | 3-10-4 | T^{L}*G^{L}*G^{L}-t*g*t*c*a*a*c*a*a*g-5mC^{L}*5mC^{L}*A^{L}*A^{L} | SEQ ID NO: 391 |
| ASO-6.1.PO-2.L | LNA | PS/PO | 8 | 3-10-4 | T^{L}*G^{L}-G^{L}*t-g*t-c*a-a*c-a*a-g*5mC^{L}-5mC^{L}-A^{L}*A^{L} | SEQ ID NO: 392 |

Capital letter, RNA; lower-case letter, DNA. 5mC, 5-methylcytosine. Superscript: O, 2′-OMe; M, 2′-MOE; L, LNA. PS & *, phosphorothioate; PO & -, phosphodiester

### Materials and Methods

### Antisense Oligonucleotide Design

Antisense oligonucleotides (ASOs) were designed using Soligo (Software for Statistical Folding of Nucleic Acids and Studies of Regulatory RNAs). Briefly, candidate ASOs (20-18 mer) with the lowest binding site disruption energy and free binding energy were identified for each target sequence and then inspected for motifs with increased effectiveness. ASOs were further filtered based on accessibility within predicted lowest free energy centroid secondary structure of target sequence generated by Soligo. In some instances, secondary structure models were compared using lowest free energy structures generated by RNAfold and Mfold.

Human ASOs were filtered using the following criteria: 1) target sequence was polymorphic [dbSNP138, dbSNP150, and 1000 Genomes Phase 3 Integrated Variant Calls (SNV, INDEL, and SV)]; 2) target sequence was not 100% conserved with Rhesus and Cynomolgus macaque; 3) target sequence was located upstream of retained Snord115/SNORD115 snoRNA (per exon). Remaining ASOs were then ranked by free energy (<=−8 kcal/mol), average unpaired probability for target site nucleotides, binding site disruption energy (low>high), location within secondary structure (Ensembl Centroid), and presence/absence of sequence motifs associated with high/low effectiveness.

### Mouse Primary Hippocampal Neurons

Primary cultures of hippocampal neurons were generated from P0-P1 pups (Ube3a^{m+/p+} and Ube3a^{m+/pYFP}) by crossing Ube3a^{m+/pYFP} males with wild-type C57BL/6J females. Genotypes were determined using methods described previously. Briefly, hippocampal neurons were cultured in Neurobasal A medium (Invitrogen, San Diego, CA) supplemented with B27 (Invitrogen) and penicillin/streptomycin (Invitrogen) on 96-well optical bottom plates coated with poly-D-Lysine (152028, Thermo Fisher Scientific) and laminin (23017-01, Thermo Fisher Scientific). Cultures were maintained at 37° C. in 5% CO₂ until use.

### Mouse Neuron Imaging

Mouse primary hippocampal neurons were fixed at 10 DIV (3 days post treatment) with 4% paraformaldehyde. The cultures were then washed twice with 1×PBS, fixed in 4% paraformaldehyde in PBS for 15 min, and then washed three times in 1×PBS. The cells were blocked in 0.3% Triton-X100 in PBS (T-PBS) plus 5% goat or donkey serum for 1-2 hr at room temperature with gentle agitation. Cells were incubated with anti-GFP [Novus Biologicals, NB 600-308 (rabbit)] and anti-NeuN (Millipore, 05-557 (mouse)] antibodies for 24 hr at 4° C. with gentle agitation. Cells were washed 3 times in 0.1% Tween 20 1×PBS for 15 min each and then incubated with anti-rabbit 488 (Jackson ImmunoResearch, 111-545-144) and anti-mouse Cy3 (Jackson ImmunoResearch, 115-165-166) secondary antibodies for 24 hr at 4° C. in the dark. Cells were then washed 4 times in 0.1% Tween 20 1×PBS for 15 min each. Nuclei were labeled using Hoechst stain (Thermo Fisher Scientific) at a dilution of 1:1000 in the third wash.

Plates were imaged using the Cytation 5 and Gen5 Image+ software (BioTek, Winooski, VT). Briefly, a 4× inverted objective was used to generate montage images of each well by acquiring 5×4 autofocused images with overlapping tiles for automatic image stitching. The filters used were DAPI (377,477), GFP (469, 525), and RFP (531, 593). Exposure time and gain were adjusted for each plate using the negative and positive controls. Auto-focus was performed on nuclei (Hoechst stain, DAPI) for each well, with the same focal height used for the GFP and RFP filters. Images were stitched together by Gen5 Image+ software.

Single cell image analysis was performed using IN Cell Developer 6.0 (GE Healthcare Life Sciences, Pittsburgh, PA). Briefly, individual track masks were generated for either nuclei (Hoechst stain, DAPI) or mature neurons (NeuN, RFP) by optimizing inclusion and exclusion parameters based on size and intensity of randomly selected cells in the acquired images. The mean and median intensity values of GFP were then acquired within the boundaries of the selected mask, generating intensity values for Ube3aYFP within each cell.

### Human Induced Pluripotent Stem Cell Derived Neurons

GABAergic and glutamatergic induced pluripotent stem cell (iPSC) derived neural precursor cells (NRC-100-010-001 and GNC-301-030-001, Cellular Dynamics International, Madison WI) were differentiated into neurons according to the manufactures protocol. Briefly, neural precursor cells were thawed and resuspended in chemically defined medium and added to sterile-culture plates coated with poly-D-lysine and laminin. The medium was replaced 24 hr after plating and then one-half of the medium was replaced every 3-5 days afterwards.

### RNA Isolation

For cultured iPSC-derived neurons, RNA isolation and cDNA synthesis were performed using the Cell-to-CT kit (Thermo Fisher Scientific) in a lysate volume of 55 μl.

### Analysis of RNA Levels

The steady state RNA levels of target transcripts were measured using TaqMan quantitative reverse-transcription PCR (qRT-PCR) assays. Total reaction volume was 10 uL, including 2 μl of cDNA, 1×Gene Expression Master mix (4369016, Thermo Fisher Scientific, Waltham, MA), and 1×TaqMan primer assay (Thermo Fisher Scientific). Cycling conditions were 2 minutes at 50° C., 10 minutes at 95° C., and 40 cycles of 15 seconds at 95° C. and 1 minute at 60° C., with readings taken at the 60° C. step of every cycle. Reactions were run on a BIO-RAD T1000 CFX96 thermocycler (Bio-Rad Laboratories, Hercules CA), with internal control (PPIA, Hs99999904_m1, Thermo Fisher Scientific) and target [UBE3A-AS, Hs01372957_m1; SNORD116-11, Hs04275268_gH; SNORD115, Hs04275288_gH; IPW, Hs03455409_s1; SNORD109A/B, AP47WVR (Thermo Fisher Scientific); UBE3A: forward ATATGTGGAAGCCGGAATCT (SEQ ID NO:500); reverse: CCCAGAACTCCCTAATCAGAA (SEQ ID NO:501); and, probe: ATGACGGTGGCTATACCAGG (SEQ ID NO:502)] reactions performed together. Data was retrieved and analyzed with the BIORAD CFX Maestro software (Bio-Rad Laboratories). Samples with internal control Cq values ≥30 were filtered. Quality of data was visually inspected to identify discrepancies between technical and/or plate replicates. Measurements for inferential statistics and descriptive statistics consist of ΔΔCq values (2^{-ΔΔCq}=2^{-(Cq[target]-Cq[internal control])-(Cq[target]-Cq[internal control])}).

### Example 2

### Identification of ASO Target Region

Analysis of RNA-sequencing data generated from mouse tissues and cells revealed a region located between the 3′-end of the Snord115 cluster and 5′-end of the Ube3a antisense (Ube3a-AS) transcript containing genetic elements believed to be important for processing of the Snord115 host-gene transcript and transcription of Ube3a-AS (FIGS. 6A-6D). Analysis of RNA-sequencing data generated from human tissues revealed a region located between the 3′-end of the SNORD115 cluster and SNORD109B (FIGS. 7A-7G) that contained elements similar to those observed in mouse; however, comparative analysis of this region indicated that there was little to no sequence conservation between human and rodents.

### Materials and Methods

### RNA-sequencing

RNA was isolated using Qiagen RNAeasy Plus (74136, Qiagen, Hilden, Germany). RNA concentration was determined using Qubit Fluorometric Quantitation (Thermo Fisher Scientific) and RNA quality was assessed using a 4200 Agilent TapeStation (Agilent, Santa Clara, CA). RNA-sequencing libraries were generated using the Illumina TruSeq Stranded Total RNA kit (20020597, Illumina, Inc., San Diego, CA) according to the manufacturer's protocol. 75 base-pair paired-end sequencing was performed using a NextSeq 500 (Illumina, San Diego, CA) at the Texas A&M Institute for Genome Sciences and Society Genomics core. Raw sequencing reads were processed using CASAVA. The resulting FASTQ sequences were examined using FASTQC.

FASTQ sequences were aligned to the human reference assembly (hg19) using Hisat2 (version 2.1.0), with the following settings: --fr. Aligned SAM sequences were then converted to binary BAM sequences, indexed, and sorted using Samtools. BAM files from individual samples were merged and indexed using Samtools. Aligned sequences were filtered using the view command in Samtools to remove non-uniquely aligned reads (quality>1).

A transcript assembly was generated for merged samples using Stringtie (version 1.3.4.d), with the following options: (stranded) --rf -f 0-j 2. Single exon transcripts were excluded from the assembled transcripts using gffread (GFF utilities, Johns Hopkins University, Center for Computational Biology).

### Example 3: Identification of Lead ASOs

Eighteen ASOs targeting the ASO-4 and ASO-6.1 target sequences and consisting of different backbone designs and RNA modifications were designed to identify potential lead ASOs (Table 17). Normal iPSC derived-neurons (GABAergic) were treated with a 10-point ½ log dose response curve of each ASO to compare the IC₅₀ and Eₘₐₓ values. Neural precursor cells were differentiated into neurons for at 18 DIV and then treated with a 10-point ½ log dose response ASOs [1 nM, 3 nM, 10 nM, 30 nM, 100 nM, 300 nM, 1 μM, 3 μM, 10 μM, and 30 μM (n=2)]. At 24 DIV, the steady state RNA levels of UBE3A-AS were measured and dose response curves fitted as described above (FIG. 8A and Table 18). The dose response curves were significantly different (Parallelism test: F_{(51,606)}=7.86; p<0.0001; R²=0.90), thus relative potencies were not estimated. Hierarchical clustering of the fitted curves revealed 3 Clusters of ASOs, with Cluster 1 representing the 9 most potent ASOs (FIGS. 8B and 8C). Analysis of Cluster 1 indicated that the ASOs had similar curves (Parallelism test: F_{(24,299)}=1.01; p=0.5; R²=0.93), and that ASO-4.4.PS.L was at least 3-times as potent as the other ASOs (Table 19). Further analysis, however, indicated that ASO-4.4.PS.L, ASO-6.1.PS.M, and ASO-6.1.PO-1.M had equivalent IC₅₀ values, whereas the other ASOs were slightly less potent (Table 20). Based on the relative potencies and internal selection criteria, ASO-4.4.PS.L and ASO-6.1.PO-1.O were investigated further.

TABLE 18
IC₅₀ and Eₘₐₓ of Candidate ASOs

| ASO | IC₅₀ (M) | IC₅₀ 95% CI (M) | | Eₘₐₓ | Eₘₐₓ 95% CI | | 30 μM (Mean) | Cluster |
|---|---|---|---|---|---|---|---|---|
| ASO-4.4.PS.L | 2.66E−08 | 3.66E−09 | 1.93E−07 | 0.0 | −0.23 | 0.23 | 0.05 | 1 |
| ASO-6.1.PS.M | 1.47E−07 | 6.80E−08 | 3.19E−07 | −0.05 | −0.21 | 0.12 | 0.02 | 1 |
| ASO-6.1.PO-1.M | 1.66E−07 | 7.15E−08 | 3.84E−07 | −0.02 | −0.20 | 0.16 | 0.04 | 1 |
| ASO-4.4.PO-1.L | 2.26E−07 | 8.95E−08 | 5.71E−07 | 0.04 | −0.17 | 0.25 | 0.1 | 1 |
| ASO-4.0.PO-1.M | 2.78E−07 | 1.52E−07 | 5.08E−07 | 0.02 | −0.11 | 0.15 | 0.05 | 1 |
| ASO-4.0.PS.M | 3.00E−07 | 1.80E−07 | 5.00E−07 | 0.05 | −0.06 | 0.15 | 0.05 | 1 |
| ASO-6.1.PO-1.O | 3.15E−07 | 7.98E−08 | 1.24E−06 | −0.1 | −0.50 | 0.26 | 0.04 | 1 |
| ASO-6.1.PS.L | 3.62E−07 | 1.37E−07 | 9.57E−07 | −0.07 | −0.32 | 0.18 | 0.04 | 1 |
| ASO-6.1.PS.O | 5.32E−07 | 1.20E−07 | 2.36E−06 | −0.2 | −0.67 | 0.29 | 0.05 | 1 |
| ASO-6.1.PO-2.L | 7.34E−07 | 5.35E−08 | 1.01E−05 | 0.3 | −0.11 | 0.76 | 0.4 | 2 |
| ASO-4.0.PO-1.O | 7.66E−07 | 3.70E−07 | 1.59E−06 | 0.05 | −0.12 | 0.23 | 0.1 | 2 |
| ASO-4.0.PS.O | 1.27E−06 | 5.13E−07 | 3.13E−06 | 0.06 | −0.20 | 0.31 | 0.1 | 2 |
| ASO-6.1.PO-1.L | 1.89E−06 | 4.42E−07 | 8.06E−06 | 0.03 | −0.34 | 0.39 | 0.2 | 2 |
| ASO-4.0.PO-2.O | 1.30E−04 | 1.65E−17 | 1.03E+09 | −0.3 | −9.51 | 8.94 | 0.6 | 2 |
| ASO-6.1.PO-2.M | 2.69E−04 | 9.85E−16 | 7.37E+07 | −1.2 | −13.16 | 10.77 | 0.3 | 2 |
| ASO-4.4.PO-2.L | 3.27E+01 | 0 | Inf | −2.7 | −577 | 571 | 0.6 | 3 |
| ASO-4.0.PO-2.M | 1.14E+05 | 0 | Inf | −76 | −74,958. | 74,805 | 0.5 | 3 |
| ASO-6.1.PO-2.O | 1.93E+10 | 0 | Inf | −5569 | −85,963,650 | 85,952,510 | 0.3 | 3 |

Full model parameter estimates from 4-parameter logistic regression model (Hill).
IC50 and confidence intervals represent molar concentration.
Emax and 30 uM values represent normalized UBE3A-AS RNA levels relative to vehicle.
Abbreviations: Inf, infinity; 95% CI, 95% confidence intervals

TABLE 19
Relative potency of ASOs in Cluster 1

| ASO | IC₅₀ (M) | Relative Potency | Std Error |
|---|---|---|---|
| ASO-4.4.PS.L | 5.03E−08 | 1 | 0 |
| ASO-6.1.PS.M | 1.53E−07 | 0.3 | 0.08 |
| ASO-6.1.PO-1.M | 1.77E−07 | 0.3 | 0.07 |
| ASO-6.1.PO-1.O | 1.99E−07 | 0.3 | 0.06 |
| ASO-4.0.PS.M | 2.62E−07 | 0.2 | 0.05 |
| ASO-4.0.PO-1.M | 2.78E−07 | 0.2 | 0.04 |
| ASO-6.1.PS.L | 2.81E−07 | 0.2 | 0.04 |
| ASO-4.4.PO-1.L | 3.22E−07 | 0.2 | 0.04 |
| ASO-6.1.PS.O | 4.32E−07 | 0.1 | 0.03 |

Parallel model parameter estimates from 4 Parameter logistic regression model (Hill).
Abbreviations:
M, molar;
Std, standard

TABLE 20
Equivalence of ASOs in Cluster 1 Relative to ASO-4.4.PS.L

| | | | | | Limit |
|---|---|---|---|---|---|
| | | IC₅₀ | Lower and Upper | | Ex- |
| ASO | ASO | Ratio | Confidence Limits | | ceeded |
| ASO-4.4.PS.L | ASO-6.1.PO-1.M | 0.90 | 0.81 | 0.98 | Equiva- |
| | | | | | lent |
| | ASO-6.1.PS.M | 0.90 | 0.82 | 0.98 | Equiva- |
| | | | | | lent |
| | ASO-4.0.PO-1.M | 0.87 | 0.79 | 0.94 | Lower |
| | ASO-4.0.PS.M | 0.86 | 0.79 | 0.94 | Lower |
| | ASO-4.4.PO-1.L | 0.88 | 0.79 | 0.96 | Lower |
| | ASO-6.1.PO-1.O | 0.86 | 0.77 | 0.95 | Lower |
| | ASO-6.1.PS.L | 0.85 | 0.77 | 0.93 | Lower |
| | ASO-6.1.PS.O | 0.83 | 0.73 | 0.92 | Lower |

Two one-sided Tests

### Materials and Methods

Methods were similar to those described in Example 2 unless noted otherwise.

### Example 4: Pharmacodynamic Analysis of ASO-6.1-PO-1.O and ASO-4.4.PS.L in Angelman Syndrome iPSC Neurons

The potencies of ASO-6.1.PS.O and ASO-4.4.PS.L were then examined in iPSC derived-neurons from an Angelman syndrome patient with a maternal derived deletion of the 15q11-q13 region. Induced pluripotent stem cells were differentiated into neurons and then treated with a 10-point ½ log dose response curve of ASO-6.1.PO-1.O and ASO-4.4.PS.L [1 nM, 3 nM, 10 nM, 30 nM, 100 nM, 300 nM, 1 μM, 3 μM, 10 μM, and 30 μM (n=3)]. Six days following treatment, the steady state RNA levels of UBE3A-AS were measured and dose response curves were fitted as described above (FIG. 9A). The dose response curves were similar between ASOs (Parallelism test: F_{(3,132)}=1.07, p=0.4, R²=0.82), with ASO-4.4.PS.L (437 nM) being approximately 2.7-fold more potent than ASO-6.1.PO-1.O (1.22 uM). The IC₅₀ values were equivalent [ASO-6.1.PO-1.O/ASO-4.4.PS.L IC₅₀ ratio:=0.96 (Lower confidence limit=0.9; Upper confidence limit=1.0)]. The Eₘₐₓ values were similar (30 μM: ASO-4.4.PS.L=0.01±0.0007; ASO-6.1.PO-1.O=0.05 ±0.004) but not considered equivalent due to the confidence intervals [ASO-6.1.PO-1.O/ASO-4.4.PS.L Eₘₐₓ ratio:=−9.1 (Lower confidence limit=−224; Upper confidence limit=205)].

### Materials and Methods

Methods were similar to those described in Example 2 unless noted otherwise.

Angelman syndrome induced pluripotent stem cells derived neurons Angelman syndrome iPS cells (AG1-0 iPSCs) (ECN001, Kerafast, Boston, MA) were co-cultured on irradiated murine embryonic fibroblasts in human embryonic stem cell medium [DMEM/F12 (11330-057, Gibco Biosciences, Dublin, Ireland), 20% Knockout Serum Replacement (10828-028, Thermo Fisher Scientific), 1× Non-essential amino acids, 2 mM L-glutamine, 7 μl/mL 2-Mercaptoethanol, and 4 μg/mL basic Fibroblast Growth Factor]. For the first passage, AG1-0 cells were passaged according to the product manual for PluriSTEM Human ES/IPS Medium (SCM130, Millipore Sigma, Burlington, MA), which is feeder-free and utilizes Dispase II (SCM133, Millipore Sigma) to dissociate cells. Matrigel™ hESC-qualified Matrix (354277, Corning BD Biosciences, Corning, NY) was used as an extracellular matrix. At the second passage, the matrix was switched to vitronectin (CC130, Millipore Sigma). During subsequent passages, areas of differentiation were manually removed until differentiated cells represented approximately <5% of the colonies. After four subsequent passages, AG1-0 cells were differentiated using the Millipore ES/IPS Neurogenesis Kit (SCR603, SCM110, and SCM111) but lacking vitronectin as an extracellular matrix. The initial passage was performed with EZ-LiFT (SCM139, Millipore Sigma) to obtain high quality iPS cells. Neural progenitor cells were frozen at stage zero (P₀) and subsequently thawed for differentiation. Differentiation was performed on sterile culture plates coated with poly-D-lysine (10 μg/mL) and laminin [10 μg/mL (23017-015, Gibco) in differentiation medium (SCM111) for 10 days of differentiation. In some instances, cells were differentiated in Cellular Dynamics Maintenance Medium (NRM-100-121-001, Cellular Dynamics International, Madison, WI).

### Example 5: Expression Analysis of the PWS Polycistronic Transcript in Angelman Syndrome iPSC Neurons Treated With ASO-6.1-PO-1.O and ASO-4.4.PS.L

To determine whether ASO-4.4.PS.L and ASO-6.1.PO-1.O affect the levels of RNA transcripts encoded by the PWS polycistronic transcript, RNA-sequencing was performed on AS iPS cells treated with each ASO and the steady state RNA levels of SNURF, SNRPN, the SNORD116 host-gene transcript (SNHG116), the SNORD116 snoRNAs, IPW, the SNORD115 host-gene transcript (SNHG115), the SNORD115 snoRNAs, and UBE3A-AS were quantified. UBE3A steady state RNA levels were also measured. Angelman syndrome iPS cells were differentiated into neurons as described above and then treated with vehicle (1% H2O, n=3), ASO-4.4.PS.L (30 u μM, n=3) and ASO-6.1.PO-1.O (30 μM, n=3). Six days post-treatment, RNA RNA-sequencing was performed on total RNA (rRNA depleted) isolated from the cultures. To generate annotations of the SNHG116, SNHG115, and UBE3A-AS transcripts, a transcriptome was assembled from the vehicle RNA-seq data and then incorporated into the reference gene annotation. Relative to vehicle, the steady state RNA levels of SNURF, SNRPN, SNHG116, the SNORD116 snoRNAs, and the SNORD115 snoRNAs were similar and not significantly different. ASO-4.4.PS.L, but not ASO-6.1.PO-1.O, reduced IPW levels (1.5-fold), but the effect was not significant. ASO-6.1.PO-1.O and ASO-4.4.PS.L significantly reduced SNHG115 and UBE3A-AS RNA levels. ASO-6.1.PO-1.O and ASO-4.4.PS.L had a similar effect on SNHG115 levels; however, ASO-4.4.PS.L had a much larger effect on UBE3A-AS RNA levels than ASO-6.1.PO-1.O (ASO-4.4.PS.L:-6.1-fold change; ASO-6.1.PO-1.O:-2.8-fold change). ASO treatment increased UBE3A RNA levels by approximately 1.2-fold, but the effect was not significant (FIG. 10 and Table 21).

TABLE 21
Effect of ASO Treatment on RNA Levels of
PWS Polycistronic Transcripts and UBE3A

| | | | Std | t | Adjusted |
|---|---|---|---|---|---|
| Gene | Treatment | Difference | Error | Ratio | P |
| SNURF | ASO-6.1.PO-1.O | −0.53 | 0.51 | −1.02 | 0.5 |
| | ASO-4.4.PS.L | 0.49 | 0.51 | 0.96 | 0.6 |
| SNRPN | ASO-6.1.PO-1.O | 0.03 | 0.11 | 0.30 | 0.9 |
| | ASO-4.4.PS.L | −0.02 | 0.11 | −0.16 | 1.0 |
| SNHG116 | ASO-6.1.PO-1.O | −0.07 | 0.10 | −0.75 | 0.7 |
| | ASO-4.4.PS.L | −0.24 | 0.10 | −2.49 | 0.08 |
| SNORD116 | ASO-6.1.PO-1.O | −0.04 | 0.46 | −0.08 | 1.0 |
| | ASO-4.4.PS.L | 0.27 | 0.45 | 0.60 | 0.8 |
| IPW | ASO-6.1.PO-1.O | 0.18 | 0.37 | 0.49 | 0.8 |
| | ASO-4.4.PS.L | −0.49 | 0.37 | −1.33 | 0.4 |
| SNH115G | ASO-6.1.PO-1.O | −0.55 | 0.09 | −5.92 | 0.002 |
| | ASO-4.4.PS.L | −0.58 | 0.09 | −6.33 | 0.001 |
| SNORD115 | ASO-6.1.PO-1.O | 0.24 | 0.52 | 0.45 | 0.8 |
| | ASO-4.4.PS.L | −0.26 | 0.49 | −0.54 | 0.8 |
| UBE3A-AS | ASO-6.1.PO-1.O | −1.48 | 0.06 | −24.17 | <0.0001 |
| | ASO-4.4.PS.L | −1.94 | 0.06 | −31.56 | <0.0001 |
| UBE3A | ASO-6.1.PO-1.O | 0.74 | 0.48 | 1.53 | 0.3 |
| | ASO-4.4.PS.L | 0.90 | 0.48 | 1.88 | 0.2 |

One way ANOVA with Dunnett's multiple comparison test relative to vehicle.

### Materials and Methods

Methods were similar to those described in Example 4 unless noted otherwise.

### Differential Expression Analysis of PWS RNAS

Normalized FPKM (fragments per thousand per million) values of the RefSeq gene annotation will be estimated using Cuffnorm with the default settings and the following option: -u. The FPKM values of each gene annotation was determined for each sample from the output file and used for descriptive and inferential statistics.

### Example 6: Pharmacodynamic Analysis of ASO-6.1-PO-1.O and ASO-4.4.PS.L in Cynomolgus Macaque

The ASO-4 and ASO-6 target regions are conserved across several non-human primate (NHP) species, thus enabling both safety and efficacy studies in a large animal model. To examine the efficacy of ASO-4.4.PS.L and ASO-6.1.PO-1.O in the central nervous system (CNS), ASOs were delivered to Cynomolgus macaques by intrathecal lumbar puncture. Animals were administered a single bolus injection of vehicle (0.9% saline, n=5), ASO-6.1.PO-1.O (10 mg, n=3), and ASO.4.4.PS.L (10 mg, n=3). Twenty-eight days following treatment, central nervous (CNS) tissues were collected and the steady state RNA levels of UBE3A-AS were measured. Overall, ASO-4.4.PS.L had a larger effect on UBE3A-AS RNA levels than ASO-6.1.PO-1.O (Table 22). ASO-4.4.PS.L reduced UBE3A-AS RNA in most CNS regions, with large effects in temporal lobe, primary motor cortex, pons, medulla, hippocampus, globus pallidus, frontal cortex (corona radiata), prefrontal cortex, and lumbar spinal cord. Similarly, ASO-6.1.PO-1.O reduced UBE3A-AS RNA levels in most CNS regions, with large effects observed in pons, oculomotor nucleus, and lumbar spinal cord (FIG. 11 and Table 23).

TABLE 22
Effect Size of ASO Treatment on UBE3A-AS RNA Levels in CNS

| Treatment | Treatment* | Cohen's d | 95% Confidence Intervals | | FDR |
|---|---|---|---|---|---|
| Vehicle | ASO-4.4.PS.L | 1.4 | 1.0 | 1.8 | 2.3E−10 |
| ASO-6.1.PO-1.O | ASO-4.4.PS.L | 1.0 | 0.6 | 1.5 | 6.4E−06 |
| Vehicle | ASO-6.1.PO-1.O | 0.3 | −0.06 | 0.7 | 0.09 |

Students t-test with FDR adjusted P values
Cohen's d effect sizes: 0.2, small; 0.5, medium; 0.8, large; 1.2, very large
Abbreviations: FDR, false discovery rate

TABLE 23
Effect of ASO Treatment on UBE3A-AS
RNA Levels in CNS Regions

| | | Differ- | Std | t | Adjusted |
|---|---|---|---|---|---|
| CNS Region | ASO | ence | Error | Ratio | P |
| Caudate Nucleus | ASO-6.1.PO-1.O | 0.10 | 0.22 | 0.46 | 0.9 |
| | ASO-4.4.PS.L | −0.21 | 0.22 | −0.94 | 0.6 |
| Cerebellum | ASO-6.1.PO-1.O | −0.11 | 0.09 | −1.15 | 0.5 |
| | ASO-4.4.PS.L | −0.05 | 0.09 | −0.53 | 0.8 |
| Frontal cortex | ASO-6.1.PO-1.O | 0.01 | 0.27 | 0.04 | 0.9 |
| | ASO-4.4.PS.L | −0.71 | 0.27 | −2.66 | 0.05 |
| Frontal Cortex | ASO-6.1.PO-1.O | −0.08 | 0.22 | −0.34 | 0.9 |
| (Corona radiata) | ASO-4.4.PS.L | −0.62 | 0.22 | −2.79 | 0.04 |
| Globus Pallidus | ASO-6.1.PO-1.O | 0.10 | 0.24 | 0.40 | 0.9 |
| | ASO-4.4.PS.L | −0.38 | 0.24 | −1.54 | 0.3 |
| Hippocampus | ASO-6.1.PO-1.O | −0.19 | 0.21 | −0.91 | 0.6 |
| | ASO-4.4.PS.L | −0.57 | 0.21 | −2.66 | 0.05 |
| Spinal Cord | ASO-6.1.PO-1.O | −0.32 | 0.20 | −1.63 | 0.2 |
| (Lumbar) | ASO-4.4.PS.L | −0.87 | 0.20 | −4.46 | 0.004 |
| Medulla | ASO-6.1.PO-1.O | −0.24 | 0.20 | −1.16 | 0.45 |
| | ASO-4.4.PS.L | −0.32 | 0.20 | −1.59 | 0.3 |
| Oculomotor | ASO-6.1.PO-1.O | −0.37 | 0.29 | −1.27 | 0.4 |
| Nucleus | ASO-4.4.PS.L | −0.18 | 0.29 | −0.62 | 0.8 |
| Pons | ASO-6.1.PO-1.O | −0.27 | 0.21 | −1.30 | 0.4 |
| | ASO-4.4.PS.L | −0.47 | 0.21 | −2.25 | 0.1 |
| Motor Cortex | ASO-6.1.PO-1.O | −0.19 | 0.30 | −0.65 | 0.8 |
| | ASO-4.4.PS.L | −0.59 | 0.30 | −1.99 | 0.1 |
| Putamen | ASO-6.1.PO-1.O | 0.07 | 0.15 | 0.44 | 0.9 |
| | ASO-4.4.PS.L | −0.04 | 0.15 | −0.25 | 0.9 |
| Temporal Lobe | ASO-6.1.PO-1.O | 0.13 | 0.25 | 0.54 | 0.8 |
| | ASO-4.4.PS.L | −0.59 | 0.25 | −2.39 | 0.08 |
| Thalamus | ASO-6.1.PO-1.O | −0.02 | 0.14 | −0.14 | 0.9 |
| | ASO-4.4.PS.L | −0.20 | 0.14 | −1.46 | 0.3 |

One way ANOVA with Dunnett's multiple comparison test relative to vehicle.

### Materials and Methods

### Administration of ASOs

NHP studies were performed at Northern Biomedical Research and Charles River Laboratories using protocols approved by the institutions respective Institutional Animal Care and Use Committees. Male and female Cynomolgus macaques (Macaca fascicularis) weighing 2-4 kg were anesthetized and single 1 mL dose of ASO or vehicle was administered via intrathecal lumbar puncture. The dosing solution was prepared by dissolution of lyophilized ASO in the vehicle control article (0.9% sodium chloride) and was filtered through a 0.2-μm filter. CNS and spinal cord samples were harvested, and the CNS was sectioned into 4-mm coronal slices. Tissue samples were flash frozen and stored at −80° C. until RNA isolation.

RNA isolation

A 4 mm tissue punch was taken from each region of interest of which approximately half was used for RNA isolation. RNA isolation was performed using the Qiagen RNeasy Plus Mini kit (74136, Qiagen) with tissue disruption and lysis performed with 5 mm stainless steel beads in a TissueLyser II. The RNA was eluted in two volumes of 30 μl water, for a total elution volume of 60 μl. RNA was quantified using the Qubit with the RNA XR assay (Q33224, Thermo Fisher Scientific). cDNA was synthesized from 2 μg of input RNA using the High Capacity RNA-to-cDNA kit (4387406, Thermo Fisher Scientific) in a total reaction volume of 50 μl.

### Analysis of UBE3A-AS RNA Levels in Tissues

Cynomolgus macaque UBE3A-AS RNA levels were estimated using SYBR Green quantitative reverse-transcription PCR (qRT-PCR). Total reaction volume was 10 μl, including 2 μl of cDNA, 1× PowerUp SYBR Green Master mix (A25741, Thermo Fisher Scientific), and 500 nM of each primer (forward and reverse). Cycling conditions were 2 minutes at 50° C., 2 minutes at 95° C., and 40 cycles of 15 seconds at 95° C. and 1 minute at 60° C., with readings taken at the 60° C. step of every cycle. Reactions were run on a BIO-RAD T1000 CFX96 thermocycler, with internal control (PPIA, forward: GTCTCCTTCGAGCTGTTTGC (SEQ ID NO:503); reverse: CCTTTCTCTCCAGTGCTCAGA (SEQ ID NO:504)) and target (UBE3A-AS, forward: CCTGTGAACTTTCAACCAGGA (SEQ ID NO:505); reverse: GGATCAGACTCCAGGCCTTC (SEQ ID NO:506)) reactions performed separately. Data was retrieved and initial analysis was done with the BIORAD CFX Maestro software, with in depth statistical analyses performed with Excel and JMP.

### Example 7: ASOs Targeting Exonic Boundaries of Spliced UBE3A-AS Transcripts

In some embodiments, the target sequence is an exonic boundary involving UBE3A-AS exons 1-5 and SNORD109B exons 1-2. Target sequences consist of 38 nucleotides (19 nucleotides of each exon) centered on the exonic boundary of each exon (19 nucleotides representing the 5′ and 3′-ends of adjacent exons). There were 12 segments of sequences, with exonic boundaries involving segments 1-2, 2-3, 3-4, 5-6, 7-8, 9-10, and 11-12. The chromosomal coordinates are provided in Table 24. A single merged junction sequence was created that shows the spliced exons (|, exonic junction) and intervening exonic sequences ([]). ASOs (20-, 19, and 18-mer) targeting the exonic junctions are provided in Table 25.

### Merged Junction-Sequence

AATGAAATCTTCTGATTTG|TAAGACATGCTGCCAAGAG[]ATTAGTTTTACACCTT CAG|GATAAAGACTGCTGAGAAG[]GTTTAAGGATGCTATTCTG|AAAAGACTGTG GAGGAAGA []TTAAGGAAACCATCTCTGG|GATAAGGATGACTGAGGAA[]ATTTAA GGATGCCACTCTG|GTTAAAAGCTGAAACAACT[]GAAACTTCAGGGAAAAGAG|A AGGCCTGGAATCTGATCC (SEQ ID NO:489).
   |=3′-5′ exonic junction
[]=intervening exonic sequence

TABLE 24
Chromosome 15 coordinates of targeted exonic junctions

| Segment | Start | End | Exonic Region |
|---|---|---|---|
| 1 | 25,511,743 | 25,511,761 | 3′ |
| 2 | 25,512,059 | 25,512,079 | 5′ |
| 3 | 25,512,175 | 25,512,191 | 3′ |
| 4 | 25,513,475 | 25,513,493 | 5′ |
| 5 | 25,513,582 | 25,513,600 | 3′ |
| 6 | 25,514,752 | 25,514,770 | 5′ |
| 7 | 25,514,863 | 25,514,881 | 3′ |
| 8 | 25,516,564 | 25,516,582 | 5′ |
| 9 | 25,516,663 | 25,516,681 | 3′ |
| 10 | 25,522,514 | 25,522,532 | 5′ |
| 11 | 25,522,537 | 25,522,556 | 3′ |
| 12 | 25,523,994 | 25,524,012 | 5′ |

Human chromosome 15 coordinates (hg19 reference assembly)

TABLE 25
List of Junction ASOs and corresponding target regions

| ASO size | Target Sequence (5′-3′) | | ASO sequence (5′-3′) | |
|---|---|---|---|---|
| 20-mer | GAAACCAUCUCUGGGAUAAG | SEQ ID NO: 393 | CTTATCCCAGAGATGGTTTC | SEQ ID NO: 441 |
| | AAACCAUCUCUGGGAUAAGG | SEQ ID NO: 394 | CCTTATCCCAGAGATGGTTT | SEQ ID NO: 442 |
| | AACCAUCUCUGGGAUAAGGA | SEQ ID NO: 395 | TCCTTATCCCAGAGATGGTT | SEQ ID NO: 443 |
| | ACCAUCUCUGGGAUAAGGAU | SEQ ID NO: 396 | ATCCTTATCCCAGAGATGGT | SEQ ID NO: 444 |
| | CCAUCUCUGGGAUAAGGAUG | SEQ ID NO: 397 | CATCCTTATCCCAGAGATGG | SEQ ID NO: 445 |
| | CAUCUCUGGGAUAAGGAUGA | SEQ ID NO: 398 | TCATCCTTATCCCAGAGATG | SEQ ID NO: 446 |
| | AUCUCUGGGAUAAGGAUGAC | SEQ ID NO: 399 | GTCATCCTTATCCCAGAGAT | SEQ ID NO: 447 |
| | UCUCUGGGAUAAGGAUGACU | SEQ ID NO: 400 | AGTCATCCTTATOCCAGAGA | SEQ ID NO: 448 |
| | CUCUGGGAUAAGGAUGACUG | SEQ ID NO: 401 | CAGTCATCCTTATCCCAGAG | SEQ ID NO: 449 |
| | UCUGGGAUAAGGAUGACUGA | SEQ ID NO: 402 | TCAGTCATCCTTATCCCAGA | SEQ ID NO: 450 |
| | CUGGGAUAAGGAUGACUGAG | SEQ ID NO: 403 | CTCAGTCATCCTTATCCCAG | SEQ ID NO: 451 |
| | UGGGAUAAGGAUGACUGAGG | SEQ ID NO: 404 | CCTCAGTCATCCTTATCCCA | SEQ ID NO: 452 |
| | GGGAUAAGGAUGACUGAGGA | SEQ ID NO: 405 | TCCTCAGTCATCCTTATCCC | SEQ ID NO: 453 |
| | GGAUAAGGAUGACUGAGGAA | SEQ ID NO: 406 | TTCCTCAGTCATCCTTATCC | SEQ ID NO: 454 |
| | GCUGAAACAACUGAAACUUC | SEQ ID NO: 407 | GAAGTTTCAGTTGTTTCAGC | SEQ ID NO: 455 |
| | GAAACAACUGAAACUUCAGG | SEQ ID NO: 408 | CCTGAAGTTTCAGTTGTTTC | SEQ ID NO: 456 |
| | AAACAACUGAAACUUCAGGG | SEQ ID NO: 409 | CCCTGAAGTTTCAGTTGTTT | SEQ ID NO: 457 |
| | AACAACUGAAACUUCAGGGA | SEQ ID NO: 410 | TCCCTGAAGTTTCAGTTGTT | SEQ ID NO: 458 |
| | ACAACUGAAACUUCAGGGAA | SEQ ID NO: 411 | TTCCCTGAAGTTTCAGTTGT | SEQ ID NO: 459 |
| | CAACUGAAACUUCAGGGAAA | SEQ ID NO: 412 | TTTCCCTGAAGTTTCAGTTG | SEQ ID NO: 460 |
| | ACUGAAACUUCAGGGAAAAG | SEQ ID NO: 413 | CTTTTCCCTGAAGTTTCAGT | SEQ ID NO: 461 |
| 19-mer | AACCAUCUCUGGGAUAAGG | SEQ ID NO: 414 | CCTTATCCCAGAGATGGTT | SEQ ID NO: 462 |
| | ACCAUCUCUGGGAUAAGGA | SEQ ID NO: 415 | TCCTTATCCCAGAGATGGT | SEQ ID NO: 463 |
| | CCAUCUCUGGGAUAAGGAU | SEQ ID NO: 416 | ATCCTTATCCCAGAGATGG | SEQ ID NO: 464 |
| | CAUCUCUGGGAUAAGGAUG | SEQ ID NO: 417 | CATCCTTATCCCAGAGATG | SEQ ID NO: 465 |
| | AUCUCUGGGAUAAGGAUGA | SEQ ID NO: 418 | TCATCCTTATCCCAGAGAT | SEQ ID NO: 466 |
| | UCUCUGGGAUAAGGAUGAC | SEQ ID NO: 419 | GTCATCCTTATOCCAGAGA | SEQ ID NO: 467 |
| | CUCUGGGAUAAGGAUGACU | SEQ ID NO: 420 | AGTCATCCTTATCCCAGAG | SEQ ID NO: 468 |
| | UCUGGGAUAAGGAUGACUG | SEQ ID NO: 421 | CAGTCATCCTTATCCCAGA | SEQ ID NO: 469 |
| | CUGGGAUAAGGAUGACUGA | SEQ ID NO: 422 | TCAGTCATCCTTATCCCAG | SEQ ID NO: 470 |
| | UGGGAUAAGGAUGACUGAG | SEQ ID NO: 423 | CTCAGTCATCCTTATCCCA | SEQ ID NO: 471 |
| | GGGAUAAGGAUGACUGAGG | SEQ ID NO: 424 | CCTCAGTCATCCTTATCCC | SEQ ID NO: 472 |
| | GGAUAAGGAUGACUGAGGA | SEQ ID NO: 425 | TCCTCAGTCATCCTTATCC | SEQ ID NO: 473 |
| | AACAACUGAAACUUCAGGG | SEQ ID NO: 426 | CCCTGAAGTTTCAGTTGTT | SEQ ID NO: 474 |
| | ACAACUGAAACUUCAGGGA | SEQ ID NO: 427 | TCCCTGAAGTTTCAGTIGT | SEQ ID NO: 475 |
| | CAACUGAAACUUCAGGGAA | SEQ ID NO: 428 | TTCCCTGAAGTTTCAGTTG | SEQ ID NO: 476 |
| | CAACUGAAACUUCAGGGAA | SEQ ID NO: 429 | TTCCCTGAAGTTTCAGTTG | SEQ ID NO: 477 |
| 18-mer | CCAUCUCUGGGAUAAGGA | SEQ ID NO: 430 | TCCTTATCCCAGAGATGG | SEQ ID NO: 478 |
| | CAUCUCUGGGAUAAGGAU | SEQ ID NO: 431 | ATCCTTATCCCAGAGATG | SEQ ID NO: 479 |
| | AUCUCUGGGAUAAGGAUG | SEQ ID NO: 432 | CATCCTTATCCCAGAGAT | SEQ ID NO: 480 |
| | UCUCUGGGAUAAGGAUGA | SEQ ID NO: 433 | TCATCCTTATCCCAGAGA | SEQ ID NO: 481 |
| | CUCUGGGAUAAGGAUGAC | SEQ ID NO: 434 | GTCATCCTTATCCCAGAG | SEQ ID NO: 482 |
| | UCUGGGAUAAGGAUGACU | SEQ ID NO: 435 | AGTCATCCTTATCCCAGA | SEQ ID NO: 483 |
| | CUGGGAUAAGGAUGACUG | SEQ ID NO: 436 | CAGTCATCCTTATOCCAG | SEQ ID NO: 484 |
| | UGGGAUAAGGAUGACUGA | SEQ ID NO: 437 | TCAGTCATCCTTATCCCA | SEQ ID NO: 485 |
| | GGGAUAAGGAUGACUGAG | SEQ ID NO: 438 | CTCAGTCATCCTTATCCC | SEQ ID NO: 486 |
| | GGAUAAGGAUGACUGAGG | SEQ ID NO: 439 | CCTCAGTCATCCTTATCC | SEQ ID NO: 487 |
| | ACAACUGAAACUUCAGGG | SEQ ID NO: 440 | CCCTGAAGTTTCAGTTGT | SEQ ID NO: 488 |

### Example 8: siRNA, shRNA, and CRISPR Guide RNAs Targeting UBE3a-AS Exons 1-5

As noted above, in some embodiments, the disclosed oligonucleotide is a functional nucleic acid, such as a siRNA, shRNA, or nuclease gRNA, that inhibits, mutates, or deletes the target nucleic acid sequence.

Examples of siRNA targeting UBE3a-AS exons 1-5 are provided in Table 26. Examples of shRNA targeting UBE3a-AS exons 1-5 are provided in Table 27. Examples of gRNA targeting UBE3a-AS exons 1-5 are provided in Table 28.

TABLE 26
siRNA targeting UBE3a-AS exons 1-5

| Target sequence | | siRNA | |
|---|---|---|---|
| CCCAGGUGUCCUUUAAUGAA | SEQ ID NO: 507 | TTCATTAAAGGACACCTGGG | SEQ ID NO: 538 |
| CCAGGUGUCCUUUAAUGAAA | SEQ ID NO: 508 | TTTCATTAAAGGACACCTGG | SEQ ID NO: 539 |
| UGAAAAUGCUCUUGACACCA | SEQ ID NO: 509 | TGGTGTCAAGAGCATTTTCA | SEQ ID NO: 540 |
| GAAAAUGCUCUUGACACCAA | SEQ ID NO: 510 | TTGGTGTCAAGAGCATTTTC | SEQ ID NO: 541 |
| AAAUGCUCUUGACACCAAUG | SEQ ID NO: 511 | CATTGGTGTCAAGAGCATTT | SEQ ID NO: 542 |
| AGAUCAGUAGCUUCCUUUAC | SEQ ID NO: 512 | GTAAAGGAAGCTACTGATCT | SEQ ID NO: 543 |
| UCAGUAGCUUCCUUUACCGA | SEQ ID NO: 513 | TCGGTAAAGGAAGCTACTGA | SEQ ID NO: 544 |
| UCUAGAACAUUGAGCUAUGG | SEQ ID NO: 514 | CCATAGCTCAATGTTCTAGA | SEQ ID NO: 545 |
| CUAGAACAUUGAGCUAUGGA | SEQ ID NO: 515 | TCCATAGCTCAATGTTCTAG | SEQ ID NO: 546 |
| AACAUUGAGCUAUGGAAGAC | SEQ ID NO: 516 | GTCTTCCATAGCTCAATGTT | SEQ ID NO: 547 |
| ACAUUGAGCUAUGGAAGACU | SEQ ID NO: 517 | AGTCTTCCATAGCTCAATGT | SEQ ID NO: 548 |
| CUAUGGAAGACUCCCACCUA | SEQ ID NO: 518 | TAGGTGGGAGTCTTCCATAG | SEQ ID NO: 549 |
| UAUGGAAGACUCCCACCUAA | SEQ ID NO: 519 | TTAGGTGGGAGTCTTCCATA | SEQ ID NO: 550 |
| CAAGUGCUACCGCACAGGCA | SEQ ID NO: 520 | TGCCTGTGCGGTAGCACTTG | SEQ ID NO: 551 |
| AAGUGCUACCGCACAGGCAU | SEQ ID NO: 521 | ATGCCTGTGCGGTAGCACTT | SEQ ID NO: 552 |
| UACCGCACAGGCAUGCUGCA | SEQ ID NO: 522 | TGCAGCATGCCTGTGCGGTA | SEQ ID NO: 553 |
| CAGGCAUGCUGCAGUGAAUU | SEQ ID NO: 523 | AATTCACTGCAGCATGCCTG | SEQ ID NO: 554 |
| AGGCAUGCUGCAGUGAAUUU | SEQ ID NO: 524 | AAATTCACTGCAGCATGCCT | SEQ ID NO: 555 |
| ACCGUUGUUUAAGGAUGCUA | SEQ ID NO: 525 | TAGCATCCTTAAACAACGGT | SEQ ID NO: 556 |
| CCGUUGUUUAAGGAUGCUAU | SEQ ID NO: 526 | ATAGCATCCTTAAACAACGG | SEQ ID NO: 557 |
| CUGUGGAGGAAGAAAACCCU | SEQ ID NO: 527 | AGGGTTTTCTTCCTCCACAG | SEQ ID NO: 558 |
| AAGAAAACCCUUUACCCUGU | SEQ ID NO: 528 | ACAGGGTAAAGGGTTTTCTT | SEQ ID NO: 559 |
| AGAAAACCCUUUACCCUGUU | SEQ ID NO: 529 | AACAGGGTAAAGGGTTTTCT | SEQ ID NO: 560 |
| CUCAACUGCCUGGCACUGAA | SEQ ID NO: 530 | TTCAGTGCCAGGCAGTTGAG | SEQ ID NO: 561 |
| AACUGCCUGGCACUGAAAAU | SEQ ID NO: 531 | ATTTTCAGTGCCAGGCAGTT | SEQ ID NO: 562 |
| ACUGCCUGGCACUGAAAAUG | SEQ ID NO: 532 | CATTTTCAGTGCCAGGCAGT | SEQ ID NO: 563 |
| GUGUUUAAGGAAACCAUCUC | SEQ ID NO: 533 | GAGATGGTTTCCTTAAACAC | SEQ ID NO: 564 |
| GUUUAAGGAAACCAUCUCUG | SEQ ID NO: 534 | CAGAGATGGTTTCCTTAAAC | SEQ ID NO: 565 |
| AGGAAACCAUCUCUGAUAAG | SEQ ID NO: 535 | CTTATCAGAGATGGTTTCCT | SEQ ID NO: 566 |
| UCUUUGGCUUGUUGACACCA | SEQ ID NO: 536 | TGGTGTCAACAAGCCAAAGA | SEQ ID NO: 567 |
| CUUUGGCUUGUUGACACCAG | SEQ ID NO: 537 | CTGGTGTCAACAAGCCAAAG | SEQ ID NO: 568 |

TABLE 27
shRNA targeting UBE3a-AS exons 1-5

| | |
|---|---|
| GGTGCCATTCTATTATAAAtaacctgacccattaTTTATAATAGAATGGCACCTTTTT | SEQ ID NO: 569 |
| GCTTTCATCAATAATGAAAtaacctgacccattaTTTCATTATTGATGAAAGCTTTTT | SEQ ID NO: 570 |
| GGTCTTTCATCAATAATGAtaacctgacccattaTCATTATTGATGAAAGACCTTTTT | SEQ ID NO: 571 |
| GAAATCTTCTGATTTGTAAtaacctgacccattaTTACAAATCAGAAGATTTCTTTTT | SEQ ID NO: 572 |
| GCACCTAAGGGAATTAGTAtaacctgacccattaTACTAATTCCCTTAGGTGCTTTTT | SEQ ID NO: 573 |
| GTTTCAACCAGGATTTAAAtaacctgacccattaTTTAAATCCTGGTTGAAACTTTTT | SEQ ID NO: 574 |
| GCTTTCAACCAGGATTTAAtaacctgacccattaTTAAATCCTGGTTGAAAGCTTTTT | SEQ ID NO: 575 |
| GGAGATGTGCCATTCTATAtaacctgacccattaTATAGAATGGCACATCTCCTTTTT | SEQ ID NO: 576 |
| GTCTTTCATCAATAATGAAtaacctgacccattaTTCATTATTGATGAAAGACTTTTT | SEQ ID NO: 577 |
| GATCAATAATGAAATCTTAtaacctgacccattaTAAGATTTCATTATTGATCTTTTT | SEQ ID NO: 578 |
| GTGTCTTTCATCAATAATAtaacctgacccattaTATTATTGATGAAAGACACTTTTT | SEQ ID NO: 579 |
| GCAATAATGAAATCTTCTAtaacctgacccattaTAGAAGATTTCATTATTGCTTTTT | SEQ ID NO: 580 |
| GCATGCTGCAGTGAATTTAtaacctgacccattaTAAATTCACTGCAGCATGCTTTTT | SEQ ID NO: 581 |
| GGAAATCTTCTGATTTGTAtaacctgacccattaTACAAATCAGAAGATTTCCTTTTT | SEQ ID NO: 582 |
| GGTATATTCTATCTAGAAAtaacctgacccattaTTTCTAGATAGAATATACCTTTTT | SEQ ID NO: 583 |
| GTGCTGCAGTGAATTTAAAtaacctgacccattaTTTAAATTCACTGCAGCACTTTTT | SEQ ID NO: 584 |
| GTGTGCCATTCTATTATAAtaacctgacccattaTTATAATAGAATGGCACACTTTTT | SEQ ID NO: 585 |
| GTTACCATCAGTGTTTAAAtaacctgacccattaTTTAAACACTGATGGTAACTTTTT | SEQ ID NO: 586 |
| GCCTGCAACCGTTGTTTAAtaacctgacccattaTTAAACAACGGTTGCAGGCTTTTT | SEQ ID NO: 587 |
| GTATGTCTTTCATCAATAAtaacctgacccattaTTATTGATGAAAGACATACTTTTT | SEQ ID NO: 588 |

TABLE 28
CRISPR Guide RNAs targeting UBE3a-AS exons 1-5

| Strand | Sequence | SEQ ID | PAM |
|---|---|---|---|
| − | ACACTGATGGTAAAGTGGAC | SEQ ID NO: 589 | TGG |
| − | TAGAATATACACGTCGGTAA | SEQ ID NO: 590 | AGG |
| − | TCAACTGTCCCAGTCACAAC | SEQ ID NO: 591 | AGG |
| − | TCTAGATAGAATATACACGT | SEQ ID NO: 592 | CGG |
| − | TCTAGATAGAATATACACGT | SEQ ID NO: 593 | CGG |
| − | CTCCCCATGCACACTTGAGA | SEQ ID NO: 594 | AGG |
| − | CATCCTTAAACAACGGTTGC | SEQ ID NO: 595 | AGG |
| − | GGTGTAAAACTAATTCCCTT | SEQ ID NO: 596 | AGG |
| − | AACAACGGTTGCAGGGACAG | SEQ ID NO: 597 | AGG |
| + | TATGGAAGACTCCCACCTAA | SEQ ID NO: 598 | GGG |
| + | CTATGGAAGACTCCCACCTA | SEQ ID NO: 599 | AGG |
| + | AAGCCTTCTCAAGTGTGCAT | SEQ ID NO: 600 | GGG |
| + | CTATCTAGAACATTGAGCTA | SEQ ID NO: 601 | TGG |
| + | ACCCTCTGGTGTTGTCACAG | SEQ ID NO: 602 | AGG |
| + | AACCCTTTACCCTGTTGTTC | SEQ ID NO: 603 | AGG |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A modified oligonucleotide comprising a contiguous nucleotide sequence of at least 18 ribonucleotides in length having nucleic acid complementarity to a contiguous portion of a nucleotide sequence selected from SEQ ID NO: 1, SEQ ID NO: 2, SEQ ID NO: 3, SEQ ID NO: 4, and SEQ ID NO: 5.

2. The modified oligonucleotide of claim 1, wherein the modified oligonucleotide is an siRNA, an shRNA, or a gRNA.

3. The modified oligonucleotide of claim 2, wherein the modified oligonucleotide is an siRNA.

4. The modified oligonucleotide of claim 3, wherein the siRNA comprises a nucleotide sequence selected from those provided Table 26.

5. The modified oligonucleotide of claim 3, wherein the modified oligonucleotide comprises a nucleotide sequence of SEQ ID NO: 543, 544, 545, 546, 547, 548, 549, or 550.

6. The modified oligonucleotide of claim 2, wherein the modified oligonucleotide is a shRNA.

7. The modified oligonucleotide of claim 6, wherein the shRNA comprises a nucleotide sequence selected from those provided in Table 27.

8. The modified oligonucleotide of claim 7, wherein the modified oligonucleotide comprises a nucleotide sequence of SEQ ID NO: 569, 576, 583, or 585.

9. The modified oligonucleotide of claim 2, wherein the modified oligonucleotide is a CRISPR guide RNA (gRNA).

10. The modified oligonucleotide of claim 9, wherein the gRNA comprises a nucleotide sequence selected from those provided in Table 28.

11. The modified oligonucleotide of claim 10, wherein the modified oligonucleotide comprises a nucleotide sequence of SEQ ID NO: 590, 592, 593, 596, 598, 599, or 601.

12. The modified oligonucleotide of claim 1, comprising one or more modified nucleosides.

13. The modified oligonucleotide of claim 12, wherein the one or more modified nucleosides is a 2′ sugar modified nucleoside.

14. The modified oligonucleotide of claim 13, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and lacked nucleic acid (LNA) nucleosides.

15. The modified oligonucleotide of claim 1, comprising one or more modified internucleoside linkage.

16. The modified oligonucleotide of claim 15, wherein the one or more modified internucleoside linkage is a phosphorothioate internucleoside linkage.

17. The modified oligonucleotide of claim 1, wherein the modified oligonucleotide is conjugated to one or more non-nucleotide moiety.

18. The modified oligonucleotide of claim 17, wherein the non-nucleotide moiety is an antibody or antibody fragment that targets the transferrin receptor.

19. A method of inhibiting, mutating, or deleting a UBE3A antisense transcript (UBE3A-AS) nucleic acid in a cell, the method comprising contacting the cell with the modified oligonucleotide of claim 1.

20. A method of increasing UBE3A expression in a cell, the method comprising contacting the cell with the modified oligonucleotide of claim 1.


SEQUENCE LISTING (605 sequences)

SEQ ID NO: 1 - DNA, 184 bp, synthetic construct
  misc_feature 1..184: note = Synthetic Construct
  source 1..184: mol_type = other DNA
atgatgatatggaagaaaagcactctttggcctgttgtgactgggacagttgacagcacccaggtgtcctttaatgaaaatgctcttgacaccaatgcatcctagcatcacagcttcaggaagccttctcaagtgtgcatggggagtactatgtctttcatcaataatgaaatcttctgatttg

SEQ ID NO: 2 - DNA, 133 bp, synthetic construct
  misc_feature 1..133: note = Synthetic Construct
  source 1..133: mol_type = other DNA
taagacatgctgccaagagatgtgccattctattataaaagatcagtagcttcctttaccgacgtgtatattctatctagaacattgagctatggaagactcccacctaagggaattagttttacaccttcag

SEQ ID NO: 3 - DNA, 125 bp, synthetic construct
  misc_feature 1..125: note = Synthetic Construct
  source 1..125: mol_type = other DNA
ataaagactgctgagaagagcaccctctggtgttgtcacagaggcaagtgctaccgcacaggcatgctgcagtgaatttaactgatcctctgtccctgcaaccgttgtttaaggatgctattctg

SEQ ID NO: 4 - DNA, 129 bp, synthetic construct
  misc_feature 1..129: note = Synthetic Construct
  source 1..129: mol_type = other DNA
aaaagactgtggaggaagaaaaccctttaccctgttgttcagggagaaactgacaccactcaactgcctggcactgaaaatgtggcatccagtccactttaccatcagtgtttaaggaaaccatctctg

SEQ ID NO: 5 - DNA, 117 bp, synthetic construct
  misc_feature 1..117: note = Synthetic Construct
  source 1..117: mol_type = other DNA
ataaggatgactgaggaagagtactctttggcttgttgacaccagcacagctgacacacccagatatctgtttggtctcctgtgaactttcaaccaggatttaaggatgccactctg

SEQ ID NO: 6 - DNA, 20 bp, synthetic construct
  misc_feature 1..20: note = Synthetic Construct
  source 1..20: mol_type = other DNA
tagaggtgaaggccaggcac

SEQ ID NO: 7 - DNA, 20 bp, synthetic construct
  misc_feature 1..20: note = Synthetic Construct
  source 1..20: mol_type = other DNA
gtactcttcctcagtcatcc

SEQ ID NO: 8 - DNA, 20 bp, synthetic construct
  misc_feature 1..20: note = Synthetic Construct
  source 1..20: mol_type = other DNA
tgtcagtttctccctgaaca

SEQ ID NO: 9 - DNA, 20 bp, synthetic construct
  misc_feature 1..20: note = Synthetic Construct
  source 1..20: mol_type = other DNA
tagaatggcacatctcttgg

SEQ ID NO: 10 - DNA, 20 bp, synthetic construct
  misc_feature 1..20: note = Synthetic Construct
  source 1..20: mol_type = other DNA
gttttcttcctccacagtct

SEQ ID NO: 11 - DNA, 20 bp, synthetic construct
  misc_feature 1..20: note = Synthetic Construct
  source 1..20: mol_type = other DNA
ctggtgtcaacaagccaaag

SEQ ID NO: 12 - RNA, 18 nt, synthetic construct
  misc_feature 1..18: note = Synthetic Construct
  source 1..18: mol_type = other RNA
gaaaatgctcttgacacc

SEQ ID NO: 13 - RNA, 19 nt, synthetic construct
  misc_feature 1..19: note = Synthetic Construct
  source 1..19: mol_type = other RNA
gaaaatgctcttgacacca

SEQ ID NO: 14 - RNA, 20 nt, synthetic construct
  misc_feature 1..20: note = Synthetic Construct
  source 1..20: mol_type = other RNA
gaaaatgctcttgacaccaa

SEQ ID NO: 15 - DNA, 18 bp, synthetic construct
  misc_feature 1..18: note = Synthetic Construct
  source 1..18: mol_type = other DNA
ggtgtcaagagcattttc

SEQ ID NO: 16 - DNA, 19 bp, synthetic construct
  misc_feature 1..19: note = Synthetic Construct
  source 1..19: mol_type = other DNA
tggtgtcaagagcattttc

SEQ ID NO: 17 - DNA, 20 bp, synthetic construct
  misc_feature 1..20: note = Synthetic Construct
  source 1..20: mol_type = other DNA
ttggtgtcaagagcattttc

SEQ ID NO: 18 - RNA, 18 nt, synthetic construct
  misc_feature 1..18: note = Synthetic Construct
  source 1..18: mol_type = other RNA
catgctgccaagagatgt

SEQ ID NO: 19 - RNA, 19 nt, synthetic construct
  misc_feature 1..19: note = Synthetic Construct
  source 1..19: mol_type = other RNA
catgctgccaagagatgtg

SEQ ID NO: 20 - RNA, 20 nt, synthetic construct
  misc_feature 1..20: note = Synthetic Construct
  source 1..20: mol_type = other RNA
catgctgccaagagatgtgc

SEQ ID NO: 21 - RNA, 18 nt, synthetic construct
  misc_feature 1..18: note = Synthetic Construct
  source 1..18: mol_type = other RNA
atgctgccaagagatgtg

SEQ ID NO: 22 - RNA, 19 nt, synthetic construct
  misc_feature 1..19: note = Synthetic Construct
  source 1..19: mol_type = other RNA
atgctgccaagagatgtgc

SEQ ID NO: 23 - RNA, 20 nt, synthetic construct
  misc_feature 1..20: note = Synthetic Construct
  source 1..20: mol_type = other RNA
atgctgccaagagatgtgcc

SEQ ID NO: 24 - RNA, 19 nt, synthetic construct
  misc_feature 1..19: note = Synthetic Construct
  source 1..19: mol_type = other RNA
tgctgccaagagatgtgcc

SEQ ID NO: 25 - RNA, 20 nt, synthetic construct
  misc_feature 1..20: note = Synthetic Construct
  source 1..20: mol_type = other RNA
tgctgccaagagatgtgcca

SEQ ID NO: 26 - RNA, 19 nt, synthetic construct
  misc_feature 1..19: note = Synthetic Construct
  source 1..19: mol_type = other RNA
gctgccaagagatgtgcca

SEQ ID NO: 27 - RNA, 20 nt, synthetic construct
  misc_feature 1..20: note = Synthetic Construct
  source 1..20: mol_type = other RNA
gctgccaagagatgtgccat

SEQ ID NO: 28 - RNA, 18 nt, synthetic construct
  misc_feature 1..18: note = Synthetic Construct
  source 1..18: mol_type = other RNA
ctgccaagagatgtgcca

SEQ ID NO: 29 - RNA, 19 nt, synthetic construct
  misc_feature 1..19: note = Synthetic Construct
  source 1..19: mol_type = other RNA
ctgccaagagatgtgccat

SEQ ID NO: 30 - RNA, 20 nt, synthetic construct
  misc_feature 1..20: note = Synthetic Construct
  source 1..20: mol_type = other RNA
ctgccaagagatgtgccatt

SEQ ID NO: 31 - RNA, 18 nt, synthetic construct
  misc_feature 1..18: note = Synthetic Construct
  source 1..18: mol_type = other RNA
tgccaagagatgtgccat

SEQ ID NO: 32 - RNA, 19 nt, synthetic construct
  misc_feature 1..19: note = Synthetic Construct
  source 1..19: mol_type = other RNA
tgccaagagatgtgccatt

SEQ ID NO: 33 - RNA, 20 nt, synthetic construct
  misc_feature 1..20: note = Synthetic Construct
  source 1..20: mol_type = other RNA
tgccaagagatgtgccattc

SEQ ID NO: 34 - RNA, 18 nt, synthetic construct
  misc_feature 1..18: note = Synthetic Construct
  source 1..18: mol_type = other RNA
gccaagagatgtgccatt

SEQ ID NO: 35 - RNA, 19 nt, synthetic construct
  misc_feature 1..19: note = Synthetic Construct
  source 1..19: mol_type = other RNA
gccaagagatgtgccattc

SEQ ID NO: 36 - RNA, 20 nt, synthetic construct
  misc_feature 1..20: note = Synthetic Construct
  source 1..20: mol_type = other RNA
gccaagagatgtgccattct

SEQ ID NO: 37 - RNA, 18 nt, synthetic construct
  misc_feature 1..18: note = Synthetic Construct
  source 1..18: mol_type = other RNA
ccaagagatgtgccattc

SEQ ID NO: 38 - RNA, 19 nt, synthetic construct
  misc_feature 1..19: note = Synthetic Construct
  source 1..19: mol_type = other RNA
ccaagagatgtgccattct

SEQ ID NO: 39 - RNA, 20 nt, synthetic construct
  misc_feature 1..20: note = Synthetic Construct
  source 1..20: mol_type = other RNA
ccaagagatgtgccattcta

SEQ ID NO: 40 - RNA, 18 nt, synthetic construct
  misc_feature 1..18: note = Synthetic Construct
  source 1..18: mol_type = other RNA
caagagatgtgccattct

SEQ ID NO: 41 - RNA, 19 nt, synthetic construct
  misc_feature 1..19: note = Synthetic Construct
  source 1..19: mol_type = other RNA
caagagatgtgccattcta

SEQ ID NO: 42 - RNA, 20 nt, synthetic construct
  misc_feature 1..20: note = Synthetic Construct
  source 1..20: mol_type = other RNA
caagagatgtgccattctat

SEQ ID NO: 43 - RNA, 18 nt, synthetic construct
  misc_feature 1..18: note = Synthetic Construct
  source 1..18: mol_type = other RNA
tcctttaccgacgtgtat

SEQ ID NO: 44 - RNA, 19 nt, synthetic construct
  misc_feature 1..19: note = Synthetic Construct
  source 1..19: mol_type = other RNA
tcctttaccgacgtgtata

SEQ ID NO: 45 - RNA, 20 nt, synthetic construct
  misc_feature 1..20: note = Synthetic Construct
  source 1..20: mol_type = other RNA
tcctttaccgacgtgtatat

SEQ ID NO: 46 - RNA, 18 nt, synthetic construct
  misc_feature 1..18: note = Synthetic Construct
  source 1..18: mol_type = other RNA
cctttaccgacgtgtata

SEQ ID NO: 47 - RNA, 19 nt, synthetic construct
  misc_feature 1..19: note = Synthetic Construct
  source 1..19: mol_type = other RNA
cctttaccgacgtgtatat

SEQ ID NO: 48 - RNA, 20 nt, synthetic construct
  misc_feature 1..20: note = Synthetic Construct
  source 1..20: mol_type = other RNA
cctttaccgacgtgtatatt

SEQ ID NO: 49 - RNA, 20 nt, synthetic construct
  misc_feature 1..20: note = Synthetic Construct
  source 1..20: mol_type = other RNA
accgacgtgtatattctatc

SEQ ID NO: 50 - RNA, 19 nt, synthetic construct
  misc_feature 1..19: note = Synthetic Construct
  source 1..19: mol_type = other RNA
ccgacgtgtatattctatc

SEQ ID NO: 51 - RNA, 20 nt, synthetic construct
  misc_feature 1..20: note = Synthetic Construct
  source 1..20: mol_type = other RNA
ccgacgtgtatattctatct

SEQ ID NO: 52 - RNA, 20 nt, synthetic construct
  misc_feature 1..20: note = Synthetic Construct
  source 1..20: mol_type = other RNA
tctagaacattgagctatgg

SEQ ID NO: 53 - RNA, 18 nt, synthetic construct
  misc_feature 1..18: note = Synthetic Construct
  source 1..18: mol_type = other RNA
cattgagctatggaagac

SEQ ID NO: 54 - RNA, 20 nt, synthetic construct
  misc_feature 1..20: note = Synthetic Construct
  source 1..20: mol_type = other RNA
ctatggaagactcccaccta

SEQ ID NO: 55 - RNA, 19 nt, synthetic construct
  misc_feature 1..19: note = Synthetic Construct
  source 1..19: mol_type = other RNA
tatggaagactcccaccta

SEQ ID NO: 56 - RNA, 20 nt, synthetic construct
  misc_feature 1..20: note = Synthetic Construct
  source 1..20: mol_type = other RNA
tatggaagactcccacctaa

SEQ ID NO: 57 - RNA, 18 nt, synthetic construct
  misc_feature 1..18: note = Synthetic Construct
  source 1..18: mol_type = other RNA
atggaagactcccaccta

SEQ ID NO: 58 - RNA, 19 nt, synthetic construct
  misc_feature 1..19: note = Synthetic Construct
  source 1..19: mol_type = other RNA
atggaagactcccacctaa

SEQ ID NO: 59 - RNA, 18 nt, synthetic construct
  misc_feature 1..18: note = Synthetic Construct
  source 1..18: mol_type = other RNA
tggaagactcccacctaa

SEQ ID NO: 60 - RNA, 20 nt, synthetic construct
  misc_feature 1..20: note = Synthetic Construct
  source 1..20: mol_type = other RNA
gactcccacctaagggaatt

SEQ ID NO: 61 - RNA, 18 nt, synthetic construct
  misc_feature 1..18: note = Synthetic Construct
  source 1..18: mol_type = other RNA
actcccacctaagggaat

SEQ ID NO: 62 - RNA, 19 nt, synthetic construct
  misc_feature 1..19: note = Synthetic Construct
  source 1..19: mol_type = other RNA
actcccacctaagggaatt

SEQ ID NO: 63 - RNA, 20 nt, synthetic construct
  misc_feature 1..20: note = Synthetic Construct
  source 1..20: mol_type = other RNA
actcccacctaagggaatta

SEQ ID NO: 64 - RNA, 18 nt, synthetic construct
  misc_feature 1..18: note = Synthetic Construct
  source 1..18: mol_type = other RNA
ctcccacctaagggaatt

SEQ ID NO: 65 - RNA, 19 nt, synthetic construct
  misc_feature 1..19: note = Synthetic Construct
  source 1..19: mol_type = other RNA
ctcccacctaagggaatta

SEQ ID NO: 66 - RNA, 18 nt, synthetic construct
  misc_feature 1..18: note = Synthetic Construct
  source 1..18: mol_type = other RNA
tcccacctaagggaatta

SEQ ID NO: 67 - DNA, 18 bp, synthetic construct
  misc_feature 1..18: note = Synthetic Construct
  source 1..18: mol_type = other DNA
acatctcttggcagcatg

SEQ ID NO: 68 - DNA, 19 bp, synthetic construct
  misc_feature 1..19: note = Synthetic Construct
  source 1..19: mol_type = other DNA
cacatctcttggcagcatg

SEQ ID NO: 69 - DNA, 20 bp, synthetic construct
  misc_feature 1..20: note = Synthetic Construct
  source 1..20: mol_type = other DNA
gcacatctcttggcagcatg

SEQ ID NO: 70 - DNA, 18 bp, synthetic construct
  misc_feature 1..18: note = Synthetic Construct
  source 1..18: mol_type = other DNA
cacatctcttggcagcat

SEQ ID NO: 71 - DNA, 19 bp, synthetic construct
  misc_feature 1..19: note = Synthetic Construct
  source 1..19: mol_type = other DNA
gcacatctcttggcagcat

SEQ ID NO: 72 - DNA, 20 bp, synthetic construct
  misc_feature 1..20: note = Synthetic Construct
  source 1..20: mol_type = other DNA
ggcacatctcttggcagcat

SEQ ID NO: 73 - DNA, 19 bp, synthetic construct
  misc_feature 1..19: note = Synthetic Construct
  source 1..19: mol_type = other DNA
ggcacatctcttggcagca

SEQ ID NO: 74 - DNA, 20 bp, synthetic construct
  misc_feature 1..20: note = Synthetic Construct
  source 1..20: mol_type = other DNA
tggcacatctcttggcagca

SEQ ID NO: 75 - DNA, 19 bp, synthetic construct
  misc_feature 1..19: note = Synthetic Construct
  source 1..19: mol_type = other DNA
tggcacatctcttggcagc

SEQ ID NO: 76 - DNA, 20 bp, synthetic construct
  misc_feature 1..20: note = Synthetic Construct
  source 1..20: mol_type = other DNA
atggcacatctcttggcagc

SEQ ID NO: 77 - DNA, 18 bp, synthetic construct
  misc_feature 1..18: note = Synthetic Construct
  source 1..18: mol_type = other DNA
tggcacatctcttggcag

SEQ ID NO: 78 - DNA, 19 bp, synthetic construct
  misc_feature 1..19: note = Synthetic Construct
  source 1..19: mol_type = other DNA
atggcacatctcttggcag

SEQ ID NO: 79 - DNA, 20 bp, synthetic construct
  misc_feature 1..20: note = Synthetic Construct
  source 1..20: mol_type = other DNA
aatggcacatctcttggcag

SEQ ID NO: 80 - DNA, 18 bp, synthetic construct
  misc_feature 1..18: note = Synthetic Construct
  source 1..18: mol_type = other DNA
atggcacatctcttggca

SEQ ID NO: 81 - DNA, 19 bp, synthetic construct
  misc_feature 1..19: note = Synthetic Construct
  source 1..19: mol_type = other DNA
aatggcacatctcttggca

SEQ ID NO: 82 - DNA, 20 bp, synthetic construct
  misc_feature 1..20: note = Synthetic Construct
  source 1..20: mol_type = other DNA
gaatggcacatctcttggca

SEQ ID NO: 83 - DNA, 18 bp, synthetic construct
  misc_feature 1..18: note = Synthetic Construct
  source 1..18: mol_type = other DNA
aatggcacatctcttggc

SEQ ID NO: 84 - DNA, 19 bp, synthetic construct
  misc_feature 1..19: note = Synthetic Construct
  source 1..19: mol_type = other DNA
gaatggcacatctcttggc

SEQ ID NO: 85 - DNA, 20 bp, synthetic construct
  misc_feature 1..20: note = Synthetic Construct
  source 1..20: mol_type = other DNA
agaatggcacatctcttggc

SEQ ID NO: 86 - DNA, 18 bp, synthetic construct
  misc_feature 1..18: note = Synthetic Construct
  source 1..18: mol_type = other DNA
gaatggcacatctcttgg

SEQ ID NO: 87 - DNA, 19 bp, synthetic construct
  misc_feature 1..19: note = Synthetic Construct
  source 1..19: mol_type = other DNA
agaatggcacatctcttgg

SEQ ID NO: 88 - DNA, 20 bp, synthetic construct
  misc_feature 1..20: note = Synthetic Construct
  source 1..20: mol_type = other DNA
tagaatggcacatctcttgg

SEQ ID NO: 89 - DNA, 18 bp, synthetic construct
  misc_feature 1..18: note = Synthetic Construct
  source 1..18: mol_type = other DNA
agaatggcacatctcttg

SEQ ID NO: 90 - DNA, 19 bp, synthetic construct
  misc_feature 1..19: note = Synthetic Construct
  source 1..19: mol_type = other DNA
tagaatggcacatctcttg

SEQ ID NO: 91 - DNA, 20 bp, synthetic construct
  misc_feature 1..20: note = Synthetic Construct
  source 1..20: mol_type = other DNA
atagaatggcacatctcttg

SEQ ID NO: 92 - DNA, 18 bp, synthetic construct
  misc_feature 1..18: note = Synthetic Construct
  source 1..18: mol_type = other DNA
atacacgtcggtaaagga

SEQ ID NO: 93 - DNA, 19 bp, synthetic construct
  misc_feature 1..19: note = Synthetic Construct
  source 1..19: mol_type = other DNA
tatacacgtcggtaaagga

SEQ ID NO: 94 - DNA, 20 bp, synthetic construct
  misc_feature 1..20: note = Synthetic Construct
  source 1..20: mol_type = other DNA
atatacacgtcggtaaagga

SEQ ID NO: 95 - DNA, 18 bp, synthetic construct
  misc_feature 1..18: note = Synthetic Construct
  source 1..18: mol_type = other DNA
tatacacgtcggtaaagg

SEQ ID NO: 96 - DNA, 19 bp, synthetic construct
  misc_feature 1..19: note = Synthetic Construct
  source 1..19: mol_type = other DNA
atatacacgtcggtaaagg

SEQ ID NO: 97 - DNA, 20 bp, synthetic construct
  misc_feature 1..20: note = Synthetic Construct
  source 1..20: mol_type = other DNA
aatatacacgtcggtaaagg

SEQ ID NO: 98 - DNA, 20 bp, synthetic construct
  misc_feature 1..20: note = Synthetic Construct
  source 1..20: mol_type = other DNA
gatagaatatacacgtcggt

SEQ ID NO: 99 - DNA, 19 bp, synthetic construct
  misc_feature 1..19: note = Synthetic Construct
  source 1..19: mol_type = other DNA
gatagaatatacacgtcgg

SEQ ID NO: 100 - DNA, 20 bp, synthetic construct
  misc_feature 1..20: note = Synthetic Construct
  source 1..20: mol_type = other DNA
agatagaatatacacgtcgg

SEQ ID NO: 101 - DNA, 20 bp, synthetic construct
  misc_feature 1..20: note = Synthetic Construct
  source 1..20: mol_type = other DNA
ccatagctcaatgttctaga

SEQ ID NO: 102 - DNA, 18 bp, synthetic construct
  misc_feature 1..18: note = Synthetic Construct
  source 1..18: mol_type = other DNA
gtcttccatagctcaatg

SEQ ID NO: 103 - DNA, 20 bp, synthetic construct
  misc_feature 1..20: note = Synthetic Construct
  source 1..20: mol_type = other DNA
taggtgggagtcttccatag

SEQ ID NO: 104 - DNA, 19 bp, synthetic construct
  misc_feature 1..19: note = Synthetic Construct
  source 1..19: mol_type = other DNA
taggtgggagtcttccata

SEQ ID NO: 105 - DNA, 20 bp, synthetic construct
  misc_feature 1..20: note = Synthetic Construct
  source 1..20: mol_type = other DNA
ttaggtgggagtcttccata

SEQ ID NO: 106 - DNA, 18 bp, synthetic construct
  misc_feature 1..18: note = Synthetic Construct
  source 1..18: mol_type = other DNA
taggtgggagtcttccat

SEQ ID NO: 107 - DNA, 19 bp, synthetic construct
  misc_feature 1..19: note = Synthetic Construct
  source 1..19: mol_type = other DNA
ttaggtgggagtcttccat

SEQ ID NO: 108 - DNA, 18 bp, synthetic construct
  misc_feature 1..18: note = Synthetic Construct
  source 1..18: mol_type = other DNA
ttaggtgggagtcttcca

SEQ ID NO: 109 - DNA, 20 bp, synthetic construct
  misc_feature 1..20: note = Synthetic Construct
  source 1..20: mol_type = other DNA
aattcccttaggtgggagtc

SEQ ID NO: 110 - DNA, 18 bp, synthetic construct
  misc_feature 1..18: note = Synthetic Construct
  source 1..18: mol_type = other DNA
attcccttaggtgggagt

SEQ ID NO: 111 - DNA, 19 bp, synthetic construct
  misc_feature 1..19: note = Synthetic Construct
  source 1..19: mol_type = other DNA
aattcccttaggtgggagt

SEQ ID NO: 112 - DNA, 20 bp, synthetic construct
  misc_feature 1..20: note = Synthetic Construct
  source 1..20: mol_type = other DNA
taattcccttaggtgggagt

SEQ ID NO: 113 - DNA, 18 bp, synthetic construct
  misc_feature 1..18: note = Synthetic Construct
  source 1..18: mol_type = other DNA
aattcccttaggtgggag

SEQ ID NO: 114 - DNA, 19 bp, synthetic construct
  misc_feature 1..19: note = Synthetic Construct
  source 1..19: mol_type = other DNA
taattcccttaggtgggag

SEQ ID NO: 115 - DNA, 18 bp, synthetic construct
  misc_feature 1..18: note = Synthetic Construct
  source 1..18: mol_type = other DNA
taattcccttaggtggga

SEQ ID NO: 116 - RNA, 20 nt, synthetic construct
  misc_feature 1..20: note = Synthetic Construct
  source 1..20: mol_type = other RNA
gataaagactgctgagaaga

SEQ ID NO: 117 - RNA, 20 nt, synthetic construct
  misc_feature 1..20: note = Synthetic Construct
  source 1..20: mol_type = other RNA
ataaagactgctgagaagag

SEQ ID NO: 118 - RNA, 20 nt, synthetic construct
  misc_feature 1..20: note = Synthetic Construct
  source 1..20: mol_type = other RNA
taaagactgctgagaagagc

SEQ ID NO: 119 - RNA, 20 nt, synthetic construct
  misc_feature 1..20: note = Synthetic Construct
  source 1..20: mol_type = other RNA
aaagactgctgagaagagca

SEQ ID NO: 120 - RNA, 20 nt, synthetic construct
  misc_feature 1..20: note = Synthetic Construct
  source 1..20: mol_type = other RNA
aagactgctgagaagagcac

SEQ ID NO: 121 - RNA, 20 nt, synthetic construct
  misc_feature 1..20: note = Synthetic Construct
  source 1..20: mol_type = other RNA
agactgctgagaagagcacc

SEQ ID NO: 122 - RNA, 20 nt, synthetic construct
  misc_feature 1..20: note = Synthetic Construct
  source 1..20: mol_type = other RNA
gactgctgagaagagcaccc

SEQ ID NO: 123 - RNA, 20 nt, synthetic construct
  misc_feature 1..20: note = Synthetic Construct
  source 1..20: mol_type = other RNA
caagtgctaccgcacaggca

SEQ ID NO: 124 - RNA, 20 nt, synthetic construct
  misc_feature 1..20: note = Synthetic Construct
  source 1..20: mol_type = other RNA
aagtgctaccgcacaggcat

SEQ ID NO: 125 - RNA, 20 nt, synthetic construct
  misc_feature 1..20: note = Synthetic Construct
  source 1..20: mol_type = other RNA
agtgctaccgcacaggcatg

SEQ ID NO: 126 - RNA, 20 nt, synthetic construct
  misc_feature 1..20: note = Synthetic Construct
  source 1..20: mol_type = other RNA
tgctaccgcacaggcatgct

SEQ ID NO: 127 - RNA, 20 nt, synthetic construct
  misc_feature 1..20: note = Synthetic Construct
  source 1..20: mol_type = other RNA
taccgcacaggcatgctgca

SEQ ID NO: 128 - RNA, 20 nt, synthetic construct
  misc_feature 1..20: note = Synthetic Construct
  source 1..20: mol_type = other RNA
gcacaggcatgctgcagtga

SEQ ID NO: 129 - RNA, 20 nt, synthetic construct
  misc_feature 1..20: note = Synthetic Construct
  source 1..20: mol_type = other RNA
cacaggcatgctgcagtgaa

SEQ ID NO: 130 - RNA, 20 nt, synthetic construct
  misc_feature 1..20: note = Synthetic Construct
  source 1..20: mol_type = other RNA
acaggcatgctgcagtgaat

SEQ ID NO: 131 - RNA, 20 nt, synthetic construct
  misc_feature 1..20: note = Synthetic Construct
  source 1..20: mol_type = other RNA
caggcatgctgcagtgaatt

SEQ ID NO: 132 - RNA, 20 nt, synthetic construct
  misc_feature 1..20: note = Synthetic Construct
  source 1..20: mol_type = other RNA
aggcatgctgcagtgaattt

SEQ ID NO: 133 - RNA, 20 nt, synthetic construct
  misc_feature 1..20: note = Synthetic Construct
  source 1..20: mol_type = other RNA
ggcatgctgcagtgaattta

SEQ ID NO: 134 - RNA, 20 nt, synthetic construct
  misc_feature 1..20: note = Synthetic Construct
  source 1..20: mol_type = other RNA
gcatgctgcagtgaatttaa

SEQ ID NO: 135 - RNA, 20 nt, synthetic construct
  misc_feature 1..20: note = Synthetic Construct
  source 1..20: mol_type = other RNA
catgctgcagtgaatttaac

SEQ ID NO: 136 - RNA, 20 nt, synthetic construct
  misc_feature 1..20: note = Synthetic Construct
  source 1..20: mol_type = other RNA
gcagtgaatttaactgatcc

SEQ ID NO: 137 - RNA, 20 nt, synthetic construct
  misc_feature 1..20: note = Synthetic Construct
  source 1..20: mol_type = other RNA
tccctgcaaccgttgtttaa

SEQ ID NO: 138 - RNA, 20 nt, synthetic construct
  misc_feature 1..20: note = Synthetic Construct
  source 1..20: mol_type = other RNA
ccctgcaaccgttgtttaag

SEQ ID NO: 139 - DNA, 20 bp, synthetic construct
  misc_feature 1..20: note = Synthetic Construct
  source 1..20: mol_type = other DNA
tcttctcagcagtctttatc

SEQ ID NO: 140 - DNA, 20 bp, synthetic construct
  misc_feature 1..20: note = Synthetic Construct
  source 1..20: mol_type = other DNA
ctcttctcagcagtctttat

SEQ ID NO: 141 - DNA, 20 bp, synthetic construct
  misc_feature 1..20: note = Synthetic Construct
  source 1..20: mol_type = other DNA
gctcttctcagcagtcttta

SEQ ID NO: 142 - DNA, 20 bp, synthetic construct
  misc_feature 1..20: note = Synthetic Construct
  source 1..20: mol_type = other DNA
tgctcttctcagcagtcttt

SEQ ID NO: 143 - DNA, 20 bp, synthetic construct
  misc_feature 1..20: note = Synthetic Construct
  source 1..20: mol_type = other DNA
gtgctcttctcagcagtctt

SEQ ID NO: 144 - DNA, 20 bp, synthetic construct
  misc_feature 1..20: note = Synthetic Construct
  source 1..20: mol_type = other DNA
ggtgctcttctcagcagtct

SEQ ID NO: 145 - DNA, 20 bp, synthetic construct
  misc_feature 1..20: note = Synthetic Construct
  source 1..20: mol_type = other DNA
gggtgctcttctcagcagtc

SEQ ID NO: 146 - DNA, 20 bp, synthetic construct
  misc_feature 1..20: note = Synthetic Construct
  source 1..20: mol_type = other DNA
tgcctgtgcggtagcacttg

SEQ ID NO: 147 - DNA, 20 bp, synthetic construct
  misc_feature 1..20: note = Synthetic Construct
  source 1..20: mol_type = other DNA
atgcctgtgcggtagcactt

SEQ ID NO: 148 - DNA, 20 bp, synthetic construct
  misc_feature 1..20: note = Synthetic Construct
  source 1..20: mol_type = other DNA
catgcctgtgcggtagcact

SEQ ID NO: 149 - DNA, 20 bp, synthetic construct
  misc_feature 1..20: note = Synthetic Construct
  source 1..20: mol_type = other DNA
agcatgcctgtgcggtagca

SEQ ID NO: 150 - DNA, 20 bp, synthetic construct
  misc_feature 1..20: note = Synthetic Construct
  source 1..20: mol_type = other DNA
tgcagcatgcctgtgcggta

SEQ ID NO: 151 - DNA, 20 bp, synthetic construct
  misc_feature 1..20: note = Synthetic Construct
  source 1..20: mol_type = other DNA
tcactgcagcatgcctgtgc

SEQ ID NO: 152 - DNA, 20 bp, synthetic construct
  misc_feature 1..20: note = Synthetic Construct
  source 1..20: mol_type = other DNA
ttcactgcagcatgcctgtg

SEQ ID NO: 153 - DNA, 20 bp, synthetic construct
  misc_feature 1..20: note = Synthetic Construct
  source 1..20: mol_type = other DNA
attcactgcagcatgcctgt

SEQ ID NO: 154 - DNA, 20 bp, synthetic construct
  misc_feature 1..20: note = Synthetic Construct
  source 1..20: mol_type = other DNA
aattcactgcagcatgcctg

SEQ ID NO: 155 - DNA, 20 bp, synthetic construct
  misc_feature 1..20: note = Synthetic Construct
  source 1..20: mol_type = other DNA
aaattcactgcagcatgcct

SEQ ID NO: 156 - DNA, 20 bp, synthetic construct
  misc_feature 1..20: note = Synthetic Construct
  source 1..20: mol_type = other DNA
taaattcactgcagcatgcc

SEQ ID NO: 157 - DNA, 20 bp, synthetic construct
  misc_feature 1..20: note = Synthetic Construct
  source 1..20: mol_type = other DNA
ttaaattcactgcagcatgc

SEQ ID NO: 158 - DNA, 20 bp, synthetic construct
  misc_feature 1..20: note = Synthetic Construct
  source 1..20: mol_type = other DNA
gttaaattcactgcagcatg

SEQ ID NO: 159 - DNA, 20 bp, synthetic construct
  misc_feature 1..20: note = Synthetic Construct
  source 1..20: mol_type = other DNA
ggatcagttaaattcactgc

SEQ ID NO: 160 - DNA, 20 bp, synthetic construct
  misc_feature 1..20: note = Synthetic Construct
  source 1..20: mol_type = other DNA
ttaaacaacggttgcaggga

SEQ ID NO: 161 - DNA, 20 bp, synthetic construct
  misc_feature 1..20: note = Synthetic Construct
  source 1..20: mol_type = other DNA
cttaaacaacggttgcaggg

SEQ ID NO: 162 - RNA, 19 nt, synthetic construct
  misc_feature 1..19: note = Synthetic Construct
  source 1..19: mol_type = other RNA
aaaagactgtggaggaaga

SEQ ID NO: 163 - RNA, 20 nt, synthetic construct
  misc_feature 1..20: note = Synthetic Construct
  source 1..20: mol_type = other RNA
aaaagactgtggaggaagaa

SEQ ID NO: 164 - RNA, 19 nt, synthetic construct
  misc_feature 1..19: note = Synthetic Construct
  source 1..19: mol_type = other RNA
aaagactgtggaggaagaa

SEQ ID NO: 165 - RNA, 20 nt, synthetic construct
  misc_feature 1..20: note = Synthetic Construct
  source 1..20: mol_type = other RNA
aaagactgtggaggaagaaa

SEQ ID NO: 166 - RNA, 20 nt, synthetic construct
  misc_feature 1..20: note = Synthetic Construct
  source 1..20: mol_type = other RNA
aagactgtggaggaagaaaa

SEQ ID NO: 167 - RNA, 20 nt, synthetic construct
  misc_feature 1..20: note = Synthetic Construct
  source 1..20: mol_type = other RNA
agactgtggaggaagaaaac

SEQ ID NO: 168 - RNA, 18 nt, synthetic construct
  misc_feature 1..18: note = Synthetic Construct
  source 1..18: mol_type = other RNA
actgtggaggaagaaaac

SEQ ID NO: 169 - RNA, 19 nt, synthetic construct
  misc_feature 1..19: note = Synthetic Construct
  source 1..19: mol_type = other RNA
actgtggaggaagaaaacc

SEQ ID NO: 170 - RNA, 20 nt, synthetic construct
  misc_feature 1..20: note = Synthetic Construct
  source 1..20: mol_type = other RNA
actgtggaggaagaaaaccc

SEQ ID NO: 171 - RNA, 18 nt, synthetic construct
  misc_feature 1..18: note = Synthetic Construct
  source 1..18: mol_type = other RNA
ctgtggaggaagaaaacc

SEQ ID NO: 172 - RNA, 19 nt, synthetic construct
  misc_feature 1..19: note = Synthetic Construct
  source 1..19: mol_type = other RNA
ctgtggaggaagaaaaccc

SEQ ID NO: 173 - RNA, 19 nt, synthetic construct
  misc_feature 1..19: note = Synthetic Construct
  source 1..19: mol_type = other RNA
aaaaccctttaccctgttg

SEQ ID NO: 174 - RNA, 20 nt, synthetic construct
  misc_feature 1..20: note = Synthetic Construct
  source 1..20: mol_type = other RNA
aaaaccctttaccctgttgt

SEQ ID NO: 175 - RNA, 20 nt, synthetic construct
  misc_feature 1..20: note = Synthetic Construct
  source 1..20: mol_type = other RNA
aaaccctttaccctgttgtt

SEQ ID NO: 176 - RNA, 18 nt, synthetic construct
  misc_feature 1..18: note = Synthetic Construct
  source 1..18: mol_type = other RNA
ttgttcagggagaaactg

SEQ ID NO: 177 - RNA, 20 nt, synthetic construct
  misc_feature 1..20: note = Synthetic Construct
  source 1..20: mol_type = other RNA
ttgttcagggagaaactgac

SEQ ID NO: 178 - RNA, 18 nt, synthetic construct
  misc_feature 1..18: note = Synthetic Construct
  source 1..18: mol_type = other RNA
tgttcagggagaaactga

SEQ ID NO: 179 - RNA, 19 nt, synthetic construct
  misc_feature 1..19: note = Synthetic Construct
  source 1..19: mol_type = other RNA
tgttcagggagaaactgac

SEQ ID NO: 180 - RNA, 20 nt, synthetic construct
  misc_feature 1..20: note = Synthetic Construct
  source 1..20: mol_type = other RNA
tgttcagggagaaactgaca

SEQ ID NO: 181 - RNA, 19 nt, synthetic construct
  misc_feature 1..19: note = Synthetic Construct
  source 1..19: mol_type = other RNA
gttcagggagaaactgaca

SEQ ID NO: 182 - RNA, 20 nt, synthetic construct
  misc_feature 1..20: note = Synthetic Construct
  source 1..20: mol_type = other RNA
tcagggagaaactgacacca

SEQ ID NO: 183 - RNA, 19 nt, synthetic construct
  misc_feature 1..19: note = Synthetic Construct
  source 1..19: mol_type = other RNA
cagggagaaactgacacca

SEQ ID NO: 184 - RNA, 18 nt, synthetic construct
  misc_feature 1..18: note = Synthetic Construct
  source 1..18: mol_type = other RNA
agggagaaactgacacca

SEQ ID NO: 185 - RNA, 19 nt, synthetic construct
  misc_feature 1..19: note = Synthetic Construct
  source 1..19: mol_type = other RNA
agggagaaactgacaccac

SEQ ID NO: 186 - RNA, 20 nt, synthetic construct
  misc_feature 1..20: note = Synthetic Construct
  source 1..20: mol_type = other RNA
agggagaaactgacaccact

SEQ ID NO: 187 - RNA, 18 nt, synthetic construct
  misc_feature 1..18: note = Synthetic Construct
  source 1..18: mol_type = other RNA
gggagaaactgacaccac

SEQ ID NO: 188 - RNA, 19 nt, synthetic construct
  misc_feature 1..19: note = Synthetic Construct
  source 1..19: mol_type = other RNA
gggagaaactgacaccact

SEQ ID NO: 189 - RNA, 20 nt, synthetic construct
  misc_feature 1..20: note = Synthetic Construct
  source 1..20: mol_type = other RNA
gggagaaactgacaccactc

SEQ ID NO: 190 - RNA, 18 nt, synthetic construct
  misc_feature 1..18: note = Synthetic Construct
  source 1..18: mol_type = other RNA
ggagaaactgacaccact

SEQ ID NO: 191 - RNA, 19 nt, synthetic construct
  misc_feature 1..19: note = Synthetic Construct
  source 1..19: mol_type = other RNA
ggagaaactgacaccactc

SEQ ID NO: 192 - RNA, 20 nt, synthetic construct
  misc_feature 1..20: note = Synthetic Construct
  source 1..20: mol_type = other RNA
ggagaaactgacaccactca

SEQ ID NO: 193 - RNA, 18 nt, synthetic construct
  misc_feature 1..18: note = Synthetic Construct
  source 1..18: mol_type = other RNA
gagaaactgacaccactc

SEQ ID NO: 194 - RNA, 19 nt, synthetic construct
  misc_feature 1..19: note = Synthetic Construct
  source 1..19: mol_type = other RNA
gagaaactgacaccactca

SEQ ID NO: 195 - RNA, 20 nt, synthetic construct
  misc_feature 1..20: note = Synthetic Construct
  source 1..20: mol_type = other RNA
gagaaactgacaccactcaa

SEQ ID NO: 196 - RNA, 18 nt, synthetic construct
  misc_feature 1..18: note = Synthetic Construct
  source 1..18: mol_type = other RNA
agaaactgacaccactca

SEQ ID NO: 197 - RNA, 19 nt, synthetic construct
  misc_feature 1..19: note = Synthetic Construct
  source 1..19: mol_type = other RNA
agaaactgacaccactcaa

SEQ ID NO: 198 - RNA, 20 nt, synthetic construct
  misc_feature 1..20: note = Synthetic Construct
  source 1..20: mol_type = other RNA
agaaactgacaccactcaac

SEQ ID NO: 199 - RNA, 18 nt, synthetic construct
  misc_feature 1..18: note = Synthetic Construct
  source 1..18: mol_type = other RNA
gaaactgacaccactcaa

SEQ ID NO: 200 - RNA, 19 nt, synthetic construct
  misc_feature 1..19: note = Synthetic Construct
  source 1..19: mol_type = other RNA
gaaactgacaccactcaac

SEQ ID NO: 201 - RNA, 20 nt, synthetic construct
  misc_feature 1..20: note = Synthetic Construct
  source 1..20: mol_type = other RNA
gaaactgacaccactcaact

SEQ ID NO: 202 - RNA, 18 nt, synthetic construct
  misc_feature 1..18: note = Synthetic Construct
  source 1..18: mol_type = other RNA
aaactgacaccactcaac

SEQ ID NO: 203 - RNA, 19 nt, synthetic construct
  misc_feature 1..19: note = Synthetic Construct
  source 1..19: mol_type = other RNA
aaactgacaccactcaact

SEQ ID NO: 204 - RNA, 20 nt, synthetic construct
  misc_feature 1..20: note = Synthetic Construct
  source 1..20: mol_type = other RNA
aaactgacaccactcaactg

SEQ ID NO: 205 - RNA, 18 nt, synthetic construct
  misc_feature 1..18: note = Synthetic Construct
  source 1..18: mol_type = other RNA
aactgacaccactcaact

SEQ ID NO: 206 - RNA, 19 nt, synthetic construct
  misc_feature 1..19: note = Synthetic Construct
  source 1..19: mol_type = other RNA
aactgacaccactcaactg

SEQ ID NO: 207 - RNA, 20 nt, synthetic construct
  misc_feature 1..20: note = Synthetic Construct
  source 1..20: mol_type = other RNA
aactgacaccactcaactgc

SEQ ID NO: 208 - RNA, 18 nt, synthetic construct
  misc_feature 1..18: note = Synthetic Construct
  source 1..18: mol_type = other RNA
actgacaccactcaactg

SEQ ID NO: 209 - RNA, 19 nt, synthetic construct
  misc_feature 1..19: note = Synthetic Construct
  source 1..19: mol_type = other RNA
actgacaccactcaactgc

SEQ ID NO: 210 - RNA, 20 nt, synthetic construct
  misc_feature 1..20: note = Synthetic Construct
  source 1..20: mol_type = other RNA
actgacaccactcaactgcc

SEQ ID NO: 211 - RNA, 18 nt, synthetic construct
  misc_feature 1..18: note = Synthetic Construct
  source 1..18: mol_type = other RNA
ctgacaccactcaactgc

SEQ ID NO: 212 - RNA, 19 nt, synthetic construct
  misc_feature 1..19: note = Synthetic Construct
  source 1..19: mol_type = other RNA
ctgacaccactcaactgcc

SEQ ID NO: 213 - RNA, 20 nt, synthetic construct
  misc_feature 1..20: note = Synthetic Construct
  source 1..20: mol_type = other RNA
ctgacaccactcaactgcct

SEQ ID NO: 214 - RNA, 18 nt, synthetic construct
  misc_feature 1..18: note = Synthetic Construct
  source 1..18: mol_type = other RNA
tgacaccactcaactgcc

SEQ ID NO: 215 - RNA, 19 nt, synthetic construct
  misc_feature 1..19: note = Synthetic Construct
  source 1..19: mol_type = other RNA
tgacaccactcaactgcct

SEQ ID NO: 216 - RNA, 20 nt, synthetic construct
  misc_feature 1..20: note = Synthetic Construct
  source 1..20: mol_type = other RNA
tgacaccactcaactgcctg

SEQ ID NO: 217 - RNA, 18 nt, synthetic construct
  misc_feature 1..18: note = Synthetic Construct
  source 1..18: mol_type = other RNA
gacaccactcaactgcct

SEQ ID NO: 218 - RNA, 19 nt, synthetic construct
  misc_feature 1..19: note = Synthetic Construct
  source 1..19: mol_type = other RNA
gacaccactcaactgcctg

SEQ ID NO: 219 - RNA, 20 nt, synthetic construct
  misc_feature 1..20: note = Synthetic Construct
  source 1..20: mol_type = other RNA
gacaccactcaactgcctgg

SEQ ID NO: 220 - RNA, 18 nt, synthetic construct
  misc_feature 1..18: note = Synthetic Construct
  source 1..18: mol_type = other RNA
acaccactcaactgcctg

SEQ ID NO: 221 - RNA, 19 nt, synthetic construct
  misc_feature 1..19: note = Synthetic Construct
  source 1..19: mol_type = other RNA
acaccactcaactgcctgg

SEQ ID NO: 222 - RNA, 20 nt, synthetic construct
  misc_feature 1..20: note = Synthetic Construct
  source 1..20: mol_type = other RNA
acaccactcaactgcctggc

SEQ ID NO: 223 - RNA, 20 nt, synthetic construct
  misc_feature 1..20: note = Synthetic Construct
  source 1..20: mol_type = other RNA
caccactcaactgcctggca

SEQ ID NO: 224 - RNA, 18 nt, synthetic construct
  misc_feature 1..18: note = Synthetic Construct
  source 1..18: mol_type = other RNA
gaaaatgtggcatccagt

SEQ ID NO: 225 - RNA, 18 nt, synthetic construct
  misc_feature 1..18: note = Synthetic Construct
  source 1..18: mol_type = other RNA
aaaatgtggcatccagtc

SEQ ID NO: 226 - RNA, 20 nt, synthetic construct
  misc_feature 1..20: note = Synthetic Construct
  source 1..20: mol_type = other RNA
gcatccagtccactttacca

SEQ ID NO: 227 - RNA, 18 nt, synthetic construct
  misc_feature 1..18: note = Synthetic Construct
  source 1..18: mol_type = other RNA
catccagtccactttacc

SEQ ID NO: 228 - RNA, 19 nt, synthetic construct
  misc_feature 1..19: note = Synthetic Construct
  source 1..19: mol_type = other RNA
catccagtccactttacca

SEQ ID NO: 229 - RNA, 20 nt, synthetic construct
  misc_feature 1..20: note = Synthetic Construct
  source 1..20: mol_type = other RNA
catccagtccactttaccat

SEQ ID NO: 230 - RNA, 18 nt, synthetic construct
  misc_feature 1..18: note = Synthetic Construct
  source 1..18: mol_type = other RNA
atccagtccactttacca

SEQ ID NO: 231 - RNA, 19 nt, synthetic construct
  misc_feature 1..19: note = Synthetic Construct
  source 1..19: mol_type = other RNA
atccagtccactttaccat

SEQ ID NO: 232 - RNA, 20 nt, synthetic construct
  misc_feature 1..20: note = Synthetic Construct
  source 1..20: mol_type = other RNA
atccagtccactttaccatc

SEQ ID NO: 233 - RNA, 20 nt, synthetic construct
  misc_feature 1..20: note = Synthetic Construct
  source 1..20: mol_type = other RNA
gtttaaggaaaccatctctg

SEQ ID NO: 234 - RNA, 20 nt, synthetic construct
  misc_feature 1..20: note = Synthetic Construct
  source 1..20: mol_type = other RNA
tttaaggaaaccatctctgg

SEQ ID NO: 235 - RNA, 19 nt, synthetic construct
  misc_feature 1..19: note = Synthetic Construct
  source 1..19: mol_type = other RNA
ttaaggaaaccatctctgg

SEQ ID NO: 236 - RNA, 18 nt, synthetic construct
  misc_feature 1..18: note = Synthetic Construct
  source 1..18: mol_type = other RNA
taaggaaaccatctctgg

SEQ ID NO: 237 - DNA, 19 bp, synthetic construct
  misc_feature 1..19: note = Synthetic Construct
  source 1..19: mol_type = other DNA
tcttcctccacagtctttt

SEQ ID NO: 238 - DNA, 20 bp, synthetic construct
  misc_feature 1..20: note = Synthetic Construct
  source 1..20: mol_type = other DNA
ttcttcctccacagtctttt

SEQ ID NO: 239 - DNA, 19 bp, synthetic construct
  misc_feature 1..19: note = Synthetic Construct
  source 1..19: mol_type = other DNA
ttcttcctccacagtcttt

SEQ ID NO: 240 - DNA, 20 bp, synthetic construct
  misc_feature 1..20: note = Synthetic Construct
  source 1..20: mol_type = other DNA
tttcttcctccacagtcttt

SEQ ID NO: 241 - DNA, 20 bp, synthetic construct
  misc_feature 1..20: note = Synthetic Construct
  source 1..20: mol_type = other DNA
ttttcttcctccacagtctt

SEQ ID NO: 242 - DNA, 20 bp, synthetic construct
  misc_feature 1..20: note = Synthetic Construct
  source 1..20: mol_type = other DNA
gttttcttcctccacagtct

SEQ ID NO: 243 - DNA, 18 bp, synthetic construct
  misc_feature 1..18: note = Synthetic Construct
  source 1..18: mol_type = other DNA
gttttcttcctccacagt

SEQ ID NO: 244 - DNA, 19 bp, synthetic construct
  misc_feature 1..19: note = Synthetic Construct
  source 1..19: mol_type = other DNA
ggttttcttcctccacagt

SEQ ID NO: 245 - DNA, 20 bp, synthetic construct
  misc_feature 1..20: note = Synthetic Construct
  source 1..20: mol_type = other DNA
gggttttcttcctccacagt

SEQ ID NO: 246 - DNA, 18 bp, synthetic construct
  misc_feature 1..18: note = Synthetic Construct
  source 1..18: mol_type = other DNA
ggttttcttcctccacag

SEQ ID NO: 247 - DNA, 19 bp, synthetic construct
  misc_feature 1..19: note = Synthetic Construct
  source 1..19: mol_type = other DNA
gggttttcttcctccacag

SEQ ID NO: 248 - DNA, 19 bp, synthetic construct
  misc_feature 1..19: note = Synthetic Construct
  source 1..19: mol_type = other DNA
caacagggtaaagggtttt

SEQ ID NO: 249 - DNA, 20 bp, synthetic construct
  misc_feature 1..20: note = Synthetic Construct
  source 1..20: mol_type = other DNA
acaacagggtaaagggtttt

SEQ ID NO: 250 - DNA, 20 bp, synthetic construct
  misc_feature 1..20: note = Synthetic Construct
  source 1..20: mol_type = other DNA
aacaacagggtaaagggttt

SEQ ID NO: 251 - DNA, 18 bp, synthetic construct
  misc_feature 1..18: note = Synthetic Construct
  source 1..18: mol_type = other DNA
cagtttctccctgaacaa

SEQ ID NO: 252 - DNA, 20 bp, synthetic construct
  misc_feature 1..20: note = Synthetic Construct
  source 1..20: mol_type = other DNA
gtcagtttctccctgaacaa

SEQ ID NO: 253 - DNA, 18 bp, synthetic construct
  misc_feature 1..18: note = Synthetic Construct
  source 1..18: mol_type = other DNA
tcagtttctccctgaaca

SEQ ID NO: 254 - DNA, 19 bp, synthetic construct
  misc_feature 1..19: note = Synthetic Construct
  source 1..19: mol_type = other DNA
gtcagtttctccctgaaca

SEQ ID NO: 255 - DNA, 20 bp, synthetic construct
  misc_feature 1..20: note = Synthetic Construct
  source 1..20: mol_type = other DNA
tgtcagtttctccctgaaca

SEQ ID NO: 256 - DNA, 19 bp, synthetic construct
  misc_feature 1..19: note = Synthetic Construct
  source 1..19: mol_type = other DNA
tgtcagtttctccctgaac

SEQ ID NO: 257 - DNA, 20 bp, synthetic construct
  misc_feature 1..20: note = Synthetic Construct
  source 1..20: mol_type = other DNA
tggtgtcagtttctccctga

SEQ ID NO: 258 - DNA, 19 bp, synthetic construct
  misc_feature 1..19: note = Synthetic Construct
  source 1..19: mol_type = other DNA
tggtgtcagtttctccctg

SEQ ID NO: 259 - DNA, 18 bp, synthetic construct
  misc_feature 1..18: note = Synthetic Construct
  source 1..18: mol_type = other DNA
tggtgtcagtttctccct

SEQ ID NO: 260 - DNA, 19 bp, synthetic construct
  misc_feature 1..19: note = Synthetic Construct
  source 1..19: mol_type = other DNA
gtggtgtcagtttctccct

SEQ ID NO: 261 - DNA, 20 bp, synthetic construct
  misc_feature 1..20: note = Synthetic Construct
  source 1..20: mol_type = other DNA
agtggtgtcagtttctccct

SEQ ID NO: 262 - DNA, 18 bp, synthetic construct
  misc_feature 1..18: note = Synthetic Construct
  source 1..18: mol_type = other DNA
gtggtgtcagtttctccc

SEQ ID NO: 263 - DNA, 19 bp, synthetic construct
  misc_feature 1..19: note = Synthetic Construct
  source 1..19: mol_type = other DNA
agtggtgtcagtttctccc

SEQ ID NO: 264 - DNA, 20 bp, synthetic construct
  misc_feature 1..20: note = Synthetic Construct
  source 1..20: mol_type = other DNA
gagtggtgtcagtttctccc

SEQ ID NO: 265 - DNA, 18 bp, synthetic construct
  misc_feature 1..18: note = Synthetic Construct
  source 1..18: mol_type = other DNA
agtggtgtcagtttctcc

SEQ ID NO: 266 - DNA, 19 bp, synthetic construct
  misc_feature 1..19: note = Synthetic Construct
  source 1..19: mol_type = other DNA
gagtggtgtcagtttctcc

SEQ ID NO: 267 - DNA, 20 bp, synthetic construct
  misc_feature 1..20: note = Synthetic Construct
  source 1..20: mol_type = other DNA
tgagtggtgtcagtttctcc

SEQ ID NO: 268 - DNA, 18 bp, synthetic construct
  misc_feature 1..18: note = Synthetic Construct
  source 1..18: mol_type = other DNA
gagtggtgtcagtttctc

SEQ ID NO: 269 - DNA, 19 bp, synthetic construct
  misc_feature 1..19: note = Synthetic Construct
  source 1..19: mol_type = other DNA
tgagtggtgtcagtttctc

SEQ ID NO: 270 - DNA, 20 bp, synthetic construct
  misc_feature 1..20: note = Synthetic Construct
  source 1..20: mol_type = other DNA
ttgagtggtgtcagtttctc

SEQ ID NO: 271 - DNA, 18 bp, synthetic construct
  misc_feature 1..18: note = Synthetic Construct
  source 1..18: mol_type = other DNA
tgagtggtgtcagtttct

SEQ ID NO: 272 - DNA, 19 bp, synthetic construct
  misc_feature 1..19: note = Synthetic Construct
  source 1..19: mol_type = other DNA
ttgagtggtgtcagtttct

SEQ ID NO: 273 - DNA, 20 bp, synthetic construct
  misc_feature 1..20: note = Synthetic Construct
  source 1..20: mol_type = other DNA
gttgagtggtgtcagtttct

SEQ ID NO: 274 - DNA, 18 bp, synthetic construct
  misc_feature 1..18: note = Synthetic Construct
  source 1..18: mol_type = other DNA
ttgagtggtgtcagtttc

SEQ ID NO: 275 - DNA, 19 bp, synthetic construct
  misc_feature 1..19: note = Synthetic Construct
  source 1..19: mol_type = other DNA
gttgagtggtgtcagtttc

SEQ ID NO: 276 - DNA, 20 bp, synthetic construct
  misc_feature 1..20: note = Synthetic Construct
  source 1..20: mol_type = other DNA
agttgagtggtgtcagtttc

SEQ ID NO: 277 - DNA, 18 bp, synthetic construct
  misc_feature 1..18: note = Synthetic Construct
  source 1..18: mol_type = other DNA
gttgagtggtgtcagttt

SEQ ID NO: 278 - DNA, 19 bp, synthetic construct
  misc_feature 1..19: note = Synthetic Construct
  source 1..19: mol_type = other DNA
agttgagtggtgtcagttt

SEQ ID NO: 279 - DNA, 20 bp, synthetic construct
  misc_feature 1..20: note = Synthetic Construct
  source 1..20: mol_type = other DNA
cagttgagtggtgtcagttt

SEQ ID NO: 280 - DNA, 18 bp, synthetic construct
  misc_feature 1..18: note = Synthetic Construct
  source 1..18: mol_type = other DNA
agttgagtggtgtcagtt

SEQ ID NO: 281 - DNA, 19 bp, synthetic construct
  misc_feature 1..19: note = Synthetic Construct
  source 1..19: mol_type = other DNA
cagttgagtggtgtcagtt

SEQ ID NO: 282 - DNA, 20 bp, synthetic construct
  misc_feature 1..20: note = Synthetic Construct
  source 1..20: mol_type = other DNA
gcagttgagtggtgtcagtt

SEQ ID NO: 283 - DNA, 18 bp, synthetic construct
  misc_feature 1..18: note = Synthetic Construct
  source 1..18: mol_type = other DNA
cagttgagtggtgtcagt

SEQ ID NO: 284 - DNA, 19 bp, synthetic construct
  misc_feature 1..19: note = Synthetic Construct
  source 1..19: mol_type = other DNA
gcagttgagtggtgtcagt

SEQ ID NO: 285 - DNA, 20 bp, synthetic construct
  misc_feature 1..20: note = Synthetic Construct
  source 1..20: mol_type = other DNA
ggcagttgagtggtgtcagt

SEQ ID NO: 286 - DNA, 18 bp, synthetic construct
  misc_feature 1..18: note = Synthetic Construct
  source 1..18: mol_type = other DNA
gcagttgagtggtgtcag

SEQ ID NO: 287 - DNA, 19 bp, synthetic construct
  misc_feature 1..19: note = Synthetic Construct
  source 1..19: mol_type = other DNA
ggcagttgagtggtgtcag

SEQ ID NO: 288 - DNA, 20 bp, synthetic construct
  misc_feature 1..20: note = Synthetic Construct
  source 1..20: mol_type = other DNA
aggcagttgagtggtgtcag

SEQ ID NO: 289 - DNA, 18 bp, synthetic construct
  misc_feature 1..18: note = Synthetic Construct
  source 1..18: mol_type = other DNA
ggcagttgagtggtgtca

SEQ ID NO: 290 - DNA, 19 bp, synthetic construct
  misc_feature 1..19: note = Synthetic Construct
  source 1..19: mol_type = other DNA
aggcagttgagtggtgtca

SEQ ID NO: 291 - DNA, 20 bp, synthetic construct
  misc_feature 1..20: note = Synthetic Construct
  source 1..20: mol_type = other DNA
caggcagttgagtggtgtca

SEQ ID NO: 292 - DNA, 18 bp, synthetic construct
  misc_feature 1..18: note = Synthetic Construct
  source 1..18: mol_type = other DNA
aggcagttgagtggtgtc

SEQ ID NO: 293 - DNA, 19 bp, synthetic construct
  misc_feature 1..19: note = Synthetic Construct
  source 1..19: mol_type = other DNA
caggcagttgagtggtgtc

SEQ ID NO: 294 - DNA, 20 bp, synthetic construct
  misc_feature 1..20: note = Synthetic Construct
  source 1..20: mol_type = other DNA
ccaggcagttgagtggtgtc

SEQ ID NO: 295 - DNA, 18 bp, synthetic construct
  misc_feature 1..18: note = Synthetic Construct
  source 1..18: mol_type = other DNA
caggcagttgagtggtgt

SEQ ID NO: 296 - DNA, 19 bp, synthetic construct
  misc_feature 1..19: note = Synthetic Construct
  source 1..19: mol_type = other DNA
ccaggcagttgagtggtgt

SEQ ID NO: 297 - DNA, 20 bp, synthetic construct
  misc_feature 1..20: note = Synthetic Construct
  source 1..20: mol_type = other DNA
gccaggcagttgagtggtgt

SEQ ID NO: 298 - DNA, 20 bp, synthetic construct
  misc_feature 1..20: note = Synthetic Construct
  source 1..20: mol_type = other DNA
tgccaggcagttgagtggtg

SEQ ID NO: 299 - DNA, 18 bp, synthetic construct
  misc_feature 1..18: note = Synthetic Construct
  source 1..18: mol_type = other DNA
actggatgccacattttc

SEQ ID NO: 300 - DNA, 18 bp, synthetic construct
  misc_feature 1..18: note = Synthetic Construct
  source 1..18: mol_type = other DNA
gactggatgccacatttt

SEQ ID NO: 301 - DNA, 20 bp, synthetic construct
  misc_feature 1..20: note = Synthetic Construct
  source 1..20: mol_type = other DNA
tggtaaagtggactggatgc

SEQ ID NO: 302 - DNA, 18 bp, synthetic construct
  misc_feature 1..18: note = Synthetic Construct
  source 1..18: mol_type = other DNA
ggtaaagtggactggatg

SEQ ID NO: 303 - DNA, 19 bp, synthetic construct
  misc_feature 1..19: note = Synthetic Construct
  source 1..19: mol_type = other DNA
tggtaaagtggactggatg

SEQ ID NO: 304 - DNA, 20 bp, synthetic construct
  misc_feature 1..20: note = Synthetic Construct
  source 1..20: mol_type = other DNA
atggtaaagtggactggatg

SEQ ID NO: 305 - DNA, 18 bp, synthetic construct
  misc_feature 1..18: note = Synthetic Construct
  source 1..18: mol_type = other DNA
tggtaaagtggactggat

SEQ ID NO: 306 - DNA, 19 bp, synthetic construct
  misc_feature 1..19: note = Synthetic Construct
  source 1..19: mol_type = other DNA
atggtaaagtggactggat

SEQ ID NO: 307 - DNA, 20 bp, synthetic construct
  misc_feature 1..20: note = Synthetic Construct
  source 1..20: mol_type = other DNA
gatggtaaagtggactggat

SEQ ID NO: 308 - DNA, 20 bp, synthetic construct
  misc_feature 1..20: note = Synthetic Construct
  source 1..20: mol_type = other DNA
cagagatggtttccttaaac

SEQ ID NO: 309 - DNA, 20 bp, synthetic construct
  misc_feature 1..20: note = Synthetic Construct
  source 1..20: mol_type = other DNA
ccagagatggtttccttaaa

SEQ ID NO: 310 - DNA, 19 bp, synthetic construct
  misc_feature 1..19: note = Synthetic Construct
  source 1..19: mol_type = other DNA
ccagagatggtttccttaa

SEQ ID NO: 311 - DNA, 18 bp, synthetic construct
  misc_feature 1..18: note = Synthetic Construct
  source 1..18: mol_type = other DNA
ccagagatggtttcctta

SEQ ID NO: 312 - RNA, 19 nt, synthetic construct
  misc_feature 1..19: note = Synthetic Construct
  source 1..19: mol_type = other RNA
ataaggatgactgaggaag

SEQ ID NO: 313 - RNA, 20 nt, synthetic construct
  misc_feature 1..20: note = Synthetic Construct
  source 1..20: mol_type = other RNA
ataaggatgactgaggaaga

SEQ ID NO: 314 - RNA, 18 nt, synthetic construct
  misc_feature 1..18: note = Synthetic Construct
  source 1..18: mol_type = other RNA
taaggatgactgaggaag

SEQ ID NO: 315 - RNA, 19 nt, synthetic construct
  misc_feature 1..19: note = Synthetic Construct
  source 1..19: mol_type = other RNA
taaggatgactgaggaaga

SEQ ID NO: 316 - RNA, 20 nt, synthetic construct
  misc_feature 1..20: note = Synthetic Construct
  source 1..20: mol_type = other RNA
taaggatgactgaggaagag

SEQ ID NO: 317 - RNA, 18 nt, synthetic construct
  misc_feature 1..18: note = Synthetic Construct
  source 1..18: mol_type = other RNA
aaggatgactgaggaaga

SEQ ID NO: 318 - RNA, 19 nt, synthetic construct
  misc_feature 1..19: note = Synthetic Construct
  source 1..19: mol_type = other RNA
aaggatgactgaggaagag

SEQ ID NO: 319 - RNA, 20 nt, synthetic construct
  misc_feature 1..20: note = Synthetic Construct
  source 1..20: mol_type = other RNA
aaggatgactgaggaagagt

SEQ ID NO: 320 - RNA, 18 nt, synthetic construct
  misc_feature 1..18: note = Synthetic Construct
  source 1..18: mol_type = other RNA
aggatgactgaggaagag

SEQ ID NO: 321 - RNA, 19 nt, synthetic construct
  misc_feature 1..19: note = Synthetic Construct
  source 1..19: mol_type = other RNA
aggatgactgaggaagagt

SEQ ID NO: 322 - RNA, 20 nt, synthetic construct
  misc_feature 1..20: note = Synthetic Construct
  source 1..20: mol_type = other RNA
aggatgactgaggaagagta

SEQ ID NO: 323 - RNA, 18 nt, synthetic construct
  misc_feature 1..18: note = Synthetic Construct
  source 1..18: mol_type = other RNA
ggatgactgaggaagagt

SEQ ID NO: 324 - RNA, 19 nt, synthetic construct
  misc_feature 1..19: note = Synthetic Construct
  source 1..19: mol_type = other RNA
ggatgactgaggaagagta

SEQ ID NO: 325 - RNA, 20 nt, synthetic construct
  misc_feature 1..20: note = Synthetic Construct
  source 1..20: mol_type = other RNA
ggatgactgaggaagagtac

SEQ ID NO: 326 - RNA, 18 nt, synthetic construct
  misc_feature 1..18: note = Synthetic Construct
  source 1..18: mol_type = other RNA
gatgactgaggaagagta

SEQ ID NO: 327 - RNA, 19 nt, synthetic construct
  misc_feature 1..19: note = Synthetic Construct
  source 1..19: mol_type = other RNA
gatgactgaggaagagtac

SEQ ID NO: 328 - RNA, 20 nt, synthetic construct
  misc_feature 1..20: note = Synthetic Construct
  source 1..20: mol_type = other RNA
gatgactgaggaagagtact

SEQ ID NO: 329 - RNA, 18 nt, synthetic construct
  misc_feature 1..18: note = Synthetic Construct
  source 1..18: mol_type = other RNA
atgactgaggaagagtac

SEQ ID NO: 330 - RNA, 19 nt, synthetic construct
  misc_feature 1..19: note = Synthetic Construct
  source 1..19: mol_type = other RNA
atgactgaggaagagtact

SEQ ID NO: 331 - RNA, 20 nt, synthetic construct
  misc_feature 1..20: note = Synthetic Construct
  source 1..20: mol_type = other RNA
atgactgaggaagagtactc

SEQ ID NO: 332 - RNA, 18 nt, synthetic construct
  misc_feature 1..18: note = Synthetic Construct
  source 1..18: mol_type = other RNA
tgactgaggaagagtact

SEQ ID NO: 333 - RNA, 19 nt, synthetic construct
  misc_feature 1..19: note = Synthetic Construct
  source 1..19: mol_type = other RNA
tgactgaggaagagtactc

SEQ ID NO: 334 - RNA, 20 nt, synthetic construct
  misc_feature 1..20: note = Synthetic Construct
  source 1..20: mol_type = other RNA
tgactgaggaagagtactct

SEQ ID NO: 335 - DNA, 19 bp, synthetic construct
  misc_feature 1..19: note = Synthetic Construct
  source 1..19: mol_type = other DNA
cttcctcagtcatccttat

SEQ ID NO: 336 - DNA, 20 bp, synthetic construct
  misc_feature 1..20: note = Synthetic Construct
  source 1..20: mol_type = other DNA
tcttcctcagtcatccttat

SEQ ID NO: 337 - DNA, 18 bp, synthetic construct
  misc_feature 1..18: note = Synthetic Construct
  source 1..18: mol_type = other DNA
cttcctcagtcatcctta

SEQ ID NO: 338 - DNA, 19 bp, synthetic construct
  misc_feature 1..19: note = Synthetic Construct
  source 1..19: mol_type = other DNA
tcttcctcagtcatcctta

SEQ ID NO: 339 - DNA, 20 bp, synthetic construct
  misc_feature 1..20: note = Synthetic Construct
  source 1..20: mol_type = other DNA
ctcttcctcagtcatcctta

SEQ ID NO: 340 - DNA, 18 bp, synthetic construct
  misc_feature 1..18: note = Synthetic Construct
  source 1..18: mol_type = other DNA
tcttcctcagtcatcctt

SEQ ID NO: 341 - DNA, 19 bp, synthetic construct
  misc_feature 1..19: note = Synthetic Construct
  source 1..19: mol_type = other DNA
ctcttcctcagtcatcctt

SEQ ID NO: 342 - DNA, 20 bp, synthetic construct
  misc_feature 1..20: note = Synthetic Construct
  source 1..20: mol_type = other DNA
actcttcctcagtcatcctt

SEQ ID NO: 343 - DNA, 18 bp, synthetic construct
  misc_feature 1..18: note = Synthetic Construct
  source 1..18: mol_type = other DNA
ctcttcctcagtcatcct

SEQ ID NO: 344 - DNA, 19 bp, synthetic construct
  misc_feature 1..19: note = Synthetic Construct
  source 1..19: mol_type = other DNA
actcttcctcagtcatcct

SEQ ID NO: 345 - DNA, 20 bp, synthetic construct
  misc_feature 1..20: note = Synthetic Construct
  source 1..20: mol_type = other DNA
tactcttcctcagtcatcct

SEQ ID NO: 346 - DNA, 18 bp, synthetic construct
  misc_feature 1..18: note = Synthetic Construct
  source 1..18: mol_type = other DNA
actcttcctcagtcatcc

SEQ ID NO: 347 - DNA, 19 bp, synthetic construct
  misc_feature 1..19: note = Synthetic Construct
  source 1..19: mol_type = other DNA
tactcttcctcagtcatcc

SEQ ID NO: 348 - DNA, 20 bp, synthetic construct
  misc_feature 1..20: note = Synthetic Construct
  source 1..20: mol_type = other DNA
gtactcttcctcagtcatcc

SEQ ID NO: 349 - DNA, 18 bp, synthetic construct
  misc_feature 1..18: note = Synthetic Construct
  source 1..18: mol_type = other DNA
tactcttcctcagtcatc

SEQ ID NO: 350 - DNA, 19 bp, synthetic construct
  misc_feature 1..19: note = Synthetic Construct
  source 1..19: mol_type = other DNA
gtactcttcctcagtcatc

SEQ ID NO: 351 - DNA, 20 bp, synthetic construct
  misc_feature 1..20: note = Synthetic Construct
  source 1..20: mol_type = other DNA
agtactcttcctcagtcatc

SEQ ID NO: 352 - DNA, 18 bp, synthetic construct
  misc_feature 1..18: note = Synthetic Construct
  source 1..18: mol_type = other DNA
gtactcttcctcagtcat

SEQ ID NO: 353 - DNA, 19 bp, synthetic construct
  misc_feature 1..19: note = Synthetic Construct
  source 1..19: mol_type = other DNA
agtactcttcctcagtcat

SEQ ID NO: 354 - DNA, 20 bp, synthetic construct
  misc_feature 1..20: note = Synthetic Construct
  source 1..20: mol_type = other DNA
gagtactcttcctcagtcat

SEQ ID NO: 355 - DNA, 18 bp, synthetic construct
  misc_feature 1..18: note = Synthetic Construct
  source 1..18: mol_type = other DNA
agtactcttcctcagtca

SEQ ID NO: 356 - DNA, 19 bp, synthetic construct
  misc_feature 1..19: note = Synthetic Construct
  source 1..19: mol_type = other DNA
gagtactcttcctcagtca

SEQ ID NO: 357 - DNA, 20 bp, synthetic construct
  misc_feature 1..20: note = Synthetic Construct
  source 1..20: mol_type = other DNA
agagtactcttcctcagtca

SEQ ID NO: 358 - DNA, 20 bp, synthetic construct
  misc_feature 1..20: note = Synthetic Construct
  modified_base 1..2: mod_base = cm; note = 2'-O-methylcytidine
  misc_feature 1..20: note = phosphorothioate backbone
  modified_base 3: mod_base = OTHER; note = 2'-O-methyladenosine
  modified_base 4: mod_base = gm; note = 2'-O-methylguanosine
  modified_base 5: mod_base = cm; note = 2'-O-methylcytidine
  modified_base 16: mod_base = OTHER; note = 2'-O-methyladenosine
  modified_base 17: mod_base = um; note = 2'-O-methyluridine
  modified_base 18: mod_base = cm; note = 2'-O-methylcytidine
  modified_base 19: mod_base = OTHER; note = 2'-O-methyladenosine
  modified_base 20: mod_base = um; note = 2'-O-methyluridine
  source 1..20: mol_type = other DNA
nnnnncttgttggatnnnnn

SEQ ID NO: 359 - DNA, 20 bp, synthetic construct
  misc_feature 1..20: note = Synthetic Construct
  modified_base 1..2: mod_base = cm; note = 2'-O-methylcytidine
  misc_feature 1..20: note = phosphorothioate backbone
  modified_base 3: mod_base = OTHER; note = 2'-O-methyladenosine
  modified_base 4: mod_base = cm; note = 2'-O-methylcytidine
  modified_base 5: mod_base = OTHER; note = 2'-O-methyladenosine
  modified_base 16: mod_base = um; note = 2'-O-methyluridine
  modified_base 17..18: mod_base = gm; note = 2'-O-methylguanosine
  modified_base 19..20: mod_base = OTHER; note = 2'-O-methyladenosine
  source 1..20: mol_type = other DNA
nnnnntttcctctcannnnn

SEQ ID NO: 360 - DNA, 20 bp, synthetic construct
  misc_feature 1..20: note = Synthetic Construct
  misc_feature 1..20: note = phosphorothioate backbone
  modified_base 1: mod_base = gm; note = 2'-O-methylguanosine
  modified_base 2: mod_base = OTHER; note = 2'-O-methyladenosine
  modified_base 3: mod_base = gm; note = 2'-O-methylguanosine
  modified_base 4: mod_base = um; note = 2'-O-methyluridine
  modified_base 5: mod_base = gm; note = 2'-O-methylguanosine
  modified_base 16..17: mod_base = OTHER; note = 2'-O-methyladenosine
  modified_base 18: mod_base = um; note = 2'-O-methyluridine
  modified_base 19: mod_base = gm; note = 2'-O-methylguanosine
  modified_base 20: mod_base = um; note = 2'-O-methyluridine
  source 1..20: mol_type = other DNA
nnnnntttgcaaaccnnnnn

SEQ ID NO: 361 - DNA, 20 bp, synthetic construct
  misc_feature 1..20: note = Synthetic Construct
  modified_base 1: mod_base = OTHER; note = 2'-O-methyluridine
  misc_feature 1..20: note = phosphorothioate backbone
  modified_base 2: mod_base = OTHER; note = 2'-O-methylguanosine
  modified_base 3..5: mod_base = OTHER; note = 2'-O-methyluridine
  modified_base 16: mod_base = um; note = 2'-O-methyluridine
  modified_base 17: mod_base = OTHER; note = 2'-O-methylcytidine
  modified_base 18: mod_base = OTHER; note = 2'-O-methyluridine
  modified_base 19: mod_base = OTHER; note = 2'-O-methylguanosine
  modified_base 20: mod_base = OTHER; note = 2'-O-methylcytidine
  source 1..20: mol_type = other DNA
nnnnnctttggtgatnnnnn

SEQ ID NO: 362 - DNA, 20 bp, synthetic construct
  misc_feature 1..20: note = Synthetic Construct
  modified_base 1: mod_base = OTHER; note = 2'-O-methyluridine
  misc_feature 1..20: note = phosphorothioate backbone
  modified_base 2: mod_base = OTHER; note = 2'-O-methyladenosine
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methylguanosine
  modified_base 16..17: mod_base = OTHER; note = 2'-O-methylguanosine
  modified_base 18: mod_base = OTHER; note = 2'-O-methylcytidine
  modified_base 19: mod_base = OTHER; note = 2'-O-methyladenosine
  modified_base 20: mod_base = OTHER; note = 2'-O-methylcytidine
  source 1..20: mol_type = other DNA
nnnnngtgaaggccannnnn

SEQ ID NO: 363 - DNA, 20 bp, synthetic construct
  misc_feature 1..20: note = Synthetic Construct
  modified_base 1: mod_base = OTHER; note = 2'-O-methylguanosine
  misc_feature 1..20: note = phosphorothioate backbone
  modified_base 2: mod_base = OTHER; note = 2'-O-methyluridine
  modified_base 3: mod_base = OTHER; note = 2'-O-methyladenosine
  modified_base 4: mod_base = OTHER; note = 2'-O-methylcytidine
  modified_base 5: mod_base = OTHER; note = 2'-O-methyluridine
  modified_base 16: mod_base = OTHER; note = 2'-O-methylcytidine
  modified_base 17: mod_base = OTHER; note = 2'-O-methyladenosine
  modified_base 18: mod_base = OTHER; note = 2'-O-methyluridine
  modified_base 19..20: mod_base = OTHER; note = 2'-O-methylcytidine
  source 1..20: mol_type = other DNA
nnnnncttcctcagtnnnnn

SEQ ID NO: 364 - DNA, 20 bp, synthetic construct
  misc_feature 1..20: note = Synthetic Construct
  modified_base 1: mod_base = OTHER; note = 2'-O-methyluridine
  misc_feature 1..20: note = phosphorothioate backbone
  modified_base 2: mod_base = OTHER; note = 2'-O-methylguanosine
  modified_base 3: mod_base = OTHER; note = 2'-O-methyluridine
  modified_base 4: mod_base = OTHER; note = 2'-O-methylcytidine
  modified_base 5: mod_base = OTHER; note = 2'-O-methyladenosine
  modified_base 16: mod_base = OTHER; note = 2'-O-methylguanosine
  modified_base 17..18: mod_base = OTHER; note = 2'-O-methyladenosine
  modified_base 19: mod_base = OTHER; note = 2'-O-methylcytidine
  modified_base 20: mod_base = OTHER; note = 2'-O-methyladenosine
  source 1..20: mol_type = other DNA
nnnnngtttctccctnnnnn

SEQ ID NO: 365 - DNA, 20 bp, synthetic construct
  misc_feature 1..20: note = Synthetic Construct
  modified_base 1: mod_base = OTHER; note = 2'-O-methyluridine
  misc_feature 1..20: note = phosphorothioate backbone
  modified_base 2: mod_base = OTHER; note = 2'-O-methyladenosine
  modified_base 3: mod_base = OTHER; note = 2'-O-methylguanosine
  modified_base 4..5: mod_base = OTHER; note = 2'-O-methyladenosine
  modified_base 16: mod_base = OTHER; note = 2'-O-methylcytidine
  modified_base 17..18: mod_base = OTHER; note = 2'-O-methyluridine
  modified_base 19..20: mod_base = OTHER; note = 2'-O-methylguanosine
  source 1..20: mol_type = other DNA
nnnnntggcacatctnnnnn

SEQ ID NO: 366 - DNA, 20 bp, synthetic construct
  misc_feature 1..20: note = Synthetic Construct
  modified_base 1: mod_base = OTHER; note = 2'-O-methylguanosine
  misc_feature 1..20: note = phosphorothioate backbone
  modified_base 2..5: mod_base = OTHER; note = 2'-O-methyluridine
  modified_base 16: mod_base = OTHER; note = 2'-O-methyladenosine
  modified_base 17: mod_base = OTHER; note = 2'-O-methylguanosine
  modified_base 18: mod_base = OTHER; note = 2'-O-methyluridine
  modified_base 19: mod_base = OTHER; note = 2'-O-methylcytidine
  modified_base 20: mod_base = OTHER; note = 2'-O-methyluridine
  source 1..20: mol_type = other DNA
nnnnncttcctccacnnnnn

SEQ ID NO: 367 - DNA, 20 bp, synthetic construct
  misc_feature 1..20: note = Synthetic Construct
  modified_base 1: mod_base = OTHER; note = 2'-O-methylcytidine
  misc_feature 1..20: note = phosphorothioate backbone
  modified_base 2: mod_base = OTHER; note = 2'-O-methyluridine
  modified_base 3..4: mod_base = OTHER; note = 2'-O-methylguanosine
  modified_base 5: mod_base = OTHER; note = 2'-O-methyluridine
  modified_base 16: mod_base = OTHER; note = 2'-O-methylcytidine
  modified_base 17..19: mod_base = OTHER; note = 2'-O-methyladenosine
  modified_base 20: mod_base = OTHER; note = 2'-O-methylguanosine
  source 1..20: mol_type = other DNA
nnnnngtcaacaagcnnnnn

SEQ ID NO: 368 - DNA, 19 bp, synthetic construct
  misc_feature 1..19: note = Synthetic Construct
  modified_base 1: mod_base = OTHER; note = 2'-O-methylguanosine
  misc_feature 1..19: note = phosphorothioate backbone
  modified_base 2..3: mod_base = OTHER; note = 2'-O-methyluridine
  modified_base 4: mod_base = OTHER; note = 2'-O-methylguanosine
  modified_base 15: mod_base = OTHER; note = 2'-O-methylguanosine
  modified_base 16..18: mod_base = OTHER; note = 2'-O-methyluridine
  modified_base 19: mod_base = OTHER; note = 2'-O-methylcytidine
  source 1..19: mol_type = other DNA
nnnnagtggtgtcannnnn

SEQ ID NO: 369 - DNA, 18 bp, synthetic construct
  misc_feature 1..18: note = Synthetic Construct
  modified_base 1..2: mod_base = OTHER; note = 2'-O-methyluridine
  misc_feature 1..18: note = phosphorothioate backbone
  modified_base 3: mod_base = OTHER; note = 2'-O-methylguanosine
  modified_base 4: mod_base = OTHER; note = 2'-O-methyladenosine
  modified_base 15..17: mod_base = OTHER; note = 2'-O-methyluridine
  modified_base 18: mod_base = OTHER; note = 2'-O-methylcytidine
  source 1..18: mol_type = other DNA
nnnngtggtgtcagnnnn

SEQ ID NO: 370 - DNA, 18 bp, synthetic construct
  misc_feature 1..18: note = Synthetic Construct
  modified_base 1: mod_base = OTHER; note = 2'-O-methylcytidine
  modified_base 1..18: mod_base = OTHER; note = phosphorothioate backbone
  modified_base 2: mod_base = OTHER; note = 2'-O-methyluridine
  modified_base 3..4: mod_base = OTHER; note = 2'-O-methylguanosine
  modified_base 15..16: mod_base = OTHER; note = 2'-O-methylcytidine
  modified_base 17..18: mod_base = OTHER; note = 2'-O-methyladenosine
  source 1..18: mol_type = other DNA
nnnntgtcaacaagnnnn

SEQ ID NO: 371 - DNA, 20 bp, synthetic construct
  misc_feature 1..20: note = Synthetic Construct
  modified_base 1: mod_base = OTHER; note = 2'-O-methyladenosine
  misc_feature 1..20: note = phosphorothioate backbone
  modified_base 2: mod_base = OTHER; note = 2'-O-methyluridine
  modified_base 3: mod_base = OTHER; note = 2'-O-methyladenosine
  modified_base 4: mod_base = OTHER; note = 2'-O-methylguanosine
  modified_base 5: mod_base = OTHER; note = 2'-O-methyladenosine
  modified_base 16: mod_base = OTHER; note = 2'-O-methyluridine
  modified_base 17: mod_base = OTHER; note = 2'-O-methylcytidine
  modified_base 18..19: mod_base = OTHER; note = 2'-O-methyluridine
  modified_base 20: mod_base = OTHER; note = 2'-O-methylguanosine
  source 1..20: mol_type = other DNA
nnnnnatggcacatcnnnnn

SEQ ID NO: 372 - DNA, 19 bp, synthetic construct
  misc_feature 1..19: note = Synthetic Construct
  modified_base 1: mod_base = OTHER; note = 2'-O-methyladenosine
  misc_feature 1..19: note = phosphorothioate backbone
  modified_base 2: mod_base = OTHER; note = 2'-O-methylguanosine
  modified_base 3..4: mod_base = OTHER; note = 2'-O-methyladenosine
  modified_base 15: mod_base = OTHER; note = 2'-O-methylcytidine
  modified_base 16..17: mod_base = OTHER; note = 2'-O-methyluridine
  modified_base 18..19: mod_base = OTHER; note = 2'-O-methylguanosine
  source 1..19: mol_type = other DNA
nnnntggcacatctnnnnn

SEQ ID NO: 373 - DNA, 19 bp, synthetic construct
  misc_feature 1..19: note = Synthetic Construct
  modified_base 1: mod_base = OTHER; note = 2'-O-methyluridine
  misc_feature 1..19: note = phosphorothioate backbone
  modified_base 2: mod_base = OTHER; note = 2'-O-methyladenosine
  modified_base 3: mod_base = OTHER; note = 2'-O-methylguanosine
  modified_base 4: mod_base = OTHER; note = 2'-O-methyladenosine
  modified_base 15: mod_base = OTHER; note = 2'-O-methyluridine
  modified_base 16: mod_base = OTHER; note = 2'-O-methylcytidine
  modified_base 17..18: mod_base = OTHER; note = 2'-O-methyluridine
  modified_base 19: mod_base = OTHER; note = 2'-O-methylguanosine
  source 1..19: mol_type = other DNA
nnnnatggcacatcnnnnn

SEQ ID NO: 374 - DNA, 18 bp, synthetic construct
  misc_feature 1..18: note = Synthetic Construct
  modified_base 1: mod_base = OTHER; note = 2'-O-methyladenosine
  misc_feature 1..18: note = phosphorothioate backbone
  modified_base 2: mod_base = OTHER; note = 2'-O-methylguanosine
  modified_base 3..4: mod_base = OTHER; note = 2'-O-methyladenosine
  modified_base 15: mod_base = OTHER; note = 2'-O-methylcytidine
  modified_base 16..17: mod_base = OTHER; note = 2'-O-methyluridine
  modified_base 18: mod_base = OTHER; note = 2'-O-methylguanosine
  source 1..18: mol_type = other DNA
nnnntggcacatctnnnn

SEQ ID NO: 375 - DNA, 20 bp, synthetic construct
  misc_feature 1..20: note = Synthetic Construct
  modified_base 1: mod_base = OTHER; note = 2'-O-methyluridine
  misc_feature 1..20: note = phosphorothioate backbone
  modified_base 2: mod_base = OTHER; note = 2'-O-methyladenosine
  modified_base 3: mod_base = OTHER; note = 2'-O-methylguanosine
  modified_base 4..5: mod_base = OTHER; note = 2'-O-methyladenosine
  modified_base 16: mod_base = OTHER; note = 2'-O-methylcytidine
  modified_base 17..18: mod_base = OTHER; note = 2'-O-methyluridine
  modified_base 19..20: mod_base = OTHER; note = 2'-O-methylguanosine
  source 1..20: mol_type = other DNA
nnnnntggcacatctnnnnn

SEQ ID NO: 376 - DNA, 20 bp, synthetic construct
  misc_feature 1..20: note = Synthetic Construct
  modified_base 1: mod_base = OTHER; note = 2'-O-methyluridine
  misc_feature 1..5: note = phosphorothioate backbone
  modified_base 2: mod_base = OTHER; note = 2'-O-methyladenosine
  modified_base 3: mod_base = OTHER; note = 2'-O-methylguanosine
  modified_base 4..5: mod_base = OTHER; note = 2'-O-methyladenosine
  misc_feature 5..6: note = phosphodiester backbone
  misc_feature 6..15: note = phosphorothioate backbone
  misc_feature 15..16: note = phosphodiester backbone
  misc_feature 16..20: note = phosphorothioate backbone
  modified_base 16: mod_base = OTHER; note = 2'-O-methylcytidine
  modified_base 17..18: mod_base = OTHER; note = 2'-O-methyluridine
  modified_base 19..20: mod_base = OTHER; note = 2'-O-methylguanosine
  source 1..20: mol_type = other DNA
nnnnntggcacatctnnnnn

SEQ ID NO: 377 - DNA, 20 bp, synthetic construct
  misc_feature 1..20: note = Synthetic Construct
  modified_base 1: mod_base = OTHER; note = 2'-O-methyluridine
  misc_feature 1..2: note = phosphorothioate backbone
  misc_feature 2..3: note = phosphodiester backbone
  modified_base 2: mod_base = OTHER; note = 2'-O-methyladenosine
  modified_base 3: mod_base = OTHER; note = 2'-O-methylguanosine
  misc_feature 4..5: note = phosphodiester backbone
  modified_base 4..5: mod_base = OTHER; note = 2'-O-methyladenosine
  misc_feature 6..7: note = phosphodiester backbone
  misc_feature 8..9: note = phosphodiester backbone
  misc_feature 10..11: note = phosphodiester backbone
  misc_feature 12..13: note = phosphodiester backbone
  misc_feature 14..15: note = phosphodiester backbone
  misc_feature 16..17: note = phosphodiester backbone
  modified_base 16: mod_base = OTHER; note = 2'-O-methylcytidine
  modified_base 17..18: mod_base = OTHER; note = 2'-O-methyluridine
  misc_feature 18..19: note = phosphodiester backbone
  modified_base 19..20: mod_base = OTHER; note = 2'-O-methylguanosine
  source 1..20: mol_type = other DNA
  misc_feature 3..4: note = phosphorothioate backbone
  misc_feature 5..6: note = phosphorothioate backbone
  misc_feature 7..8: note = phosphorothioate backbone
  misc_feature 9..10: note = phosphorothioate backbone
  misc_feature 11..12: note = phosphorothioate backbone
  misc_feature 13..14: note = phosphorothioate backbone
  misc_feature 15..16: note = phosphorothioate backbone
  misc_feature 17..18: note = phosphorothioate backbone
  misc_feature 19..20: note = phosphorothioate backbone
nnnnntggcacatctnnnnn

SEQ ID NO: 378 - DNA, 20 bp, synthetic construct
  misc_feature 1..20: note = Synthetic Construct
  modified_base 1: mod_base = OTHER; note = 2'-O-(2-methoxyethyl)-modified thymidine
  misc_feature 1..20: note = phosphorothioate backbone
  modified_base 2: mod_base = OTHER; note = 2'-O-(2-methoxyethyl)-modified adenosine
  modified_base 3: mod_base = OTHER; note = 2'-O-(2-methoxyethyl)-modified guanosine
  modified_base 4..5: mod_base = OTHER; note = 2'-O-(2-methoxyethyl)-modified adenosine
  modified_base 16: mod_base = OTHER; note = 2'-O-(2-methoxyethyl)-modified 5-methylcytidine
  modified_base 17..18: mod_base = OTHER; note = 2'-O-(2-methoxyethyl)-modified thymidine
  modified_base 19..20: mod_base = OTHER; note = 2'-O-(2-methoxyethyl)-modified guanosine
  source 1..20: mol_type = other DNA
nnnnntggcacatctnnnnn

SEQ ID NO: 379 - DNA, 20 bp, synthetic construct
  misc_feature 1..20: note = Synthetic Construct
  misc_feature 1..5: note = phosphorothioate backbone
  modified_base 1: mod_base = OTHER; note = 2'-O-(2-methoxyethyl)-modified thymidine
  modified_base 2: mod_base = OTHER; note = 2'-O-(2-methoxyethyl)-modified adenosine
  modified_base 3: mod_base = OTHER; note = 2'-O-(2-methoxyethyl)-modified guanosine
  modified_base 4..5: mod_base = OTHER; note = 2'-O-(2-methoxyethyl)-modified adenosine
  misc_feature 5..6: note = phosphodiester backbone
  misc_feature 6..15: note = phosphorothioate backbone
  misc_feature 15..16: note = phosphodiester backbone
  modified_base 16: mod_base = OTHER; note = 2'-O-(2-methoxyethyl)-modified 5-methylcytidine
  misc_feature 16..20: note = phosphorothioate backbone
  modified_base 17..18: mod_base = OTHER; note = 2'-O-(2-methoxyethyl)-modified thymidine
  modified_base 19..20: mod_base = OTHER; note = 2'-O-(2-methoxyethyl)-modified guanosine
  source 1..20: mol_type = other DNA
nnnnntggcacatctnnnnn

SEQ ID NO: 380 - DNA, 20 bp, synthetic construct
  misc_feature 1..20: note = Synthetic Construct
  modified_base 1: mod_base = OTHER; note = 2'-O-(2-methoxyethyl)-modified thymidine
  misc_feature 1..2: note = phosphorothioate backbone
  misc_feature 2..3: note = phosphodiester backbone
  modified_base 2: mod_base = OTHER; note = 2'-O-(2-methoxyethyl)-modified adenosine
  misc_feature 3..4: note = phosphorothioate backbone
  modified_base 3: mod_base = OTHER; note = 2'-O-(2-methoxyethyl)-modified guanosine
  misc_feature 4..5: note = phosphodiester backbone
  modified_base 4..5: mod_base = OTHER; note = 2'-O-(2-methoxyethyl)-modified adenosine
  misc_feature 5..6: note = phosphorothioate backbone
  misc_feature 6..7: note = phosphodiester backbone
  misc_feature 7..8: note = phosphorothioate backbone
  misc_feature 8..9: note = phosphodiester backbone
  misc_feature 9..10: note = phosphorothioate backbone
  misc_feature 10..11: note = phosphodiester backbone
  misc_feature 11..12: note = phosphorothioate backbone
  misc_feature 12..13: note = phosphodiester backbone
  misc_feature 13..14: note = phosphorothioate backbone
  misc_feature 14..15: note = phosphodiester backbone
  misc_feature 15..16: note = phosphorothioate backbone
  misc_feature 16..17: note = phosphodiester backbone
  modified_base 16: mod_base = OTHER; note = 2'-O-(2-methoxyethyl)-modified 5-methylcytidine
  misc_feature 17..18: note = phosphorothioate backbone
  modified_base 17..18: mod_base = OTHER; note = 2'-O-(2-methoxyethyl)-modified thymidine
  misc_feature 18..19: note = phosphodiester backbone
  misc_feature 19..20: note = phosphorothioate backbone
  modified_base 19..20: mod_base = OTHER; note = 2'-O-(2-methoxyethyl)-modified guanosine
  source 1..20: mol_type = other DNA
nnnnntggcacatctnnnnn

SEQ ID NO: 381 - DNA, 18 bp, synthetic construct
  misc_feature 1..18: note = Synthetic Construct
  modified_base 1: mod_base = OTHER; note = locked nucleic acid adenosine
  misc_feature 1..18: note = phosphorothioate backbone
  modified_base 2: mod_base = OTHER; note = locked nucleic acid guanosine
  modified_base 3: mod_base = OTHER; note = locked nucleic acid adenosine
  modified_base 15: mod_base = OTHER; note = locked nucleic acid 5-methylcytidine
  modified_base 16..17: mod_base = OTHER; note = locked nucleic acid thymidine
  modified_base 18: mod_base = OTHER; note = locked nucleic acid guanosine
  source 1..18: mol_type = other DNA
nnnatggcacatctnnnn

SEQ ID NO: 382 - DNA, 18 bp, synthetic construct
  misc_feature 1..18: note = Synthetic Construct
  modified_base 1: mod_base = OTHER; note = locked nucleic acid adenosine
  misc_feature 1..3: note = phosphorothioate backbone
  modified_base 2: mod_base = OTHER; note = locked nucleic acid guanosine
  misc_feature 3..4: note = phosphodiester backbone
  modified_base 3: mod_base = OTHER; note = locked nucleic acid adenosine
  misc_feature 4..14: note = phosphorothioate backbone
  misc_feature 14..15: note = phosphodiester backbone
  misc_feature 15..18: note = phosphorothioate backbone
  modified_base 15: mod_base = OTHER; note = locked nucleic acid 5-methylcytidine
  modified_base 16..17: mod_base = OTHER; note = locked nucleic acid thymidine
  modified_base 18: mod_base = OTHER; note = locked nucleic acid guanosine
  source 1..18: mol_type = other DNA
nnnatggcacatctnnnn

SEQ ID NO: 383 - DNA, 18 bp, synthetic construct
  misc_feature 1..18: note = Synthetic Construct
  modified_base 1: mod_base = OTHER; note = locked nucleic acid adenosine
  misc_feature 1..2: note = phosphorothioate backbone
  misc_feature 2..3: note = phosphodiester backbone
  modified_base 2: mod_base = OTHER; note = locked nucleic acid guanosine
  misc_feature 3..4: note = phosphorothioate backbone
  modified_base 3: mod_base = OTHER; note = locked nucleic acid adenosine
  misc_feature 4..5: note = phosphodiester backbone
  misc_feature 5..6: note = phosphorothioate backbone
  misc_feature 6..7: note = phosphodiester backbone
  misc_feature 7..8: note = phosphorothioate backbone
  misc_feature 8..9: note = phosphodiester backbone
  misc_feature 9..10: note = phosphorothioate backbone
  misc_feature 10..11: note = phosphodiester backbone
  misc_feature 11..12: note = phosphorothioate backbone
  misc_feature 12..13: note = phosphodiester backbone
  misc_feature 13..14: note = phosphorothioate backbone
  misc_feature 14..15: note = phosphodiester backbone
  misc_feature 15..16: note = phosphorothioate backbone
  modified_base 15: mod_base = OTHER; note = locked nucleic acid 5-methylcytidine
  misc_feature 16..17: note = phosphodiester backbone
  modified_base 16..17: mod_base = OTHER; note = locked nucleic acid thymidine
  misc_feature 17..18: note = phosphorothioate backbone
  modified_base 18: mod_base = OTHER; note = locked nucleic acid guanosine
  source 1..18: mol_type = other DNA
nnnatggcacatctnnnn

SEQ ID NO: 384 - DNA, 18 bp, synthetic construct
  misc_feature 1..18: note = Synthetic Construct
  modified_base 1: mod_base = OTHER; note = 2'-O-methylcytidine
  misc_feature 1..18: note = phosphorothioate backbone
  modified_base 2: mod_base = OTHER; note = 2'-O-methyluridine
  modified_base 3..4: mod_base = OTHER; note = 2'-O-methylguanosine
  modified_base 15..16: mod_base = OTHER; note = 2'-O-methylcytidine
  modified_base 17..18: mod_base = OTHER; note = 2'-O-methyladenosine
  source 1..18: mol_type = other DNA
nnnntgtcaacaagnnnn

SEQ ID NO: 385 - DNA, 18 bp, synthetic construct
  misc_feature 1..18: note = Synthetic Construct
  modified_base 1: mod_base = OTHER; note = 2'-O-methylcytidine
  misc_feature 1..4: note = phosphorothioate backbone
  modified_base 2: mod_base = OTHER; note = 2'-O-methyluridine
  modified_base 3..4: mod_base = OTHER; note = 2'-O-methylguanosine
  misc_feature 4..5: note = phosphodiester backbone
  misc_feature 5..14: note = phosphorothioate backbone
  misc_feature 14..15: note = phosphodiester backbone
  misc_feature 15..18: note = phosphorothioate backbone
  modified_base 15..16: mod_base = OTHER; note = 2'-O-methylcytidine
  modified_base 17..18: mod_base = OTHER; note = 2'-O-methyladenosine
  source 1..18: mol_type = other DNA
nnnntgtcaacaagnnnn

SEQ ID NO: 386 - DNA, 18 bp, synthetic construct
  misc_feature 1..18: note = Synthetic Construct
  modified_base 1: mod_base = OTHER; note = 2'-O-methylcytidine
  misc_feature 1..2: note = phosphorothioate backbone
  misc_feature 2..3: note = phosphodiester backbone
  modified_base 2: mod_base = OTHER; note = 2'-O-methyluridine
  misc_feature 3..4: note = phosphorothioate backbone
  modified_base 3..4: mod_base = OTHER; note = 2'-O-methylguanosine
  misc_feature 4..5: note = phosphodiester backbone
  misc_feature 5..6: note = phosphorothioate backbone
  misc_feature 6..7: note = phosphodiester backbone
  misc_feature 7..8: note = phosphorothioate backbone
  misc_feature 8..9: note = phosphodiester backbone
  misc_feature 9..10: note = phosphorothioate backbone
  misc_feature 10..11: note = phosphodiester backbone
  misc_feature 11..12: note = phosphorothioate backbone
  misc_feature 12..13: note = phosphodiester backbone
  misc_feature 13..14: note = phosphorothioate backbone
  misc_feature 14..15: note = phosphodiester backbone
  misc_feature 15..16: note = phosphorothioate backbone
  modified_base 15..16: mod_base = OTHER; note = 2'-O-methylcytidine
  misc_feature 16..17: note = phosphodiester backbone
  misc_feature 17..18: note = phosphorothioate backbone
  modified_base 17..18: mod_base = OTHER; note = 2'-O-methyladenosine
  source 1..18: mol_type = other DNA
nnnntgtcaacaagnnnn

SEQ ID NO: 387 - DNA, 18 bp, synthetic construct
  misc_feature 1..18: note = Synthetic Construct
  modified_base 1: mod_base = OTHER; note = 2'-O-(2-methoxyethyl)-modified 5-methylcytidine
  misc_feature 1..18: note = phosphorothioate backbone
  modified_base 2: mod_base = OTHER; note = 2'-O-(2-methoxyethyl)-modified thymidine
  modified_base 3..4: mod_base = OTHER; note = 2'-O-(2-methoxyethyl)-modified guanosine
  modified_base 15..16: mod_base = OTHER; note = 2'-O-(2-methoxyethyl)-modified 5-methylcytidine
  modified_base 17..18: mod_base = OTHER; note = 2'-O-(2-methoxyethyl)-modified adenosine
  source 1..18: mol_type = other DNA
nnnntgtcaacaagnnnn

SEQ ID NO: 388 - DNA, 18 bp, synthetic construct
  misc_feature 1..18: note = Synthetic Construct
  modified_base 1: mod_base = OTHER; note = 2'-O-(2-methoxyethyl)-modified 5-methylcytidine
  misc_feature 1..4: note = phosphorothioate backbone
  modified_base 2: mod_base = OTHER; note = 2'-O-(2-methoxyethyl)-modified thymidine
  modified_base 3..4: mod_base = OTHER; note = 2'-O-(2-methoxyethyl)-modified guanosine
  misc_feature 4..5: note = phosphodiester backbone
  misc_feature 5..14: note = phosphorothioate backbone
  misc_feature 14..15: note = phosphodiester backbone
  misc_feature 15..18: note = phosphorothioate backbone
  modified_base 15..16: mod_base = OTHER; note = 2'-O-(2-methoxyethyl)-modified 5-methylcytidine
  modified_base 17..18: mod_base = OTHER; note = 2'-O-(2-methoxyethyl)-modified adenosine
  source 1..18: mol_type = other DNA
nnnntgtcaacaagnnnn

SEQ ID NO: 389 - DNA, 18 bp, synthetic construct
  misc_feature 1..18: note = Synthetic Construct
  modified_base 1: mod_base = OTHER; note = 2'-O-(2-methoxyethyl)-modified 5-methylcytidine
  misc_feature 1..2: note = phosphorothioate backbone
  misc_feature 2..3: note = phosphodiester backbone
  modified_base 2: mod_base = OTHER; note = 2'-O-(2-methoxyethyl)-modified thymidine
  misc_feature 3..4: note = phosphorothioate backbone
  modified_base 3..4: mod_base = OTHER; note = 2'-O-(2-methoxyethyl)-modified guanosine
  misc_feature 4..5: note = phosphodiester backbone
  misc_feature 5..6: note = phosphorothioate backbone
  misc_feature 6..7: note = phosphodiester backbone
  misc_feature 7..8: note = phosphorothioate backbone
  misc_feature 8..9: note = phosphodiester backbone
  misc_feature 9..10: note = phosphorothioate backbone
  misc_feature 10..11: note = phosphodiester backbone
  misc_feature 11..12: note = phosphorothioate backbone
  misc_feature 12..13: note = phosphodiester backbone
  misc_feature 13..14: note = phosphorothioate backbone
  misc_feature 14..15: note = phosphodiester backbone
  misc_feature 15..16: note = phosphorothioate backbone
  modified_base 15..16: mod_base = OTHER; note = 2'-O-(2-methoxyethyl)-modified 5-methylcytidine
  misc_feature 16..17: note = phosphodiester backbone
  misc_feature 17..18: note = phosphorothioate backbone
  modified_base 17..18: mod_base = OTHER; note = 2'-O-(2-methoxyethyl)-modified adenosine
  source 1..18: mol_type = other DNA
nnnntgtcaacaagnnnn

SEQ ID NO: 390 - DNA, 17 bp, synthetic construct
  misc_feature 1..17: note = Synthetic Construct
  modified_base 1: mod_base = OTHER; note = locked nucleic acid thymidine
  misc_feature 1..17: note = phosphorothioate backbone
  modified_base 2..3: mod_base = OTHER; note = locked nucleic acid guanosine
  modified_base 14..15: mod_base = OTHER; note = locked nucleic acid 5-methylcytidine
  modified_base 16..17: mod_base = OTHER; note = locked nucleic acid adenosine
  source 1..17: mol_type = other DNA
nnntgtcaacaagnnnn

SEQ ID NO: 391 - DNA, 17 bp, synthetic construct
  misc_feature 1..17: note = Synthetic Construct
  modified_base 1: mod_base = OTHER; note = locked nucleic acid thymidine
  misc_feature 1..3: note = phosphorothioate backbone
  modified_base 2..3: mod_base = OTHER; note = locked nucleic acid guanosine
  misc_feature 3..4: note = phosphodiester backbone
  misc_feature 4..13: note = phosphorothioate backbone
  misc_feature 13..14: note = phosphodiester backbone
  misc_feature 14..17: note = phosphorothioate backbone
  modified_base 14..15: mod_base = OTHER; note = locked nucleic acid 5-methylcytidine
  modified_base 16..17: mod_base = OTHER; note = locked nucleic acid adenosine
  source 1..17: mol_type = other DNA
nnntgtcaacaagnnnn

SEQ ID NO: 392 - DNA, 17 bp, synthetic construct
  misc_feature 1..17: note = Synthetic Construct
  modified_base 1: mod_base = OTHER; note = locked nucleic acid thymidine
  misc_feature 1..2: note = phosphorothioate backbone
  misc_feature 2..3: note = phosphodiester backbone
  modified_base 2..3: mod_base = OTHER; note = locked nucleic acid guanosine
  misc_feature 3..4: note = phosphorothioate backbone
  misc_feature 4..5: note = phosphodiester backbone
  misc_feature 5..6: note = phosphorothioate backbone
  misc_feature 6..7: note = phosphodiester backbone
  misc_feature 7..8: note = phosphorothioate backbone
  misc_feature 8..9: note = phosphodiester backbone
  misc_feature 9..10: note = phosphorothioate backbone
  misc_feature 10..11: note = phosphodiester backbone
  misc_feature 11..12: note = phosphorothioate backbone
  misc_feature 12..13: note = phosphodiester backbone
  misc_feature 13..14: note = phosphorothioate backbone
  misc_feature 14..16: note = phosphodiester backbone
  modified_base 14..15: mod_base = OTHER; note = locked nucleic acid 5-methylcytidine
  misc_feature 16..17: note = phosphorothioate backbone
  modified_base 16..17: mod_base = OTHER; note = locked nucleic acid adenosine
  source 1..17: mol_type = other DNA
nnntgtcaacaagnnnn

SEQ ID NO: 393 - RNA, 20 nt, synthetic construct
  misc_feature 1..20: note = Synthetic Construct
  source 1..20: mol_type = other RNA
gaaaccatctctgggataag

SEQ ID NO: 394 - RNA, 20 nt, synthetic construct
  misc_feature 1..20: note = Synthetic Construct
  source 1..20: mol_type = other RNA
aaaccatctctgggataagg

SEQ ID NO: 395 - RNA, 20 nt, synthetic construct
  misc_feature 1..20: note = Synthetic Construct
  source 1..20: mol_type = other RNA
aaccatctctgggataagga

SEQ ID NO: 396 - RNA, 20 nt, synthetic construct
  misc_feature 1..20: note = Synthetic Construct
  source 1..20: mol_type = other RNA
accatctctgggataaggat

SEQ ID NO: 397 - RNA, 20 nt, synthetic construct
  misc_feature 1..20: note = Synthetic Construct
  source 1..20: mol_type = other RNA
ccatctctgggataaggatg

SEQ ID NO: 398 - RNA, 20 nt, synthetic construct
  misc_feature 1..20: note = Synthetic Construct
  source 1..20: mol_type = other RNA
catctctgggataaggatga

SEQ ID NO: 399 - RNA, 20 nt, synthetic construct
  misc_feature 1..20: note = Synthetic Construct
  source 1..20: mol_type = other RNA
atctctgggataaggatgac

SEQ ID NO: 400 - RNA, 20 nt, synthetic construct
  misc_feature 1..20: note = Synthetic Construct
  source 1..20: mol_type = other RNA
tctctgggataaggatgact

SEQ ID NO: 401 - RNA, 20 nt, synthetic construct
  misc_feature 1..20: note = Synthetic Construct
  source 1..20: mol_type = other RNA
ctctgggataaggatgactg

SEQ ID NO: 402 - RNA, 20 nt, synthetic construct
  misc_feature 1..20: note = Synthetic Construct
  source 1..20: mol_type = other RNA
tctgggataaggatgactga

SEQ ID NO: 403 - RNA, 20 nt, synthetic construct
  misc_feature 1..20: note = Synthetic Construct
  source 1..20: mol_type = other RNA
ctgggataaggatgactgag

SEQ ID NO: 404 - RNA, 20 nt, synthetic construct
  misc_feature 1..20: note = Synthetic Construct
  source 1..20: mol_type = other RNA
tgggataaggatgactgagg

SEQ ID NO: 405 - RNA, 20 nt, synthetic construct
  misc_feature 1..20: note = Synthetic Construct
  source 1..20: mol_type = other RNA
gggataaggatgactgagga

SEQ ID NO: 406 - RNA, 20 nt, synthetic construct
  misc_feature 1..20: note = Synthetic Construct
  source 1..20: mol_type = other RNA
ggataaggatgactgaggaa

SEQ ID NO: 407 - RNA, 20 nt, synthetic construct
  misc_feature 1..20: note = Synthetic Construct
  source 1..20: mol_type = other RNA
gctgaaacaactgaaacttc

SEQ ID NO: 408 - RNA, 20 nt, synthetic construct
  misc_feature 1..20: note = Synthetic Construct
  source 1..20: mol_type = other RNA
gaaacaactgaaacttcagg

SEQ ID NO: 409 - RNA, 20 nt, synthetic construct
  misc_feature 1..20: note = Synthetic Construct
  source 1..20: mol_type = other RNA
aaacaactgaaacttcaggg

SEQ ID NO: 410 - RNA, 20 nt, synthetic construct
  misc_feature 1..20: note = Synthetic Construct
  source 1..20: mol_type = other RNA
aacaactgaaacttcaggga

SEQ ID NO: 411 - RNA, 20 nt, synthetic construct
  misc_feature 1..20: note = Synthetic Construct
  source 1..20: mol_type = other RNA
acaactgaaacttcagggaa

SEQ ID NO: 412 - RNA, 20 nt, synthetic construct
  misc_feature 1..20: note = Synthetic Construct
  source 1..20: mol_type = other RNA
caactgaaacttcagggaaa

SEQ ID NO: 413 - RNA, 20 nt, synthetic construct
  misc_feature 1..20: note = Synthetic Construct
  source 1..20: mol_type = other RNA
actgaaacttcagggaaaag

SEQ ID NO: 414 - RNA, 19 nt, synthetic construct
  misc_feature 1..19: note = Synthetic Construct
  source 1..19: mol_type = other RNA
aaccatctctgggataagg

SEQ ID NO: 415 - RNA, 19 nt, synthetic construct
  misc_feature 1..19: note = Synthetic Construct
  source 1..19: mol_type = other RNA
accatctctgggataagga

SEQ ID NO: 416 - RNA, 19 nt, synthetic construct
  misc_feature 1..19: note = Synthetic Construct
  source 1..19: mol_type = other RNA
ccatctctgggataaggat

SEQ ID NO: 417 - RNA, 19 nt, synthetic construct
  misc_feature 1..19: note = Synthetic Construct
  source 1..19: mol_type = other RNA
catctctgggataaggatg

SEQ ID NO: 418 - RNA, 19 nt, synthetic construct
  misc_feature 1..19: note = Synthetic Construct
  source 1..19: mol_type = other RNA
atctctgggataaggatga

SEQ ID NO: 419 - RNA, 19 nt, synthetic construct
  misc_feature 1..19: note = Synthetic Construct
  source 1..19: mol_type = other RNA
tctctgggataaggatgac

SEQ ID NO: 420 - RNA, 19 nt, synthetic construct
  misc_feature 1..19: note = Synthetic Construct
  source 1..19: mol_type = other RNA
ctctgggataaggatgact

SEQ ID NO: 421 - RNA, 19 nt, synthetic construct
  misc_feature 1..19: note = Synthetic Construct
  source 1..19: mol_type = other RNA
tctgggataaggatgactg

SEQ ID NO: 422 - RNA, 19 nt, synthetic construct
  misc_feature 1..19: note = Synthetic Construct
  source 1..19: mol_type = other RNA
ctgggataaggatgactga

SEQ ID NO: 423 - RNA, 19 nt, synthetic construct
  misc_feature 1..19: note = Synthetic Construct
  source 1..19: mol_type = other RNA
tgggataaggatgactgag

SEQ ID NO: 424 - RNA, 19 nt, synthetic construct
  misc_feature 1..19: note = Synthetic Construct
  source 1..19: mol_type = other RNA
gggataaggatgactgagg

SEQ ID NO: 425 - RNA, 19 nt, synthetic construct
  misc_feature 1..19: note = Synthetic Construct
  source 1..19: mol_type = other RNA
ggataaggatgactgagga

SEQ ID NO: 426 - RNA, 19 nt, synthetic construct
  misc_feature 1..19: note = Synthetic Construct
  source 1..19: mol_type = other RNA
aacaactgaaacttcaggg

SEQ ID NO: 427 - RNA, 19 nt, synthetic construct
  misc_feature 1..19: note = Synthetic Construct
  source 1..19: mol_type = other RNA
acaactgaaacttcaggga

SEQ ID NO: 428 - RNA, 19 nt, synthetic construct
  misc_feature 1..19: note = Synthetic Construct
  source 1..19: mol_type = other RNA
caactgaaacttcagggaa

SEQ ID NO: 429 - RNA, 19 nt, synthetic construct
  misc_feature 1..19: note = Synthetic Construct
  source 1..19: mol_type = other RNA
caactgaaacttcagggaa

SEQ ID NO: 430 - RNA, 18 nt, synthetic construct
  misc_feature 1..18: note = Synthetic Construct
  source 1..18: mol_type = other RNA
ccatctctgggataagga

SEQ ID NO: 431 - RNA, 18 nt, synthetic construct
  misc_feature 1..18: note = Synthetic Construct
  source 1..18: mol_type = other RNA
catctctgggataaggat

SEQ ID NO: 432 - RNA, 18 nt, synthetic construct
  misc_feature 1..18: note = Synthetic Construct
  source 1..18: mol_type = other RNA
atctctgggataaggatg

SEQ ID NO: 433 - RNA, 18 nt, synthetic construct
  misc_feature 1..18: note = Synthetic Construct
  source 1..18: mol_type = other RNA
tctctgggataaggatga

SEQ ID NO: 434 - RNA, 18 nt, synthetic construct
  misc_feature 1..18: note = Synthetic Construct
  source 1..18: mol_type = other RNA
ctctgggataaggatgac

SEQ ID NO: 435 - RNA, 18 nt, synthetic construct
  misc_feature 1..18: note = Synthetic Construct
  source 1..18: mol_type = other RNA
tctgggataaggatgact

SEQ ID NO: 436 - RNA, 18 nt, synthetic construct
  misc_feature 1..18: note = Synthetic Construct
  source 1..18: mol_type = other RNA
ctgggataaggatgactg

SEQ ID NO: 437 - RNA, 18 nt, synthetic construct
  misc_feature 1..18: note = Synthetic Construct
  source 1..18: mol_type = other RNA
tgggataaggatgactga

SEQ ID NO: 438 - RNA, 18 nt, synthetic construct
  misc_feature 1..18: note = Synthetic Construct
  source 1..18: mol_type = other RNA
gggataaggatgactgag

SEQ ID NO: 439 - RNA, 18 nt, synthetic construct
  misc_feature 1..18: note = Synthetic Construct
  source 1..18: mol_type = other RNA
ggataaggatgactgagg

SEQ ID NO: 440 - RNA, 18 nt, synthetic construct
  misc_feature 1..18: note = Synthetic Construct
  source 1..18: mol_type = other RNA
acaactgaaacttcaggg

SEQ ID NO: 441 - DNA, 20 bp, synthetic construct
  misc_feature 1..20: note = Synthetic Construct
  source 1..20: mol_type = other DNA
cttatcccagagatggtttc

SEQ ID NO: 442 - DNA, 20 bp, synthetic construct
  misc_feature 1..20: note = Synthetic Construct
  source 1..20: mol_type = other DNA
ccttatcccagagatggttt

SEQ ID NO: 443 - DNA, 20 bp, synthetic construct
  misc_feature 1..20: note = Synthetic Construct
  source 1..20: mol_type = other DNA
tccttatcccagagatggtt

SEQ ID NO: 444 - DNA, 20 bp, synthetic construct
  misc_feature 1..20: note = Synthetic Construct
  source 1..20: mol_type = other DNA
atccttatcccagagatggt

SEQ ID NO: 445 - DNA, 20 bp, synthetic construct
  misc_feature 1..20: note = Synthetic Construct
  source 1..20: mol_type = other DNA
catccttatcccagagatgg

SEQ ID NO: 446 - DNA, 20 bp, synthetic construct
  misc_feature 1..20: note = Synthetic Construct
  source 1..20: mol_type = other DNA
tcatccttatcccagagatg

SEQ ID NO: 447 - DNA, 20 bp, synthetic construct
  misc_feature 1..20: note = Synthetic Construct
  source 1..20: mol_type = other DNA
gtcatccttatcccagagat

SEQ ID NO: 448 - DNA, 20 bp, synthetic construct
  misc_feature 1..20: note = Synthetic Construct
  source 1..20: mol_type = other DNA
agtcatccttatcccagaga

SEQ ID NO: 449 - DNA, 20 bp, synthetic construct
  misc_feature 1..20: note = Synthetic Construct
  source 1..20: mol_type = other DNA
cagtcatccttatcccagag

SEQ ID NO: 450 - DNA, 20 bp, synthetic construct
  misc_feature 1..20: note = Synthetic Construct
  source 1..20: mol_type = other DNA
tcagtcatccttatcccaga

SEQ ID NO: 451 - DNA, 20 bp, synthetic construct
  misc_feature 1..20: note = Synthetic Construct
  source 1..20: mol_type = other DNA
ctcagtcatccttatcccag

SEQ ID NO: 452 - DNA, 20 bp, synthetic construct
  misc_feature 1..20: note = Synthetic Construct
  source 1..20: mol_type = other DNA
cctcagtcatccttatccca

SEQ ID NO: 453 - DNA, 20 bp, synthetic construct
  misc_feature 1..20: note = Synthetic Construct
  source 1..20: mol_type = other DNA
tcctcagtcatccttatccc

SEQ ID NO: 454 - DNA, 20 bp, synthetic construct
  misc_feature 1..20: note = Synthetic Construct
  source 1..20: mol_type = other DNA
ttcctcagtcatccttatcc

SEQ ID NO: 455 - DNA, 20 bp, synthetic construct
  misc_feature 1..20: note = Synthetic Construct
  source 1..20: mol_type = other DNA
gaagtttcagttgtttcagc

SEQ ID NO: 456 - DNA, 20 bp, synthetic construct
  misc_feature 1..20: note = Synthetic Construct
  source 1..20: mol_type = other DNA
cctgaagtttcagttgtttc

SEQ ID NO: 457 - DNA, 20 bp, synthetic construct
  misc_feature 1..20: note = Synthetic Construct
  source 1..20: mol_type = other DNA
ccctgaagtttcagttgttt

SEQ ID NO: 458 - DNA, 20 bp, synthetic construct
  misc_feature 1..20: note = Synthetic Construct
  source 1..20: mol_type = other DNA
tccctgaagtttcagttgtt

SEQ ID NO: 459 - DNA, 20 bp, synthetic construct
  misc_feature 1..20: note = Synthetic Construct
  source 1..20: mol_type = other DNA
ttccctgaagtttcagttgt

SEQ ID NO: 460 - DNA, 20 bp, synthetic construct
  misc_feature 1..20: note = Synthetic Construct
  source 1..20: mol_type = other DNA
tttccctgaagtttcagttg

SEQ ID NO: 461 - DNA, 20 bp, synthetic construct
  misc_feature 1..20: note = Synthetic Construct
  source 1..20: mol_type = other DNA
cttttccctgaagtttcagt

SEQ ID NO: 462 - DNA, 19 bp, synthetic construct
  misc_feature 1..19: note = Synthetic Construct
  source 1..19: mol_type = other DNA
ccttatcccagagatggtt

SEQ ID NO: 463 - DNA, 19 bp, synthetic construct
  misc_feature 1..19: note = Synthetic Construct
  source 1..19: mol_type = other DNA
tccttatcccagagatggt

SEQ ID NO: 464 - DNA, 19 bp, synthetic construct
  misc_feature 1..19: note = Synthetic Construct
  source 1..19: mol_type = other DNA
atccttatcccagagatgg

SEQ ID NO: 465 - DNA, 19 bp, synthetic construct
  misc_feature 1..19: note = Synthetic Construct
  source 1..19: mol_type = other DNA
catccttatcccagagatg

SEQ ID NO: 466 - DNA, 19 bp, synthetic construct
  misc_feature 1..19: note = Synthetic Construct
  source 1..19: mol_type = other DNA
tcatccttatcccagagat

SEQ ID NO: 467 - DNA, 19 bp, synthetic construct
  misc_feature 1..19: note = Synthetic Construct
  source 1..19: mol_type = other DNA
gtcatccttatcccagaga

SEQ ID NO: 468 - DNA, 19 bp, synthetic construct
  misc_feature 1..19: note = Synthetic Construct
  source 1..19: mol_type = other DNA
agtcatccttatcccagag

SEQ ID NO: 469 - DNA, 19 bp, synthetic construct
  misc_feature 1..19: note = Synthetic Construct
  source 1..19: mol_type = other DNA
cagtcatccttatcccaga

SEQ ID NO: 470 - DNA, 19 bp, synthetic construct
  misc_feature 1..19: note = Synthetic Construct
  source 1..19: mol_type = other DNA
tcagtcatccttatcccag

SEQ ID NO: 471 - DNA, 19 bp, synthetic construct
  misc_feature 1..19: note = Synthetic Construct
  source 1..19: mol_type = other DNA
ctcagtcatccttatccca

SEQ ID NO: 472 - DNA, 19 bp, synthetic construct
  misc_feature 1..19: note = Synthetic Construct
  source 1..19: mol_type = other DNA
cctcagtcatccttatccc

SEQ ID NO: 473 - DNA, 19 bp, synthetic construct
  misc_feature 1..19: note = Synthetic Construct
  source 1..19: mol_type = other DNA
tcctcagtcatccttatcc

SEQ ID NO: 474 - DNA, 19 bp, synthetic construct
  misc_feature 1..19: note = Synthetic Construct
  source 1..19: mol_type = other DNA
ccctgaagtttcagttgtt

SEQ ID NO: 475 - DNA, 19 bp, synthetic construct
  misc_feature 1..19: note = Synthetic Construct
  source 1..19: mol_type = other DNA
tccctgaagtttcagttgt

SEQ ID NO: 476 - DNA, 19 bp, synthetic construct
  misc_feature 1..19: note = Synthetic Construct
  source 1..19: mol_type = other DNA
ttccctgaagtttcagttg

SEQ ID NO: 477 - DNA, 19 bp, synthetic construct
  misc_feature 1..19: note = Synthetic Construct
  source 1..19: mol_type = other DNA
ttccctgaagtttcagttg

SEQ ID NO: 478 - DNA, 18 bp, synthetic construct
  misc_feature 1..18: note = Synthetic Construct
  source 1..18: mol_type = other DNA
tccttatcccagagatgg

SEQ ID NO: 479 - DNA, 18 bp, synthetic construct
  misc_feature 1..18: note = Synthetic Construct
  source 1..18: mol_type = other DNA
atccttatcccagagatg

SEQ ID NO: 480 - DNA, 18 bp, synthetic construct
  misc_feature 1..18: note = Synthetic Construct
  source 1..18: mol_type = other DNA
catccttatcccagagat

SEQ ID NO: 481 - DNA, 18 bp, synthetic construct
  misc_feature 1..18: note = Synthetic Construct
  source 1..18: mol_type = other DNA
tcatccttatcccagaga

SEQ ID NO: 482 - DNA, 18 bp, synthetic construct
  misc_feature 1..18: note = Synthetic Construct
  source 1..18: mol_type = other DNA
gtcatccttatcccagag

SEQ ID NO: 483 - DNA, 18 bp, synthetic construct
  misc_feature 1..18: note = Synthetic Construct
  source 1..18: mol_type = other DNA
agtcatccttatcccaga

SEQ ID NO: 484 - DNA, 18 bp, synthetic construct
  misc_feature 1..18: note = Synthetic Construct
  source 1..18: mol_type = other DNA
cagtcatccttatcccag

SEQ ID NO: 485 - DNA, 18 bp, synthetic construct
  misc_feature 1..18: note = Synthetic Construct
  source 1..18: mol_type = other DNA
tcagtcatccttatccca

SEQ ID NO: 486 - DNA, 18 bp, synthetic construct
  misc_feature 1..18: note = Synthetic Construct
  source 1..18: mol_type = other DNA
ctcagtcatccttatccc

SEQ ID NO: 487 - DNA, 18 bp, synthetic construct
  misc_feature 1..18: note = Synthetic Construct
  source 1..18: mol_type = other DNA
cctcagtcatccttatcc

SEQ ID NO: 488 - DNA, 18 bp, synthetic construct
  misc_feature 1..18: note = Synthetic Construct
  source 1..18: mol_type = other DNA
ccctgaagtttcagttgt

SEQ ID NO: 489 - DNA, 228 bp, synthetic construct
  misc_feature 1..228: note = Synthetic Construct
  source 1..228: mol_type = other DNA
aatgaaatcttctgatttgtaagacatgctgccaagagattagttttacaccttcaggataaagactgctgagaaggtttaaggatgctattctgaaaagactgtggaggaagattaaggaaaccatctctgggataaggatgactgaggaaatttaaggatgccactctggttaaaagctgaaacaactgaaacttcagggaaaagagaaggcctggaatctgatcc

SEQ ID NO: 490 - DNA, 79 bp, synthetic construct
  misc_feature 1..79: note = Synthetic Construct
  source 1..79: mol_type = other DNA
gggtcaatgatgacaacccaatgtcatgaagaaatgtgatgacataaaatttatgctcaataggattacgctgagtccc

SEQ ID NO: 491 - DNA, 79 bp, synthetic construct
  misc_feature 1..79: note = Synthetic Construct
  source 1..79: mol_type = other DNA
gggtcaataatggcaatccaatgtcatgaagaaaggtgatgacataaaattcatgctcaataggattactctgaggccc

SEQ ID NO: 492 - DNA, 88 bp, synthetic construct
  misc_feature 1..88: note = Synthetic Construct
  source 1..88: mol_type = other DNA
gggttcacaaagtgcaatccctcgagccaatgtcatgaagaaaggtgatgacataaaattcatgctcaataggattatgctgaggccc

SEQ ID NO: 493 - DNA, 79 bp, synthetic construct
  misc_feature 1..79: note = Synthetic Construct
  source 1..79: mol_type = other DNA
ttgtcaatgataacaacccaaaatcatgaacagaggtgatgatataaaaatcatgctcaataggattacgctgaggcac

SEQ ID NO: 494 - DNA, 69 bp, synthetic construct
  misc_feature 1..69: note = Synthetic Construct
  source 1..69: mol_type = other DNA
tggccatcataggtcatgaagagtggtgatgacattaaaatcatgatcaataggattacactgaggccc

SEQ ID NO: 495 - DNA, 82 bp, synthetic construct
  misc_feature 1..82: note = Synthetic Construct
  source 1..82: mol_type = other DNA
gggtcaatgatgagaaccttatattgtcctgaagagcggtgatgacttaaaaatcatgctcaataggattacgctgaggccc

SEQ ID NO: 496 - DNA, 76 bp, synthetic construct
  misc_feature 1..76: note = Synthetic Construct
  source 1..76: mol_type = other DNA
gggtcaatgatgagatgttaccttgaagagaaatgatgacgtaaaaattaagttcagttggattacgctgaggccc

SEQ ID NO: 497 - DNA, 53 bp, synthetic construct
  misc_feature 1..53: note = Synthetic Construct
  source 1..53: mol_type = other DNA
ttatattgtcttcgacagggaagatgacataaaaattatgttcaataggatta

SEQ ID NO: 498 - DNA, 71 bp, synthetic construct
  misc_feature 1..71: note = Synthetic Construct
  source 1..71: mol_type = other DNA
tcaataatgaaatcttctgatttggtgagaaataatgccttaaaattacactcaataggattatgctgagg

SEQ ID NO: 499 - DNA, 33 bp, synthetic construct
  misc_feature 1..33: note = Synthetic Construct
  source 1..33: mol_type = other DNA
taaaaatcatgctcaatagaattaagctgaggc

SEQ ID NO: 500 - DNA, 20 bp, synthetic construct
  misc_feature 1..20: note = Synthetic Construct
  source 1..20: mol_type = other DNA
atatgtggaagccggaatct

SEQ ID NO: 501 - DNA, 21 bp, synthetic construct
  misc_feature 1..21: note = Synthetic Construct
  source 1..21: mol_type = other DNA
cccagaactccctaatcagaa

SEQ ID NO: 502 - DNA, 20 bp, synthetic construct
  misc_feature 1..20: note = Synthetic Construct
  source 1..20: mol_type = other DNA
atgacggtggctataccagg

SEQ ID NO: 503 - DNA, 20 bp, synthetic construct
  misc_feature 1..20: note = Synthetic Construct
  source 1..20: mol_type = other DNA
gtctccttcgagctgtttgc

SEQ ID NO: 504 - DNA, 21 bp, synthetic construct
  misc_feature 1..21: note = Synthetic Construct
  source 1..21: mol_type = other DNA
cctttctctccagtgctcaga

SEQ ID NO: 505 - DNA, 21 bp, synthetic construct
  misc_feature 1..21: note = Synthetic Construct
  source 1..21: mol_type = other DNA
cctgtgaactttcaaccagga

SEQ ID NO: 506 - DNA, 20 bp, synthetic construct
  misc_feature 1..20: note = Synthetic Construct
  source 1..20: mol_type = other DNA
ggatcagactccaggccttc

SEQ ID NO: 507 - RNA, 20 nt, synthetic construct
  misc_feature 1..20: note = Synthetic Construct
  source 1..20: mol_type = other RNA
cccaggtgtcctttaatgaa

SEQ ID NO: 508 - RNA, 20 nt, synthetic construct
  misc_feature 1..20: note = Synthetic Construct
  source 1..20: mol_type = other RNA
ccaggtgtcctttaatgaaa

SEQ ID NO: 509 - RNA, 20 nt, synthetic construct
  misc_feature 1..20: note = Synthetic Construct
  source 1..20: mol_type = other RNA
tgaaaatgctcttgacacca

SEQ ID NO: 510 - RNA, 20 nt, synthetic construct
  misc_feature 1..20: note = Synthetic Construct
  source 1..20: mol_type = other RNA
gaaaatgctcttgacaccaa

SEQ ID NO: 511 - RNA, 20 nt, synthetic construct
  misc_feature 1..20: note = Synthetic Construct
  source 1..20: mol_type = other RNA
aaatgctcttgacaccaatg

SEQ ID NO: 512 - RNA, 20 nt, synthetic construct
  misc_feature 1..20: note = Synthetic Construct
  source 1..20: mol_type = other RNA
agatcagtagcttcctttac

SEQ ID NO: 513 - RNA, 20 nt, synthetic construct
  misc_feature 1..20: note = Synthetic Construct
  source 1..20: mol_type = other RNA
tcagtagcttcctttaccga

SEQ ID NO: 514 - RNA, 20 nt, synthetic construct
  misc_feature 1..20: note = Synthetic Construct
  source 1..20: mol_type = other RNA
tctagaacattgagctatgg

SEQ ID NO: 515 - RNA, 20 nt, synthetic construct
  misc_feature 1..20: note = Synthetic Construct
  source 1..20: mol_type = other RNA
ctagaacattgagctatgga

SEQ ID NO: 516 - RNA, 20 nt, synthetic construct
  source 1..20: mol_type = other RNA
aacattgagctatggaagac

SEQ ID NO: 517 - RNA, 20 nt, synthetic construct
  misc_feature 1..20: note = Synthetic Construct
  source 1..20: mol_type = other RNA
acattgagctatggaagact

SEQ ID NO: 518 - RNA, 20 nt, synthetic construct
  misc_feature 1..20: note = Synthetic Construct
  source 1..20: mol_type = other RNA
ctatggaagactcccaccta

SEQ ID NO: 519 - RNA, 20 nt, synthetic construct
  misc_feature 1..20: note = Synthetic Construct
  source 1..20: mol_type = other RNA
tatggaagactcccacctaa

SEQ ID NO: 520 - RNA, 20 nt, synthetic construct
  misc_feature 1..20: note = Synthetic Construct
  source 1..20: mol_type = other RNA
caagtgctaccgcacaggca

SEQ ID NO: 521 - RNA, 20 nt, synthetic construct
  misc_feature 1..20: note = Synthetic Construct
  source 1..20: mol_type = other RNA
aagtgctaccgcacaggcat

SEQ ID NO: 522 - RNA, 20 nt, synthetic construct
  misc_feature 1..20: note = Synthetic Construct
  source 1..20: mol_type = other RNA
taccgcacaggcatgctgca

SEQ ID NO: 523 - RNA, 20 nt, synthetic construct
  misc_feature 1..20: note = Synthetic Construct
  source 1..20: mol_type = other RNA
caggcatgctgcagtgaatt

SEQ ID NO: 524 - RNA, 20 nt, synthetic construct
  misc_feature 1..20: note = Synthetic Construct
  source 1..20: mol_type = other RNA
aggcatgctgcagtgaattt

SEQ ID NO: 525 - RNA, 20 nt, synthetic construct
  misc_feature 1..20: note = Synthetic Construct
  source 1..20: mol_type = other RNA
accgttgtttaaggatgcta

SEQ ID NO: 526 - RNA, 20 nt, synthetic construct
  misc_feature 1..20: note = Synthetic Construct
  source 1..20: mol_type = other RNA
ccgttgtttaaggatgctat

SEQ ID NO: 527 - RNA, 20 nt, synthetic construct
  misc_feature 1..20: note = Synthetic Construct
  source 1..20: mol_type = other RNA
ctgtggaggaagaaaaccct

SEQ ID NO: 528 - RNA, 20 nt, synthetic construct
  misc_feature 1..20: note = Synthetic Construct
  source 1..20: mol_type = other RNA
aagaaaaccctttaccctgt

SEQ ID NO: 529 - RNA, 20 nt, synthetic construct
  misc_feature 1..20: note = Synthetic Construct
  source 1..20: mol_type = other RNA
agaaaaccctttaccctgtt

SEQ ID NO: 530 - RNA, 20 nt, synthetic construct
  misc_feature 1..20: note = Synthetic Construct
  source 1..20: mol_type = other RNA
ctcaactgcctggcactgaa

SEQ ID NO: 531 - RNA, 20 nt, synthetic construct
  misc_feature 1..20: note = Synthetic Construct
  source 1..20: mol_type = other RNA
aactgcctggcactgaaaat

SEQ ID NO: 532 - RNA, 20 nt, synthetic construct
  misc_feature 1..20: note = Synthetic Construct
  source 1..20: mol_type = other RNA
actgcctggcactgaaaatg

SEQ ID NO: 533 - RNA, 20 nt, synthetic construct
  misc_feature 1..20: note = Synthetic Construct
  source 1..20: mol_type = other RNA
gtgtttaaggaaaccatctc

SEQ ID NO: 534 - RNA, 20 nt, synthetic construct
  misc_feature 1..20: note = Synthetic Construct
  source 1..20: mol_type = other RNA
gtttaaggaaaccatctctg

SEQ ID NO: 535 - RNA, 20 nt, synthetic construct
  misc_feature 1..20: note = Synthetic Construct
  source 1..20: mol_type = other RNA
aggaaaccatctctgataag

SEQ ID NO: 536 - RNA, 20 nt, synthetic construct
  misc_feature 1..20: note = Synthetic Construct
  source 1..20: mol_type = other RNA
tctttggcttgttgacacca

SEQ ID NO: 537 - RNA, 20 nt, synthetic construct
  misc_feature 1..20: note = Synthetic Construct
  source 1..20: mol_type = other RNA
ctttggcttgttgacaccag

SEQ ID NO: 538 - DNA, 20 bp, synthetic construct
  misc_feature 1..20: note = Synthetic Construct
  source 1..20: mol_type = other DNA
ttcattaaaggacacctggg

SEQ ID NO: 539 - DNA, 20 bp, synthetic construct
  misc_feature 1..20: note = Synthetic Construct
  source 1..20: mol_type = other DNA
tttcattaaaggacacctgg

SEQ ID NO: 540 - DNA, 20 bp, synthetic construct
  misc_feature 1..20: note = Synthetic Construct
  source 1..20: mol_type = other DNA
tggtgtcaagagcattttca

SEQ ID NO: 541 - DNA, 20 bp, synthetic construct
  misc_feature 1..20: note = Synthetic Construct
  source 1..20: mol_type = other DNA
ttggtgtcaagagcattttc

SEQ ID NO: 542 - DNA, 20 bp, synthetic construct
  misc_feature 1..20: note = Synthetic Construct
  source 1..20: mol_type = other DNA
cattggtgtcaagagcattt

SEQ ID NO: 543 - DNA, 20 bp, synthetic construct
  misc_feature 1..20: note = Synthetic Construct
  source 1..20: mol_type = other DNA
gtaaaggaagctactgatct

SEQ ID NO: 544 - DNA, 20 bp, synthetic construct
  misc_feature 1..20: note = Synthetic Construct
  source 1..20: mol_type = other DNA
tcggtaaaggaagctactga

SEQ ID NO: 545 - DNA, 20 bp, synthetic construct
  misc_feature 1..20: note = Synthetic Construct
  source 1..20: mol_type = other DNA
ccatagctcaatgttctaga

SEQ ID NO: 546 - DNA, 20 bp, synthetic construct
  misc_feature 1..20: note = Synthetic Construct
  source 1..20: mol_type = other DNA
tccatagctcaatgttctag

SEQ ID NO: 547 - DNA, 20 bp, synthetic construct
  misc_feature 1..20: note = Synthetic Construct
  source 1..20: mol_type = other DNA
gtcttccatagctcaatgtt

SEQ ID NO: 548 - DNA, 20 bp, synthetic construct
  misc_feature 1..20: note = Synthetic Construct
  source 1..20: mol_type = other DNA
agtcttccatagctcaatgt

SEQ ID NO: 549 - DNA, 20 bp, synthetic construct
  misc_feature 1..20: note = Synthetic Construct
  source 1..20: mol_type = other DNA
taggtgggagtcttccatag

SEQ ID NO: 550 - DNA, 20 bp, synthetic construct
  misc_feature 1..20: note = Synthetic Construct
  source 1..20: mol_type = other DNA
ttaggtgggagtcttccata

SEQ ID NO: 551 - DNA, 20 bp, synthetic construct
  misc_feature 1..20: note = Synthetic Construct
  source 1..20: mol_type = other DNA
tgcctgtgcggtagcacttg

SEQ ID NO: 552 - DNA, 20 bp, synthetic construct
  misc_feature 1..20: note = Synthetic Construct
  source 1..20: mol_type = other DNA
atgcctgtgcggtagcactt

SEQ ID NO: 553 - DNA, 20 bp, synthetic construct
  misc_feature 1..20: note = Synthetic Construct
  source 1..20: mol_type = other DNA
tgcagcatgcctgtgcggta

SEQ ID NO: 554 - DNA, 20 bp, synthetic construct
  misc_feature 1..20: note = Synthetic Construct
  source 1..20: mol_type = other DNA
aattcactgcagcatgcctg

SEQ ID NO: 555 - DNA, 20 bp, synthetic construct
  misc_feature 1..20: note = Synthetic Construct
  source 1..20: mol_type = other DNA
aaattcactgcagcatgcct

SEQ ID NO: 556 - DNA, 20 bp, synthetic construct
  misc_feature 1..20: note = Synthetic Construct
  source 1..20: mol_type = other DNA
tagcatccttaaacaacggt

SEQ ID NO: 557 - DNA, 20 bp, synthetic construct
  misc_feature 1..20: note = Synthetic Construct
  source 1..20: mol_type = other DNA
atagcatccttaaacaacgg

SEQ ID NO: 558 - DNA, 20 bp, synthetic construct
  misc_feature 1..20: note = Synthetic Construct
  source 1..20: mol_type = other DNA
agggttttcttcctccacag

SEQ ID NO: 559 - DNA, 20 bp, synthetic construct
  misc_feature 1..20: note = Synthetic Construct
  source 1..20: mol_type = other DNA
acagggtaaagggttttctt

SEQ ID NO: 560 - DNA, 20 bp, synthetic construct
  misc_feature 1..20: note = Synthetic Construct
  source 1..20: mol_type = other DNA
aacagggtaaagggttttct

SEQ ID NO: 561 - DNA, 20 bp, synthetic construct
  misc_feature 1..20: note = Synthetic Construct
  source 1..20: mol_type = other DNA
ttcagtgccaggcagttgag

SEQ ID NO: 562 - DNA, 20 bp, synthetic construct
  misc_feature 1..20: note = Synthetic Construct
  source 1..20: mol_type = other DNA
attttcagtgccaggcagtt

SEQ ID NO: 563 - DNA, 20 bp, synthetic construct
  misc_feature 1..20: note = Synthetic Construct
  source 1..20: mol_type = other DNA
cattttcagtgccaggcagt

SEQ ID NO: 564 - DNA, 20 bp, synthetic construct
  misc_feature 1..20: note = Synthetic Construct
  source 1..20: mol_type = other DNA
gagatggtttccttaaacac

SEQ ID NO: 565 - DNA, 20 bp, synthetic construct
  misc_feature 1..20: note = Synthetic Construct
  source 1..20: mol_type = other DNA
cagagatggtttccttaaac

SEQ ID NO: 566 - DNA, 20 bp, synthetic construct
  misc_feature 1..20: note = Synthetic Construct
  source 1..20: mol_type = other DNA
cttatcagagatggtttcct

SEQ ID NO: 567 - DNA, 20 bp, synthetic construct
  misc_feature 1..20: note = Synthetic Construct
  source 1..20: mol_type = other DNA
tggtgtcaacaagccaaaga

SEQ ID NO: 568 - DNA, 20 bp, synthetic construct
  misc_feature 1..20: note = Synthetic Construct
  source 1..20: mol_type = other DNA
ctggtgtcaacaagccaaag

SEQ ID NO: 569 - DNA, 58 bp, synthetic construct
  misc_feature 1..58: note = Synthetic Construct
  source 1..58: mol_type = other DNA
ggtgccattctattataaataacctgacccattatttataatagaatggcaccttttt

SEQ ID NO: 570 - DNA, 58 bp, synthetic construct
  misc_feature 1..58: note = Synthetic Construct
  source 1..58: mol_type = other DNA
gctttcatcaataatgaaataacctgacccattatttcattattgatgaaagcttttt

SEQ ID NO: 571 - DNA, 58 bp, synthetic construct
  misc_feature 1..58: note = Synthetic Construct
  source 1..58: mol_type = other DNA
ggtctttcatcaataatgataacctgacccattatcattattgatgaaagaccttttt

SEQ ID NO: 572 - DNA, 58 bp, synthetic construct
  misc_feature 1..58: note = Synthetic Construct
  source 1..58: mol_type = other DNA
gaaatcttctgatttgtaataacctgacccattattacaaatcagaagatttcttttt

SEQ ID NO: 573 - DNA, 58 bp, synthetic construct
  misc_feature 1..58: note = Synthetic Construct
  source 1..58: mol_type = other DNA
gcacctaagggaattagtataacctgacccattatactaattcccttaggtgcttttt

SEQ ID NO: 574 - DNA, 58 bp, synthetic construct
  misc_feature 1..58: note = Synthetic Construct
  source 1..58: mol_type = other DNA
gtttcaaccaggatttaaataacctgacccattatttaaatcctggttgaaacttttt

SEQ ID NO: 575 - DNA, 58 bp, synthetic construct
  misc_feature 1..58: note = Synthetic Construct
  source 1..58: mol_type = other DNA
gctttcaaccaggatttaataacctgacccattattaaatcctggttgaaagcttttt

SEQ ID NO: 576 - DNA, 58 bp, synthetic construct
  misc_feature 1..58: note = Synthetic Construct
  source 1..58: mol_type = other DNA
ggagatgtgccattctatataacctgacccattatatagaatggcacatctccttttt

SEQ ID NO: 577 - DNA, 58 bp, synthetic construct
  misc_feature 1..58: note = Synthetic Construct
  source 1..58: mol_type = other DNA
gtctttcatcaataatgaataacctgacccattattcattattgatgaaagacttttt

SEQ ID NO: 578 - DNA, 58 bp, synthetic construct
  misc_feature 1..58: note = Synthetic Construct
  source 1..58: mol_type = other DNA
gatcaataatgaaatcttataacctgacccattataagatttcattattgatcttttt

SEQ ID NO: 579 - DNA, 58 bp, synthetic construct
  misc_feature 1..58: note = Synthetic Construct
  source 1..58: mol_type = other DNA
gtgtctttcatcaataatataacctgacccattatattattgatgaaagacacttttt

SEQ ID NO: 580 - DNA, 58 bp, synthetic construct
  misc_feature 1..58: note = Synthetic Construct
  source 1..58: mol_type = other DNA
gcaataatgaaatcttctataacctgacccattatagaagatttcattattgcttttt

SEQ ID NO: 581 - DNA, 58 bp, synthetic construct
  misc_feature 1..58: note = Synthetic Construct
  source 1..58: mol_type = other DNA
gcatgctgcagtgaatttataacctgacccattataaattcactgcagcatgcttttt

SEQ ID NO: 582 - DNA, 58 bp, synthetic construct
  misc_feature 1..58: note = Synthetic Construct
  source 1..58: mol_type = other DNA
ggaaatcttctgatttgtataacctgacccattatacaaatcagaagatttccttttt

SEQ ID NO: 583 - DNA, 58 bp, synthetic construct
  misc_feature 1..58: note = Synthetic Construct
  source 1..58: mol_type = other DNA
ggtatattctatctagaaataacctgacccattatttctagatagaatataccttttt

SEQ ID NO: 584 - DNA, 58 bp, synthetic construct
  misc_feature 1..58: note = Synthetic Construct
  source 1..58: mol_type = other DNA
gtgctgcagtgaatttaaataacctgacccattatttaaattcactgcagcacttttt

SEQ ID NO: 585 - DNA, 58 bp, synthetic construct
  misc_feature 1..58: note = Synthetic Construct
  source 1..58: mol_type = other DNA
gtgtgccattctattataataacctgacccattattataatagaatggcacacttttt

SEQ ID NO: 586 - DNA, 58 bp, synthetic construct
  misc_feature 1..58: note = Synthetic Construct
  source 1..58: mol_type = other DNA
gttaccatcagtgtttaaataacctgacccattatttaaacactgatggtaacttttt

SEQ ID NO: 587 - DNA, 58 bp, synthetic construct
  misc_feature 1..58: note = Synthetic Construct
  source 1..58: mol_type = other DNA
gcctgcaaccgttgtttaataacctgacccattattaaacaacggttgcaggcttttt

SEQ ID NO: 588 - DNA, 58 bp, synthetic construct
  misc_feature 1..58: note = Synthetic Construct
  source 1..58: mol_type = other DNA
gtatgtctttcatcaataataacctgacccattattattgatgaaagacatacttttt

SEQ ID NO: 589 - DNA, 20 bp, synthetic construct
  misc_feature 1..20: note = Synthetic Construct
  source 1..20: mol_type = other DNA
acactgatggtaaagtggac

SEQ ID NO: 590 - DNA, 20 bp, synthetic construct
  misc_feature 1..20: note = Synthetic Construct
  source 1..20: mol_type = other DNA
tagaatatacacgtcggtaa

SEQ ID NO: 591 - DNA, 20 bp, synthetic construct
  misc_feature 1..20: note = Synthetic Construct
  source 1..20: mol_type = other DNA
tcaactgtcccagtcacaac

SEQ ID NO: 592 - DNA, 20 bp, synthetic construct
  misc_feature 1..20: note = Synthetic Construct
  source 1..20: mol_type = other DNA
tctagatagaatatacacgt

SEQ ID NO: 593 - DNA, 20 bp, synthetic construct
  misc_feature 1..20: note = Synthetic Construct
  source 1..20: mol_type = other DNA
tctagatagaatatacacgt

SEQ ID NO: 594 - DNA, 20 bp, synthetic construct
  misc_feature 1..20: note = Synthetic Construct
  source 1..20: mol_type = other DNA
ctccccatgcacacttgaga

SEQ ID NO: 595 - DNA, 20 bp, synthetic construct
  misc_feature 1..20: note = Synthetic Construct
  source 1..20: mol_type = other DNA
catccttaaacaacggttgc

SEQ ID NO: 596 - DNA, 20 bp, synthetic construct
  misc_feature 1..20: note = Synthetic Construct
  source 1..20: mol_type = other DNA
ggtgtaaaactaattccctt

SEQ ID NO: 597 - DNA, 20 bp, synthetic construct
  misc_feature 1..20: note = Synthetic Construct
  source 1..20: mol_type = other DNA
aacaacggttgcagggacag

SEQ ID NO: 598 - DNA, 20 bp, synthetic construct
  misc_feature 1..20: note = Synthetic Construct
  source 1..20: mol_type = other DNA
tatggaagactcccacctaa

SEQ ID NO: 599 - DNA, 20 bp, synthetic construct
  misc_feature 1..20: note = Synthetic Construct
  source 1..20: mol_type = other DNA
ctatggaagactcccaccta

SEQ ID NO: 600 - DNA, 20 bp, synthetic construct
  misc_feature 1..20: note = Synthetic Construct
  source 1..20: mol_type = other DNA
aagccttctcaagtgtgcat

SEQ ID NO: 601 - DNA, 20 bp, synthetic construct
  misc_feature 1..20: note = Synthetic Construct
  source 1..20: mol_type = other DNA
ctatctagaacattgagcta

SEQ ID NO: 602 - DNA, 20 bp, synthetic construct
  misc_feature 1..20: note = Synthetic Construct
  source 1..20: mol_type = other DNA
accctctggtgttgtcacag

SEQ ID NO: 603 - DNA, 20 bp, synthetic construct
  misc_feature 1..20: note = Synthetic Construct
  source 1..20: mol_type = other DNA
aaccctttaccctgttgttc

SEQ ID NO: 604 - DNA, 79 bp, synthetic construct
  source 1..79: mol_type = other DNA
gggtcaataatgncaanccaatgtcatgaagaaaggtgatgacataaaattcatgctcaataggattacgctgaggccc

SEQ ID NO: 605 - DNA, 78 bp, synthetic construct
  source 1..78: mol_type = other DNA
ggtcaatgatgagacttatnttgtcttgaagagagatgatgacntaaaaattatgctcaataggattacgctgaggcc